(19) United States
(12) Patent Application Publication
(10) Pub. No.: US 2015/0329841 A1
(43) Pub. Date: Nov. 19, 2015

(54) CELLULOSE-DEGRADING ENZYME COMPOSITION COMPRISING GH16

(71) Applicant: Iogen Energy Corporation, Ottawa (CA)

(72) Inventors: Christopher M. D. Hill, Nepean (CA); John J. Tomashek, Ottawa (CA); James A. Lavigne, Nepean (CA); Sandra Mortimer, Orleans (CA); Reginald Storms, Beaconsfield (CA); Adrian Tsang, Montreal (CA); Greg Butler, Montreal (CA); Justin Powlowski, Montreal (CA)

(21) Appl. No.: 14/381,034

(22) PCT Filed: May 31, 2013
(86) PCT No.: PCT/CA2013/050421

§ 371 Date: Aug. 26, 2014

Related U.S. Application Data
(60) Provisional application No. 61/653,649, filed on May 31, 2012.

(51) Int. Cl. C12N 9/42; C12P 19/02; C12P 19/14

(52) U.S. Cl. CPC C12N 9/2437; C12N 9/2434; C12P 19/14; C12P 19/02; C12P 2203/00; C12Y 302/01004; C12Y 302/01091

(57) ABSTRACT

The present invention relates to a cellulose-degrading enzyme composition comprising one or more cellobiohydrolase and/or endoglucanase enzyme, and an effective amount of an isolated GH16 polypeptide, where the presence of the isolated GH16 polypeptide in the enzyme composition increases the rate or extent of degradation of a cellulosic substrate compared to an equivalent dosage of a corresponding cellulose-degrading enzyme composition lacking the at least one isolated GH16 polypeptide. The present invention also relates to a method for producing fermentable sugars from a cellulosic substrate using the above cellulose-degrading enzyme composition and to genetically modified microbes for produced the above cellulose-degrading enzyme composition.

### FIELD OF THE INVENTION

The present invention provides a cellulose-degrading enzyme composition, a method for treating a cellulose substrate with the cellulose-degrading enzyme composition to produce fermentable sugars, and genetically modified microbes for producing the cellulose-degrading enzyme composition.

### BACKGROUND OF THE INVENTION

Lignocellulosic feedstocks are a promising alternative or complement to corn or wheat starch, sugar cane and sugar beets for the production of fuel ethanol. Lignocellulosic feedstocks are widely available, inexpensive and several studies have concluded that cellulosic ethanol generates close to zero greenhouse gas emissions.

However, lignocellulosic feedstocks are not easily broken down into their composite sugar molecules. Recalcitrance of lignocellulose can be partially overcome by physical and/or chemical pretreatment. An example of a chemical pretreatment is steam explosion in the presence of dilute sulfuric acid (U.S. Pat. No. 4,461,648). This process removes most of the hemicellulose, but there is little conversion of the cellulose to glucose. The pretreated material may then be hydrolyzed by cellulase enzymes.

The term cellulase (or cellulase enzymes) broadly refers to enzymes that catalyze the hydrolysis of the β-1,4-glucosidic bonds joining individual glucose units in the cellulose polymer. The catalytic mechanism involves the synergistic actions of endoglucanases (E.C. 3.2.1.4), cellobiohydrolases (E.C. 3.2.1.91) and beta-glucosidase (E.C. 3.2.1.21). Endoglucanases hydrolyze accessible glucosidic bonds in the middle of the cellulose chain, while cellobiohydrolases release cellobiose from these chain ends processively. Beta-glucosidases hydrolyze cellobiose to glucose and, in doing so, minimize product inhibition of the cellobiohydrolases. Collectively, the enzymes operate as a composition that can hydrolyze a cellulose substrate.

Cellulase enzymes may be obtained from filamentous fungi, including species of Trichoderma, Hypocrea, Aspergillus, Chaetomium, Chrysosporium, Coprinus, Corynascus, Fomitopsis, Fusarium, Humicola, Magnaporthe, Melanocarpus, Myceliophthora, Neurospora, Phanerochaete, Podospora, Rhizomucor, Sporotrichum, Talaromyces, Thermoascus, Thermomyces and Thielavia.

For example, the industrially relevant filamentous fungus Trichoderma reesei, the anamorph of Hypocrea jecorina, secretes two cellobiohydrolase (CBH) enzymes, CBH1 (Cel7A) and CBH2 (Cel6A), which release cellobiose from reducing and non-reducing ends of the cellulose chain, respectively, several beta-glucosidase enzymes (including beta-glucosidase I or Cel3A), and several endoglucanase (EG) enzymes. EG1 (Cel7B) and EG2 (Cel5A) are two major endoglucanases involved in the hydrolysis of crystalline cellulose. CBH1 (Cel7A), CBH2 (Cel6A), EG1 (Cel7B) and EG2 (Cel5A) comprise two functional domains, namely a catalytic domain and a carbohydrate binding module (CBM). Of the remaining endoglucanases, EG3 (Cell2A) lacks a carbohydrate binding module and therefore binds crystalline cellulose poorly (Karlsson et al., 2002a, Journal of Biotechnology, 99:63-78). EG5 (Cel45A) and EG6 (Cel74A) are reported to be a glucomannanase (Karlsson et al., 2002a) and a xyloglucanase (Desmet et al., 2006, FEBS Journal, 274:356-363, respectively).

Myceliophthora thermophila, the anamorph of Thielavia heterothallica, produces a more complex cellulase enzyme system including at least four cellobiohydrolases (CBH1a, CBH1b, CBH2a, and CBH2b), several endoglucanases (including EG1a, EG1b, EG2), several beta-glucosidases, and over twenty proteins belonging to Glycoside Hydrolase (GH) Family 61 (Visser, H., et al., 2011, Industrial Biotech. 7(3): 214-223).

The EG4 (Cel61A or GH61A) protein from T. reesei was initially reported to exhibit some activity on carboxymethyl cellulose, hydroxyethyl cellulose and beta-glucan (Karlsson et al., 2002b, European Journal of Biochemistry, 268:6498-6507). More recently, Trichoderma reesei Cel61B (U.S. Pat. No. 7,608,869), as well as GH61 proteins from a variety of organisms, including Myceliophthora thermophila (U.S. Publication Nos. 2010/0306881A1, 2010/0304434A1, 2010/0299789A1, and 2010/0299788A1), Thielavia terrestris (U.S. Pat. Nos. 7,741,466, 7,361,495 and 7,273,738; U.S. Publication Nos. 2010/0143967A1, 2010/0129860A1, 2010/0197556A1, 2011/0296558A1, and 2012/0011619A1; and WO2011/035072A2), Thermoascus aurantiacus (WO2011/0415504A1, WO2011/039319A1, and U.S. Pat. No. 7,868,227), and species of Penicillium (WO2011/005867A1 and WO2011/041397A1) have been shown to enhance the cellulose degradation by cellulase enzymes. Recent studies suggest that GH61 proteins are polysaccharide mono-oxygenases that are dependent on copper or other divalent metal cations (Beeson, et al., 2012, J. Am. Chem. Soc. 134: 890-892; Beeson, et al., 2011, ACS Chem. Biol. 6: 1399-1406; and WO2012/019151 A1).

The enzymatic hydrolysis of pretreated lignocellulosic feedstocks is an inefficient step in the production of cellulosic ethanol and its cost constitutes one of the major barriers to commercial viability Improving the enzymatic activity of cellulases or increasing cellulase production efficiency has been widely regarded as an opportunity for significant cost savings.

Numerous approaches have been taken to improve the activity of cellulase for ethanol production. The amount of beta-glucosidase activity secreted by Trichoderma has been increased in order to minimize cellobiose accumulation and product inhibition (U.S. Pat. No. 6,015,703). Mutagenesis strategies have been used to improve the thermostability of CBH1 (WO2005/0277172) and CBH2 (US 2006/0205042) Amino acid consensus and mutagenesis strategies have been employed to improve the activity of CBH1 (WO2004/0197890) and CBH2 (WO2006/0053514). A fusion protein consisting of the Cel7A catalytic domain from T. reesei and the EG1 catalytic domain from Acidothermus cellulolyticus has been constructed (WO2006/00057672). Additionally, novel combinations of CBMs and catalytic domains from cellulases and hemicellulases originating from Myceliophthora, Humicola and Fusarium have been generated by domain shuffling in an attempt to generate enzymes with novel enzyme specificities and activities (U.S. Pat. No. 5,763,254).

These approaches focused on individual cellulase components, in particular those exhibiting substantial activity on laboratory substrates such as filter paper, carboxymethyl cellulose (CMC), hydroxyethyl cellulose (HEC), and beta-glucan. While altering the properties of an individual protein, these approaches have not increased substantially the activity of cellulose-degrading enzyme compositions. Thus, neither the amount of enzyme used for producing fermentable sugars from lignocellulose nor the cost of the enzyme have been reduced substantially by approaches directed to single components within the cellulose-degrading composition.

Some studies have tested hemicellulases in conjunction with a cellulase preparation for improved activity on lignocellulosic substrates (Berlin et al., 2007, Biotechnology and Bioengineering, 97(2): 287-296). Such enzyme mixtures are useful for lignocellulosic substrates in which a significant fraction is hemicellulose, such as substrates prepared by alkaline pre-treatment methods. However, for lignocellulosic substrates with low hemicellulose content, such as those produced by acid pretreatment processes, hemicellulase-enriched enzyme mixtures may not be more effective on these substrates than cellulase mixtures.

Some Trichoderma cellulase components have negligible hydrolytic activity on laboratory cellulose-mimetic substrates, but are induced by cellulose. Cip1 and Cip2 are induced by cellulose and sophorose, implying that they have roles in the breakdown of cellulosic biomass, yet their activities are unknown (Foreman et al., 2003, Journal of Biological Chemistry, 278(34) 31988-31997). Swollenin (Swo1), a novel fungal protein containing an expansin domain and a CBM, has been shown to disrupt cotton fibers (Saloheimo et al., 2002, European Journal of Biochemistry, 269:4202-4211), presumably by breaking hydrogen bonds in the cellulose structure.

In spite of much research effort, there remains a need for an improved cellulose-degrading enzyme composition for the hydrolysis of cellulose in a pretreated lignocellulosic feedstock. The absence of such a composition represents a large hurdle in the commercialization of cellulose conversion to fermentable sugars including glucose for the production of ethanol and other products.

### SUMMARY OF THE INVENTION

The present invention provides a cellulose-degrading enzyme composition. The present invention also provides a method for treating a cellulose substrate with the cellulose-degrading enzyme composition to produce fermentable sugars and genetically modified microbes for producing the cellulose-degrading enzyme composition.

In a first aspect of the present invention, there is provided a cellulose-degrading enzyme composition which comprises one or more cellobiohydrolase or endoglucanase enzymes, and an effective amount of an isolated GH16 polypeptide, where the presence of the isolated GH16 polypeptide in the enzyme composition increases the rate or extent of degradation of a cellulose substrate compared to an equivalent dosage of a cellulose-degrading enzyme composition comprising the same one or more cellobiohydrolase or endoglucanase enzyme but lacking the isolated GH16 polypeptide.

In another aspect of the present invention, there is provided a cellulose-degrading enzyme composition which comprises one or more cellobiohydrolase enzymes, one or more endoglucanase enzymes, and an effective amount of a isolated GH16 polypeptide, where the presence of the isolated GH16 polypeptide in the enzyme composition increases the rate or extent of degradation of a cellulose substrate compared to an equivalent dosage of a cellulose-degrading enzyme composition comprising the same one or more cellobiohydrolase or endoglucanase enzyme but lacking the isolated GH16 polypeptide.

In some embodiments, the source of the isolated GH16 polypeptide is one or more of Gloeophyllum trabeum, Geomyces pannorum, Coprinus cinereus, Leucosporidium scottii, Phanerochaete chrysosporium, Schizophylum commune, Laccaria bicolor, Serpula lacrymans, Piriformospora indica, Postia placenta, Aspergillus fumigatus, Aspergillus nidulans, Rhodotorula glutinis, Lentiula edodes, Cryptococcus neoformans, and taxonomic equivalents thereof. For example, the isolated GH16 polypeptide may be from Gloeophyllum trabeum (e.g., the Gtra GH16 polypeptide of SEQ ID NO: 3), from Geomyces pannorum (e.g., the Gpan GH16 polypeptide of SEQ ID NO: 7), from Coprinus cinereus (e.g., the Ccin GH16 polypeptide of SEQ ID NO: 5), from Leucosporidium scottii (e.g., the Lsco GH16 polypeptide of SEQ ID NO: 4), or from Phanerochaete chrysosporium (e.g., the Pchr GH16 polypeptide of SEQ ID NO: 6).

In some embodiments, the isolated GH16 polypeptide comprises an amino acid sequence exhibiting from about 35% to 100% identity to SEQ ID NO: 3 or SEQ ID NO: 4, from about 50% to 100% identity to SEQ ID NO: 5, from about 55% to 100% identity to SEQ NO: 6, or from about 40% to 100% identity to SEQ ID NO: 7. In other embodiments, the isolated GH16 polypeptide comprises the amino acid sequence of SEQ ID NO: 3, SEQ ID NO: 4, SEQ ID NO: 5, SEQ ID NO: 6, or SEQ ID NO: 7.

In some embodiments, the one or more cellobiohydrolase enzyme is a member of Glycoside Hydrolase (GH) Family 6 or 7 and the one or more endoglucanase enzyme is a member of Glycoside Hydrolase (GH) Family 5 or 7. In other embodiments, the cellobiohydrolase enzyme(s) and endoglucanase enzyme(s) are wild-type or variant enzymes of a fungal cell from the genus Trichoderma or Myceliophthora. For example, the cellobiohydrolase enzyme(s) and endoglucanase enzyme(s) are wild-type or variant enzymes of Trichoderma reesei or Myceliophthora thermophila.

In still other embodiments, the cellobiohydrolase of GH Family 7 comprises an amino acid sequence exhibiting from about 60% to 100% identity to amino acids 1-436 of SEQ ID NO: 9 or to amino acids 1 to 438 of SEQ ID NO: 20, the cellobiohydrolase of GH Family 6 comprises an amino acid sequence exhibiting from about 45% to 100% identity to amino acids 83-447 of SEQ ID NO: 10 or to amino acids 118-432 of SEQ ID NO: 23, the endoglucanase enzymes of GH Family 5 comprises an amino acid sequence exhibiting from about 40% to 100% identity to amino acids 202 to 222 of SEQ ID NO: 11 or from about 65% to 100% identity to amino acids 77 to 297 of SEQ ID NO: 22, and the endoglucanase of GH Family 7 comprises an amino acid sequence exhibiting from about 48% to 100% identity to amino acids 1 to 374 of SEQ ID NO: 16 or from about 65% to 100% identity to amino acids 30-390 of SEQ ID NO: 24.

In some embodiments, the cellulose-degrading enzyme composition further comprises a beta-glucosidase enzyme. In other embodiments, the cellulose-degrading enzyme composition further comprises a GH61 polypeptide. For example, the GH61 polypeptide may comprise an amino acid sequence exhibiting from about 50% to 100% identity to SEQ ID NO: 15, from about 55% to 100% identity to SEQ ID NO: 19, from about 65% to 100% identity to SEQ ID NO: 17, or from about 50% to 100% identity to SEQ ID NO: 18.

In other embodiments, the cellulose-degrading enzyme composition further comprises one or more hemicellulase (such as a xylanase, beta-mannanase, beta-xylosidase, beta-mannosidase, or alpha-L-arabinofuranosidase), one or more cellulase-enhancing protein (such as swollenin, CIP1, CIP2, or expansin), one or more lignin-degrading enzymes (such as laccase, lignin peroxidase, manganese peroxidase, or cellobiose dehydrogenase), or one or more esterases (such as acetyl xylan esterase or ferulic acid esterase).

According to a second aspect of the invention, there is provided a method for producing fermentable sugars comprising treating a cellulose substrate with the cellulose-degrading enzyme composition as defined above. In some embodiments, the cellulose substrate is a pretreated lignocellulose feedstock which may be, for example, corn stover, wheat straw, barley straw, rice straw, oat straw, canola straw, soybean stover, corn fiber, sugar beet pulp, pulp mill fines and rejects, sugar cane bagasse, sugar cane leaves and tops, hardwood, softwood, sawdust, switch grass, miscanthus, cord grass, and reed canary grass.

In a third aspect of the invention, there is provided a genetically modified microbe for producing a cellulose-degrading composition comprising, at least one polynucleotide encoding a cellobiohydrolase enzyme or an endoglucanase enzyme, and an isolated polynucleotide encoding an isolated GH16 polypeptide exhibiting from about 35% to 100% identity to SEQ ID NO: 3 or SEQ ID NO: 4, from about 50% to 100% identity to SEQ ID NO: 5, from about 55% to 100% identity to SEQ NO: 6, or from about 40% to 100% identity to SEQ ID NO: 7. In one embodiment, the isolated polynucleotide encodes an isolated GH16 polypeptide comprising the amino acid sequence of SEQ ID NO: 3, SEQ ID NO: 4, SEQ ID NO: 5, SEQ ID NO: 6, or SEQ ID NO: 7.

### BRIEF DESCRIPTION OF THE DRAWINGS

FIG. 1 is a phylogenetic tree showing the relationship of GH16 polypeptides from a number of fungal species.

FIG. 2 shows an amino acid sequence alignment of the GH16 polypeptides used to produce the phylogenetic tree of FIG. 1.

FIG. 3 is a map of vector pTr-Pc/xCcinGH16-Tcel6A-ble-TV used for the expression and secretion of the Coprinus cinereus GH16 polypeptide from genetically modified T. reesei strains.

FIG. 4 is a map of vector pTr-Pc/xGpanGH16-Tcel6A-ble-TV used for the expression and secretion of the Geomyces pannorum GH16 polypeptide from genetically modified T. reesei strains.

FIG. 5 is a map of vector pTr-Pc/xGtraGH16-Tcel6A-ble-TV used for the expression and secretion of the Gloeophyllum trabeum GH16 polypeptide from genetically modified T. reesei strains.

FIG. 6 is a map of vector pTr-Pc/xLscoGH16-Tcel6A-ble-TV used for the expression and secretion of the Leucosporidium scottii GH16 polypeptide from genetically modified T. reesei strains.

FIG. 7 is a map of vector pTr-Pc/xPchrGH16-Tcel6A-ble-TV used for the expression and secretion of the Phanerochaete chrysosporium GH16 polypeptide from genetically modified T. reesei strains.

FIG. 8 shows the relative activities of cellulose-degrading enzyme compositions comprising isolated GH16 polypeptides (Lsco GH16, Pchr GH16, Ccin GH16, Gpan GH16, or Gtra GH16) relative to an otherwise equivalent cellulose-degrading enzyme composition lacking an isolated GH16 polypeptide (control).

FIG. 9 is a map of vector ANIp5 used for the expression and secretion of the isolated GH16 polypeptides from genetically modified A. niger strains.

### DETAILED DESCRIPTION OF THE INVENTION

The present invention provides a cellulose-degrading enzyme composition. The present invention also provides a method for treating a cellulose substrate with the cellulose-degrading enzyme composition to produce fermentable sugars and genetically modified microbes for producing the cellulose-degrading enzyme composition.

The following description is of embodiments by way of example only and without limitation to the combination of features necessary for carrying the invention into effect. The headings provided are not meant to be limiting of the various embodiments of the invention. Terms such as “comprises,” “comprising,” “comprise,” “includes,” “including,” and “include” are not meant to be limiting. In addition, the use of the singular includes the plural, and “or” means “and/or” unless otherwise stated. Unless otherwise defined herein, all technical and scientific terms used herein have the same meaning as commonly understood by one of ordinary skill in the art.

### Cellulose-Degrading Enzyme Composition

As used herein, a cellulose-degrading enzyme composition is an enzyme mixture comprising at least one or more cellobiohydrolase (CBH) enzymes or endoglucanase (EG) enzymes, and an effective amount of an isolated GH16 polypeptide.

An “effective amount” is that amount of an isolated GH16 polypeptide which increases the rate or the extent of degradation of a cellulosic substrate by a cellulose-degrading composition compared to an otherwise equivalent composition lacking an isolated GH16 polypeptide under substantially equivalent reaction conditions including, but not limited to, pH, temperature, time of reaction, and dosage of the enzyme composition per gram of cellulose. For example, an effective amount of an isolated GH16 polypeptide is the amount which, when combined with one or more CBH or EG enzyme, increases the rate or extent of cellulose degradation relative to an otherwise equivalent mixture comprising the same one or more CBH or EG enzyme but lacking the isolated GH16 polypeptide under substantially equivalent reaction conditions.

An effective amount of isolated GH16 polypeptide in the cellulose-degrading enzyme composition may be from about 5 wt % to about 50 wt % of the combined weight of the at least one or more cellobiohydrolase (CBH) enzymes or endoglucanase (EG) enzymes and the isolated GH16 polypeptide. For example, the effective amount of isolated GH16 polypeptide in the cellulose-degrading enzyme composition may be 5 wt %, 10 wt %, 15 wt %, 20 wt %, 25 wt %, 30 wt %, 35 wt %, 40 wt %, 45 wt %, 50 wt %, or any amount therebetween, of the combined weight of the at least one or more cellobiohydrolase (CBH) enzymes or endoglucanase (EG) enzymes and the isolated GH16 polypeptide.

By “isolated GH16 polypeptide” it is meant an enzyme preparation comprising a GH16 polypeptide and no more than 10% of polypeptides with which the GH16 polypeptide is naturally associated. For example, the enzyme preparation may comprise a GH16 polypeptide and no more than 10%, 8%, 6%, 4%, 2%, 1%, 0%, or any amount therebetween, of polypeptides with which it is naturally associated. The isolated GH16 polypeptide of the present invention may be produced by a genetically modified microbe containing an isolated nucleotide encoding a GH16 polypeptide. For example, an isolated GH16 polypeptide may be an endogenous or heterologous GH16 polypeptide produced by a genetically modified microbe.

The term “cellulose-degrading enzyme” (also “cellulase enzyme” or “cellulase”) broadly refers to enzymes that catalyze the hydrolysis of the beta-1,4-glucosidic bonds joining individual glucose units in the cellulose polymer. Enzymatic degradation of cellulose involves the synergistic actions of endoglucanases (E.C. 3.2.1.4) and cellobiohydrolases (E.C. 3.2.1.91). Endoglucanases hydrolyze accessible glucosidic bonds in the middle of the cellulose chain, while cellobiohydrolases release cellobiose from these chain ends processively. Cellobiohydrolases are also referred to as exoglucanases.

The following definitions refer to classification of cellulase enzymes, hemicellulase enzymes, and related enzymes and proteins, as defined by the by the Joint Commission on Biochemical Nomenclature of the International Union of Biochemistry and Molecular Biology (Published in Enzyme Nomenclature 1992, Academic Press, San Diego, Calif., ISBN 0-12-227164-5; with supplements in Eur. J. Biochem. 1994, 223, 1-5; Eur. J. Biochem. 1995, 232, 1-6; Eur. J. Biochem. 1996, 237, 1-5; Eur. J. Biochem. 1997, 250; 1-6, and Eur. J. Biochem. 1999, 264, 610-650, each of which are incorporated herein by reference; also see: chem.qmul.ac.uk/iubmb/enzyme/) and to the Glycoside Hydrolase (GH) Families of cellulases and beta-glucosidases as defined by the CAZy system which is accepted as a standard nomenclature for Glycoside Hydrolase (GH) enzymes (Coutinho, P. M. & Henrissat, B., 1999, “Carbohydrate-active enzymes: an integrated database approach.”

In Recent Advances in Carbohydrate Bioengineering, H. J. Gilbert, G. Davies, B. Henrissat and B. Svensson eds., The Royal Society of Chemistry, Cambridge, pp. 3-12, which is incorporated herein by reference; also see www.cazy.org/Glycoside-Hydrolases.html) and is familiar to those skilled in the art.

Cellulases typically share a similar modular structure, which consists of one or more catalytic domain and one or more carbohydrate-binding modules (CBM) joined by flexible linker peptide(s). Most cellulases comprise at least one catalytic domain of GH Family 5, 6, 7, 8, 9, 12, 44, 45, 48, 51, 61 and 74.

In addition to the above CAZy system of nomenclature, cellobiohydrolases (CBH) and endoglucanases (EG) have been, and continue to be, identified by an earlier nomenclature system whereby each successive CBH or EG identified or isolated from a given source organism is numbered sequentially in the order of discovery (e.g., CBH1, CBH2, EG1, EG2, and so forth).

For the purposes herein, the following identifiers are considered equivalent:

| | CBH/EG | CAZy | SEQ ID |
|---|---|---|---|
| Enzyme | identifier | identifier | NO: |
| T. reesei cellobiohydrolase 1 | TrCBH1 | TrCel7A | 9 |
| T. reesei cellobiohydrolase 2 | TrCBH2 | TrCel6A | 10 |
| T. reesei endoglucanase 1 | TrEG1 | TrCel7B | 16 |
| T. reesei endoglucanase 2 | TrEG2 | TrCel5A | 11 |
| T. reesei beta-glucosidase 1 | TrBgl1 | TrCel3A | 12 |
| M. thermophila cellobiohydrolase 1a | MtCBH1a | MtCel7A | 20 |
| M. thermophila cellobiohydrolase 2b | MtCBH2b | MtCel6B | 23 |
| M. thermophila endoglucanase 1b | MtEG1b | MtCel7D | 24 |
| M. thermophila endoglucanase 2a | MtEG2 | MtCel5A | 22 |
| M. thermophila beta-glucosidase 1 | MtBgl1 | MtCel3A | 21 |

The one or more CBH and EG enzymes, and the isolated GH16 polypeptide of the cellulose-degrading composition may comprise either a “native” or “wild-type” amino acid sequence—i.e., the amino acid sequence as found naturally in the source organism(s) from which they are obtained—or a modified amino acid sequence—i.e., an amino acid sequence containing one or more insertions, deletions or substitutions relative to the native amino acid sequence.

As defined herein, a “GH16 polypeptide” is a carbohydrate active enzyme comprising a Glycoside Hydrolase (GH) Family 16 catalytic domain. A GH16 polypeptide may exhibit from about 35% to about 100% amino acid sequence identity to the Gloeophyllum trabeum GH16 polypeptide (Gtra GH16 of SEQ ID NO: 3) or to the Leucosporidium scottii GH16 polypeptide (Lsco GH16 of SEQ ID NO: 4), or from about 50% to about 100% amino acid sequence identity the Coprinus cinereus GH16 polypeptide (Ccin GH16 of SEQ ID NO: 5), or from about 55% to about 100% amino acid sequence identity to the Phanerochaete chrysosporium GH16 polypeptide (Pchr GH16 of SEQ ID NO: 6), or from about 40% to about 100% amino acid sequence identity to the Geomyces pannorum GH16 polypeptide (Gpan GH16 of SEQ ID NO: 7), or any percent identity therebetween. For example, a GH16 polypeptide may be derived from any one of the organisms listed in Table 1 and demonstrates at least 35%, 40%, 50%, 60%, 70%, 80%, 85%, 90%, 95% or 100% identity, or any % identity therebetween, to SEQ ID NO: 3 or to SEQ ID NO: 4, at least 50%, 60%, 70%, 80%, 85%, 90%, 95% or 100% identity, or any % identity therebetween, to SEQ ID NO: 5, at least 55%, 60%, 70%, 80%, 85%, 90%, 95% or 100% identity, or any % identity therebetween, to SEQ ID NO: 6, at least 40%, 50%, 60%, 70%, 80%, 85%, 90%, 95% or 100% identity, or any % identity therebetween, to SEQ ID NO: 7. In other embodiments, the GH16 polypeptide may be one or more of SEQ ID NO: 3, SEQ ID NO: 4, SEQ ID NO: 5, SEQ ID NO: 6, and SEQ ID NO: 7. The GH16 polypeptide may be functionally linked to a carbohydrate binding module (CBM) with a high affinity for crystalline cellulose, such as a Family 1 cellulose binding domain. At the time of filing, over 2000 enzymes and proteins have been classified into GH Family 16. For example, additional GH16 polypeptides suitable for the cellulose-degrading enzyme composition of the present invention include the GH16 polypeptides of SEQ ID NO: 94 (from Myceliophthora thermophila, GenPept Acc. No. AE056822), SEQ ID NO: 95 (from Thielavia terrestris, GenPept Acc. No. AE063309), SEQ ID NO: 96 (from Botryotinia fuckeliana, GenPept Acc. No. CCD52829), SEQ ID NO: 97 (from Myceliophthora thermophila, GenPept Acc. No. AE054158), SEQ ID NO: 98 (from Botryotinia fuckeliana, GenPept Acc. No. 001551617), SEQ ID NO: 99 (from Thielavia terrestris, GenPept Acc. No. AE065858), SEQ ID NO: 100 (from Rhizopus orzyae, GenPept Acc. No. AAQ20798), SEQ ID NO: 101 (from Aspergillus nidulans, GenPept Acc. No. EEA66118), and SEQ ID NO: 102 (from Penicillium chrysogenum, GenPept. Acc. No. CAP91414).

TABLE 1
Sequence Identity of GH16 polypeptides to GtraGH16 (GH16 from Gloeophyllum trabeum), LscoGH16
(GH16 from Leucosporidium scottii), CcinGH16 GH16 from Coprinus cinereus), PchrGH16 (GH16 from
Phanerochaete chrysosporium), and GpanGH16 (GH16 from Geomyces pannorum).

| | | | | Identity with |
|---|---|---|---|---|
| SEQ ID | | | GenPept | SEQ ID NO 3: |
| NO: | Organism | Protein | Accession No. | (Gtra GH16) |
| 25 | Serpula lacrymans var. | Glycoside hydrolase family 16 protein | EGO3548.1 | 45.9 |
| | lacrymans S7.3 | | | |
| 28 | Postia placenta Mad-698- | Hypothetical protein | XP_002470721.1 | 40.8 |
| | R | POSPLDRAFT_116903 | | |
| 31 | Schizophyllum commune | Glycoside hydrolase family 16 protein | XP_003038066.1 | 40.8 |
| | H4-8 | | | |
| 30 | Laccaria bicolor S238N- | Glycoside hydrolase family 16 protein | XP_001876832.1 | 40.1 |
| | H82 | | | |
| 27 | Piriformospora indica | Related to endo-1,3(4)-beta | CCA69113.1 | 39.3 |
| | DSM 11827 | glucanase | | |
| 40 | Postia placenta Mad-698- | Hypothetical protein | XP_002475942.1 | 38.7 |
| | R | POSPLDRAFT_135021 | | |
| 26 | Schizophyllum commune | Glycoside hydrolase family 16 protein | XP_003028746.1 | 38.5 |
| | H4-8 | | | |
| 32 | Serpula lacrymans var. | Glycoside hydrolase family 16 protein | EGN98170.1 | 36.7 |
| | lacrymans S7.3 | | | |
| 33 | Coprinopsis cinerea | Glycosyl hydrolase family 16 protein | XP_001830602.2 | 35.8 |
| | okayama7#130 | | | |
| 35 | Laccaria bicolor S238N- | Glycoside hydrolase family 16 protein | XP_001875740.1 | 35.6 |
| | H82 | | | |
| 36 | Cryptococcus neoformans | Hypothetical protein | XP_567580.1 | 35.1 |
| | var. neoformans JEC21 | | | |
| 29 | Postia placenta Mad-698- | Hypothetical protein | XP_002472478.1 | 35.0 |
| | R | POSPLDRAFT_115945 | | |
| 38 | Serpula lacrymans var. | Glycoside hydrolase family 16 protein | EGO23746.1 | 34.9 |
| | lacrymans S7.9 | | | |
| 37 | Rhodotorula glutinis | Glycoside hdrolase family 16 protein | EGU13079.1 | 33.2 |
| | ATCC 204091 | | | |
| 34 | Moniliophthora | Hypothetical protein | XP_002392207.1 | 31.0 |
| | perniciosa FA553 | MPER_08251 | | |
| | | | GenPept | Identity with |
| | | | Accession | SEQ ID NO 4: |
| | Organism | Protein | Number | (Lsco GH16) |
| 37 | Rhodotorula glutinis | Glycoside hydrolase family 16 protein | EGU13079.1 | 65.4 |
| | ATCC204091 | | | |
| 32 | Serpula lacrymans var. | Glycoside hydrolase family 16 protein | EGN98170.1 | 40.3 |
| | lacrymans S7.3 | | | |
| 29 | Postia placenta Mad-698- | Hypothetical protein | XP_002472478.1 | 39.4 |
| | R | POSPLDRAFT_115945 | | |
| 39 | Schizophyllum commune | Glycoside hydrolase family 16 protein | XP_003033735.1 | 38.0 |
| | H4-8 | | | |
| 30 | Postia placenta Mad-698- | Hypothetical protein | XP_002470721.1 | 37.7 |
| | R | POSPLDRAFT_116903 | | |
| 27 | Piriformospora indica | Related to endo-1,3(4)- | CCA69113.1 | 37.6 |
| | DSM11827 | beta-glucanase | | |
| 35 | Laccaria bicolor S238N- | Glycoside hydrolase family | XP_001875740.1 | 36.7 |
| | H82 | 16 protein | | |
| 43 | Postia placenta Mad-698- | Hypothetical protein | XP_002472273 | 35.7 |
| | R | POSPLDRAFT_53931 | | |
| 40 | Postia placenta Mad-698- | Hypothetical protein | XP_002475942.1 | 35.5 |
| | R | POSPLDRAFT_135021 | | |
| 42 | Postia placenta Mad-698- | Predicted protein | XP_002471903.1 | 35.4 |
| | R | | | |
| 41 | Postia placenta Mad-698- | Hypothetical protein | XP_002472272.1 | 35.4 |
| | R | POSPLDRAFT_12923 | | |
| 31 | Schizophyllum commune | Glycoside hydrolase family 16 protein | XP_003038066.1 | 34.9 |
| | H4-8 | | | |
| 25 | Serpula lacrymans var. | Glycoside hydrolase family 16 protein | EGO03548.1 | 33.6 |
| | lacrymans S7.3 | | | |
| 30 | Laccaria bicolor S238N- | Glycoside hydrolase family 16 protein | XP_001876832.1 | 32.5 |
| | H82 | | | |
| 26 | Schizophyllum commune H4-8 | Glycoside hydrolase family 16 protein | XP_003028746.1 | 32.2 |
| | | | GenPept | Identity with |
| | | | Accession | SEQ ID NO 5: |
| | Organism | Protein | Number | (Ccin GH16) |
| 44 | Coprinopsis cinerea | Glycosyl hydrolase family 16 | XP_001837802.2 | 98.5 |
| | okayama7#130 | | | |
| 45 | Coprinopsis cinerea | Glycosyl hydrolase family 16 | XP_001830206.2 | 63.4 |
| | okayama7#130 | | | |
| 91 | Schizophyllum commune | Glycoside hydrolase family 16 protein | XP_003037278.1 | 56.4 |
| | H4-8 | | | |
| 46 | Laccaria bicolor S238N- | Glycoside hydrolase family 16 protein | XP_001873806.1 | 55.9 |
| | H82 | | | |
| 48 | Serpula lacrymans var. | Glycoside hydrolase family 16 protein | EGN96860.1 | 54.1 |
| | lacrymans S7.3 | | | |
| 49 | Laccaria bicolor S238N- | Glycoside hydrolase family 16 protein | XP_001878748.1 | 53.8 |
| | H82 | | | |
| 50 | Serpula lacrymans var. | Glycoside hydrolase family 16 protein | EGO22459.1 | 53.5 |
| | lacrymans S7.3 | | | |
| 51 | Serpula lacrymans var. | Glycoside hydrolase family 16 protein | EGO01749.1 | 51.8 |
| | lacrymans S7.3 | | | |
| 52 | Piriformospora indica | Related to mixed-linked | CCA74474.1 | 42.5 |
| | DSM 11827 | glucanase precursor MLG1 | | |
| 53 | Piriformospora indica | Related to mixed-linked | CCA72549.1 | 42.3 |
| | DSM 11827 | glucanase precursor MLG1 | | |
| 55 | Schizophyllum commune H4-8 | Glycoside hydrolase family 16 protein | XP_003037611.1 | 38.2 |
| 57 | Lentiula edodes | Putative glycoside | BAH80446.1 | 37.8 |
| | | hydrolase family16 | | |
| | | protein | | |
| 54 | Schizophyllum commune H4-8 | Glycoside hydrolase family 16 protein | XP_003037612.1 | 37.4 |
| 56 | Piriformospora indica | Related to endo-1,3(4)- | CCA73094.1 | 36.6 |
| | DSM 11827 | beta-glucanase | | |
| | | | GenPept | Identity with |
| | | | Accession | SEQ ID NO 6: |
| | Organism | Protein | Number | (Pchr GH16) |
| 58 | Phanerochaete | Putative laminarinase | BAC67687 | 81.2 |
| | chrysosporium | | | |
| 59 | Postia placenta Mad-698- | Endo-1,3(4)-beta | PC_002472256 | 63.4 |
| | R | glucanase-like protein | | |
| 61 | Serpula lacrymans var. | Glycoside hydrolase family 16 protein | EGN97297 | 61.6 |
| | lacrymans S7.3 | | | |
| 64 | Piriformospora indica | Related to endo-1,3(4)- | CCA75235 | 61.2 |
| | DSM 11827 | beta-glucanase | | |
| 65 | Postia placenta Mad-698- | Hypothetical endo-1,3(4)- | XP_002476652 | 60.0 |
| | R | beta glucanase from | | |
| | | glycoside hydrolase family 16 | | |
| 63 | Coprinopsis cinerea | Glycosyl hydrolase family 16 protein | XP_001840143 | 59.5 |
| | okayama7#130 | | | |
| 67 | Laccaria bicolor S238N- | Glycoside hydrolase family 16 protein | XP_001887475 | 59.1 |
| | H82 | | | |
| 66 | Laccaria bicolor S238N- | Glycoside hydrolase family 16 protein | XP_001882200 | 58.7 |
| | H82 | | | |
| 62 | Schizophyllum commune | Glycoside hydrolase family 16 protein | XP_003031202 | 58.6 |
| | H4-8 | | | |
| 68 | Serpula lacrymans var. | Glycoside hydrolase family 16 protein | EGO00838 | 58.5 |
| | lacrymans S7.3 | | | |
| 60 | Laccaria bicolor S238N- | Glycoside hydrolase family 16 protein | XP_001887071 | 57.6 |
| | H82 | | | |
| 70 | Schizophyllum commune | Glycoside hydrolase family 16 protein | XP_003035920 | 56.0 |
| | H4-8 | | | |
| 72 | Coprinopsis cinerea | Endo-1,3(4)-beta- | XP_001840141 | 55.3 |
| | okayama7#130 | glucanase | | |
| 71 | Laccaria bicolor S238N- | Glycoside hydrolase family 16 protein | XP_001887072 | 55.2 |
| | H82 | | | |
| 69 | Postia placenta Mad-298- | Hypothetical beta- | XP_002473184 | 48.7 |
| | R | glucanase from glycoside | | |
| | | hydrolase family GH16 | | |
| | | | GenPept | Identity with |
| | | | Accession | SEQ ID NO 7: |
| | Organism | Protein | Number | (Gpan GH16) |
| 74 | Botryotinia fuckeliana | Glycoside hydrolase family 16 protein | CCD44624.1 | 45.1 |
| 77 | Talaromyces stipitatus | Endo-1,3(4)-beta- | XP_002484108.1 | 45.1 |
| | ATCC 10500 | glucanase, putative | | |
| 85 | Neosartorya fischeri | Endo-1,3(4)-beta- | XP_001265139.1 | 43.9 |
| | NRRL 181 | glucanase, putative | | |
| 75 | Aspergillus flavus | Endo-1,3(4)-beta- | XP_002374505.1 | 43.6 |
| | NRRL3357 | glucanase, putative | | |
| 76 | Aspergillus oryzae RIB40 | Unnamed protein product | BAE57957.1 | 43.6 |
| 86 | Aspergillus fumigatus | GPI anchored endo-1,3(4)- | XP_755769.1 | 43.3 |
| | Af293 | beta-glucanase | | |
| 78 | Arthroderma otae CBS 113480 | 1,3(4)-beta-glucanase | XP_002846056.1 | 43.3 |
| 79 | Trichophyton equinum | 1,3(4)-beta-glucanase | EGE05182.1 | 42.9 |
| | CBS 127.97 | | | |
| 81 | Trichophyton verrucosum | Endo-1,3(4)-beta- | XP_003022319.1 | 42.9 |
| | HKI 0517 | glucanase, putative | | |
| 80 | Trichophyton tonsurans | Endo-1,3(4)-beta- | EGE00369.1 | 42.9 |
| | CBS 112818 | glucanase | | |
| 82 | Trichophyton rubrum | Endo-1,3(4)-beta- | XP_003233194.1 | 42.6 |
| | CBS 118892 | glucanase | | |
| 84 | Arthroderma benhamiae | Endo-1,3(4)-beta- | XP_003017637.1 | 42.6 |
| | CBS 112371 | glucanase, putative | | |
| 83 | Paecilomyces sp. J18 | Beta-1,3-1,4-glucanase | ADK55597.1 | 42.3 |
| 73 | Glarea lozoyensis 74030 | Putative endo-1,3(4)-beta- | EHL01813.1 | 41.8 |
| | | glucanase | | |
| 87 | Artheroderma gypseum | 1,3(4)-beta-glucanase | XP_003171497.1 | 41.4 |
| | CBS 118893 | | | |

Sequence identity can be readily determined by alignment of the amino acids of the two sequences, either using manual alignment, or any sequence alignment algorithm as known to one of skill in the art, for example but not limited to, BLAST algorithm (BLAST and BLAST 2.0; Altschul et al., 1977, Nuc. Acids Res. 25:3389-3402; and Altschul et al., 1990, J. Mol. Biol. 215:403-410), the algorithm disclosed by Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). In the case of conducting BLAST alignments and sequence identity determinations for cellulase enzymes, only the amino acid sequences comprising the catalytic domains are considered.

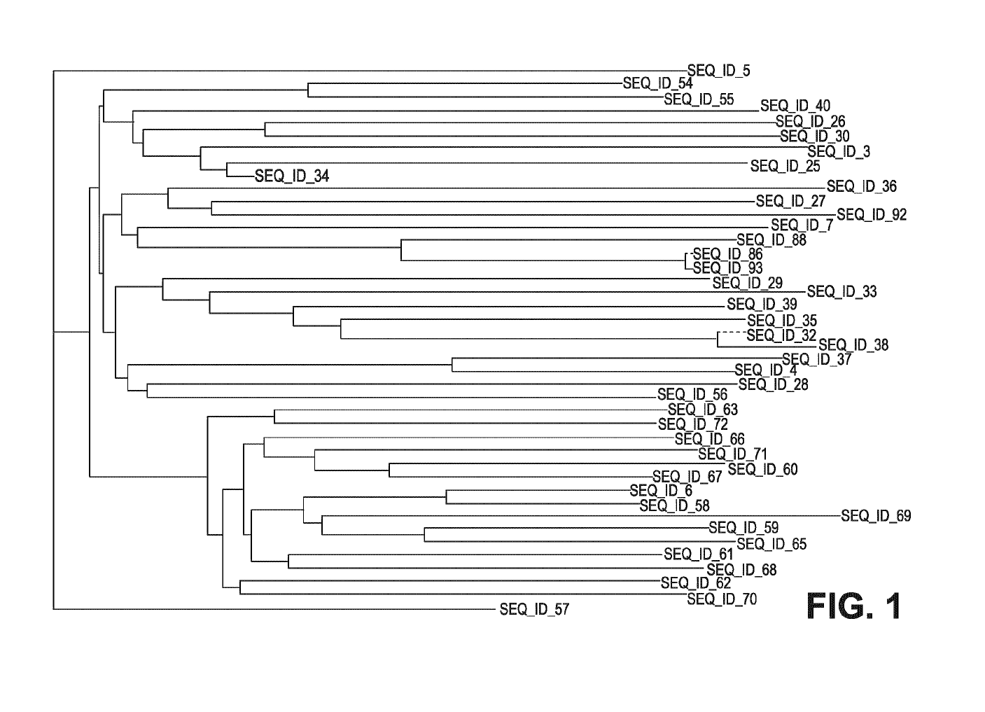

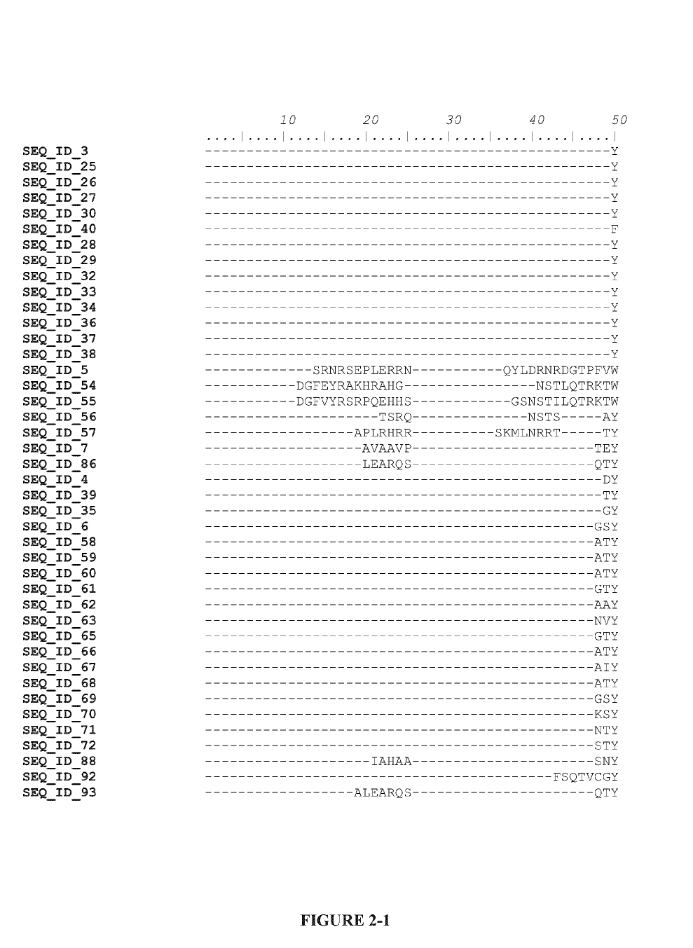

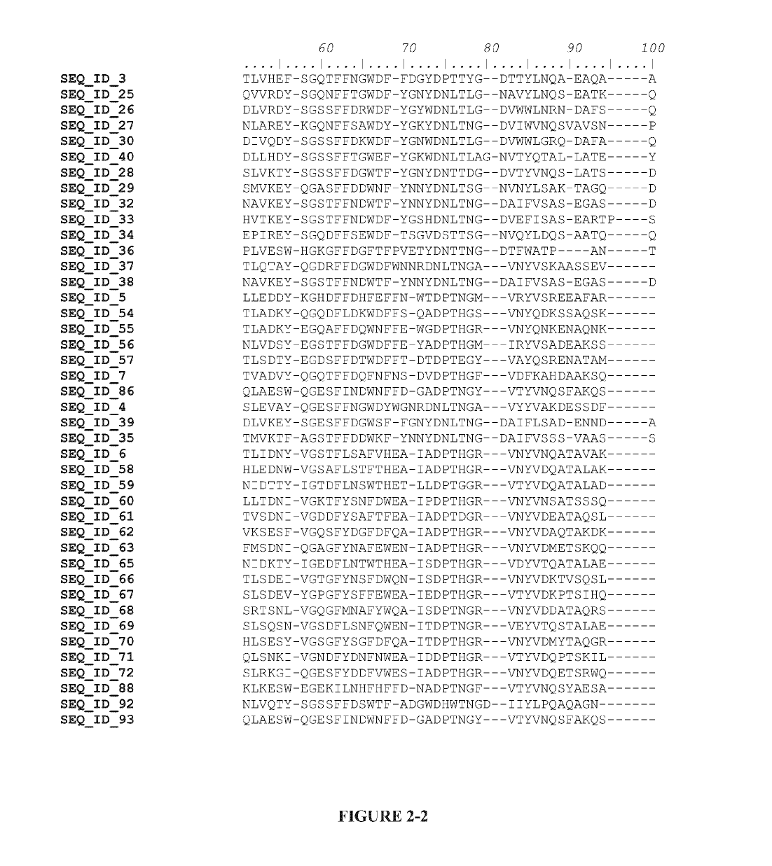

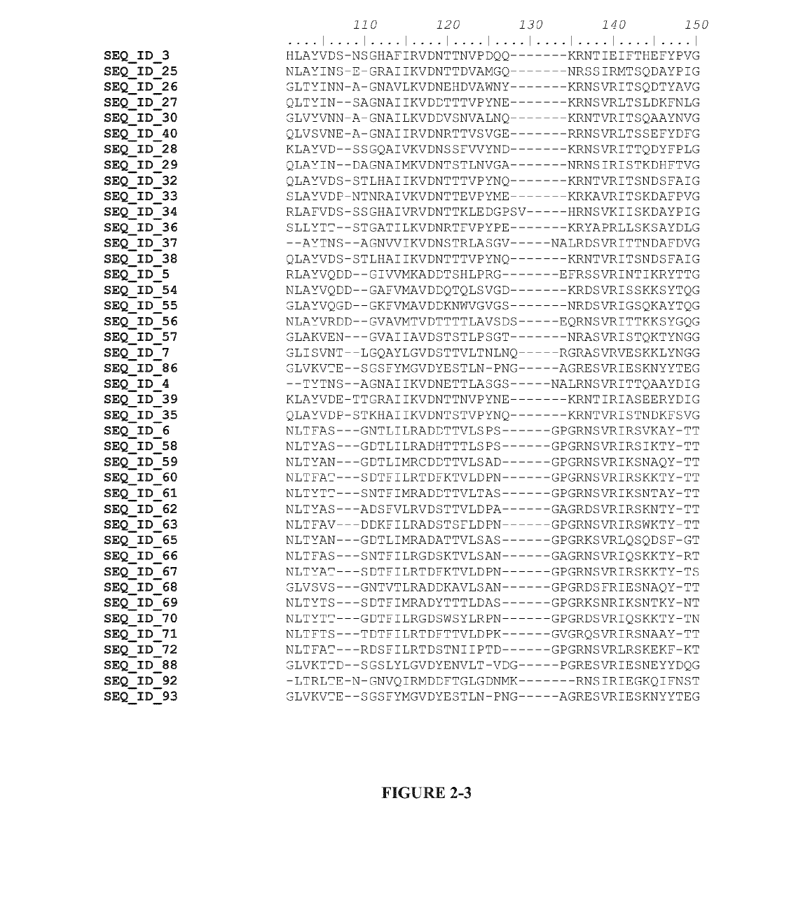

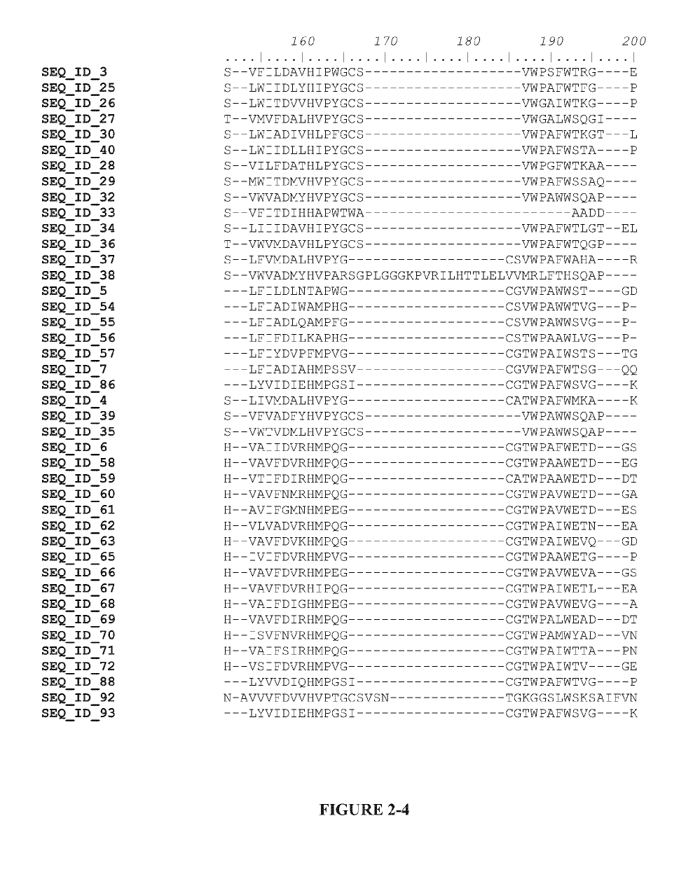

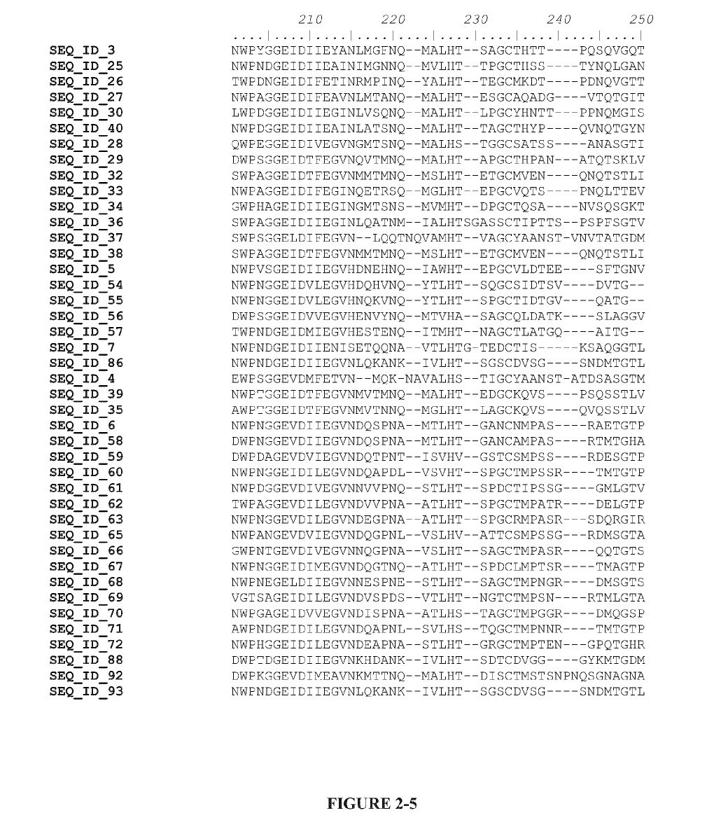

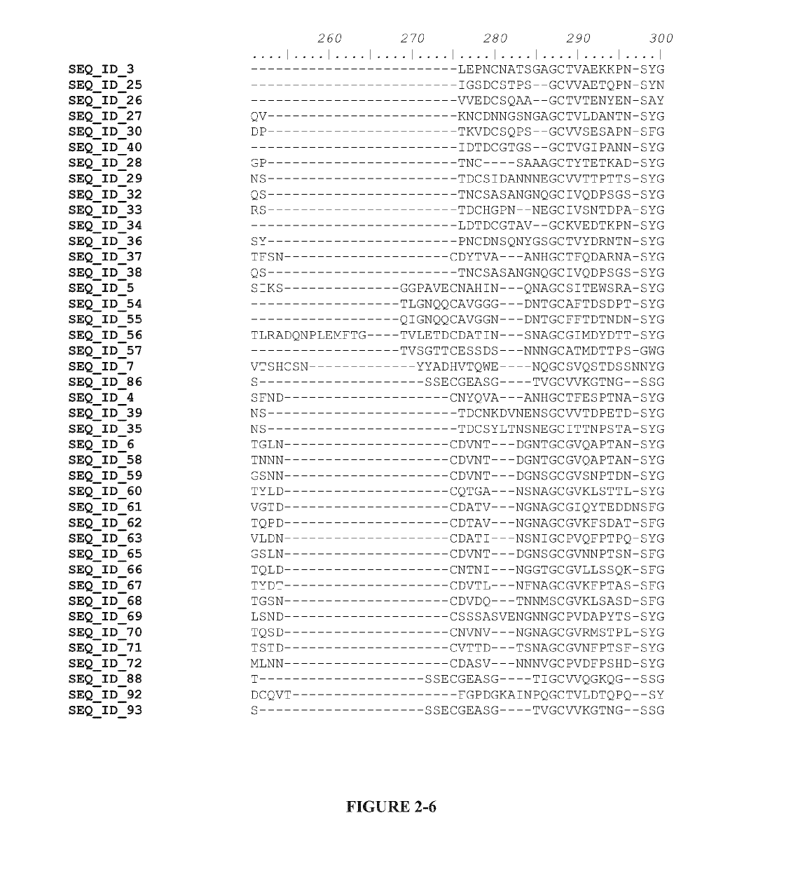

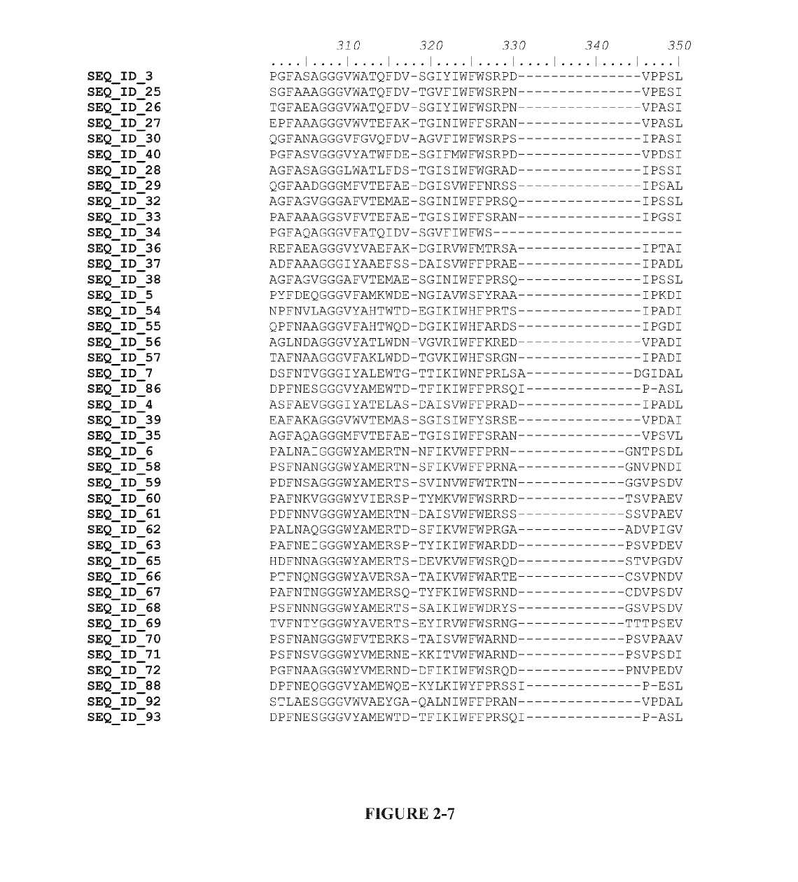

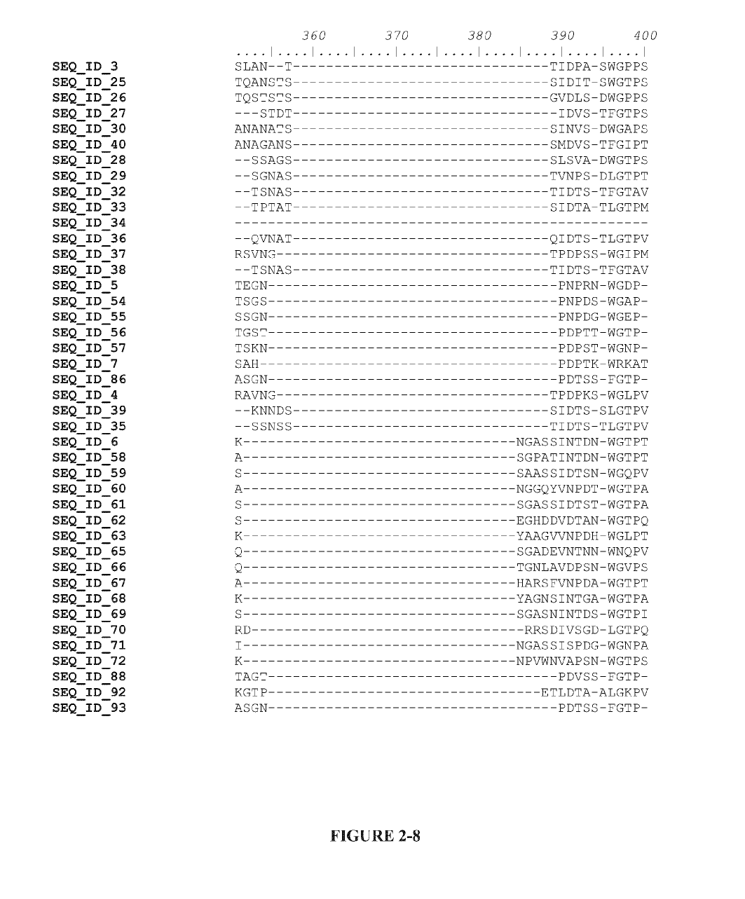

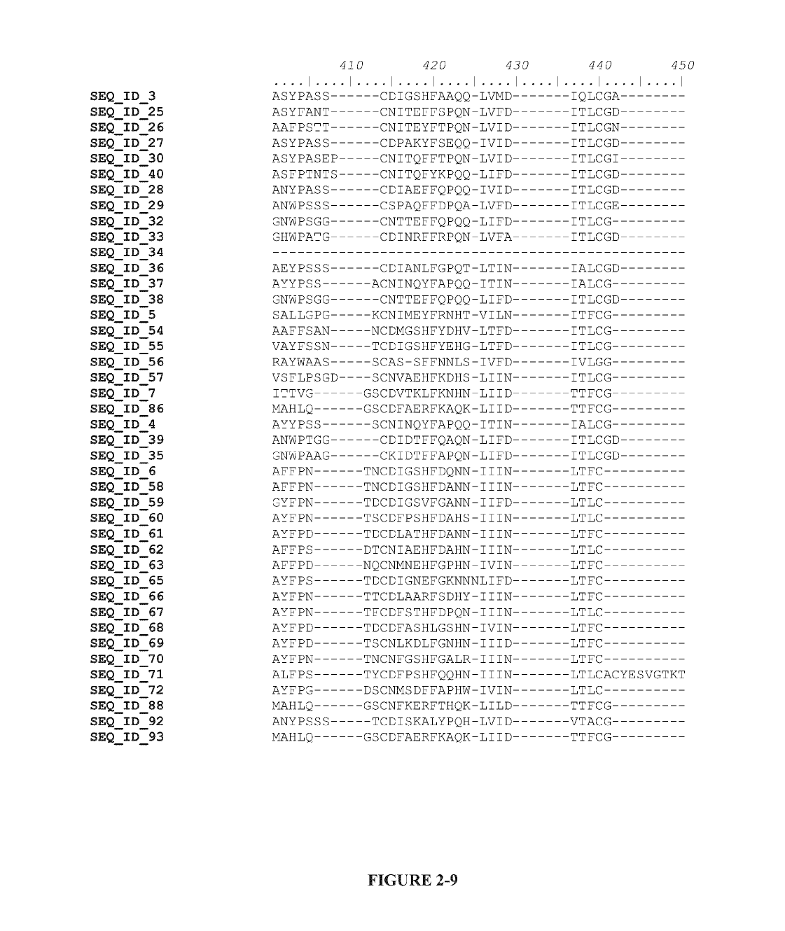

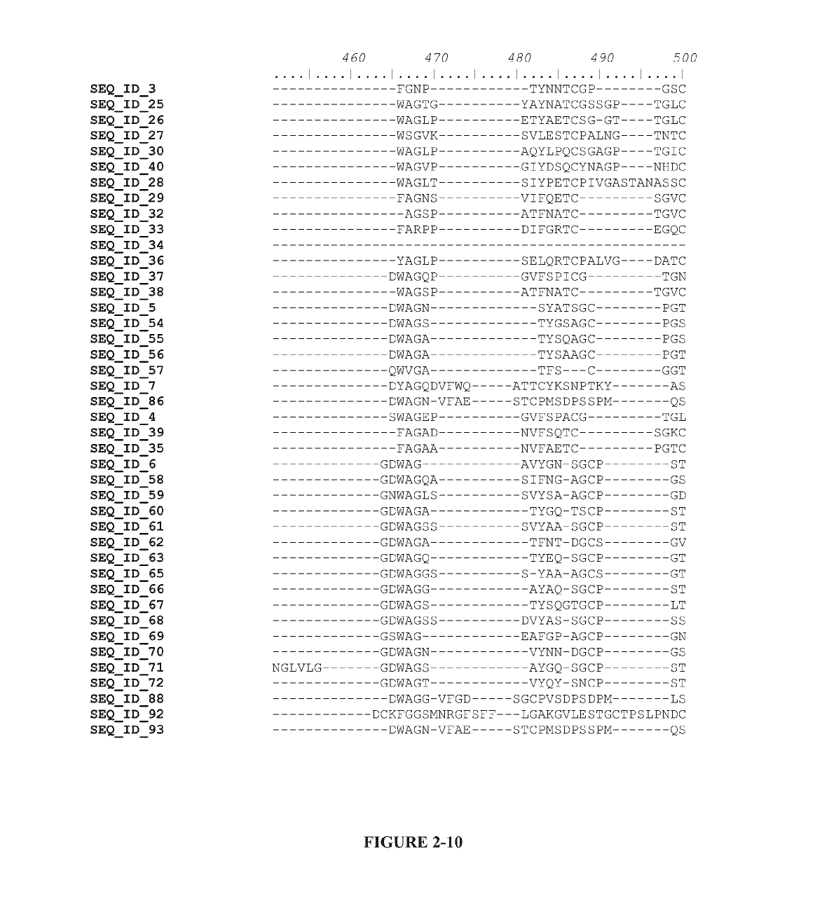

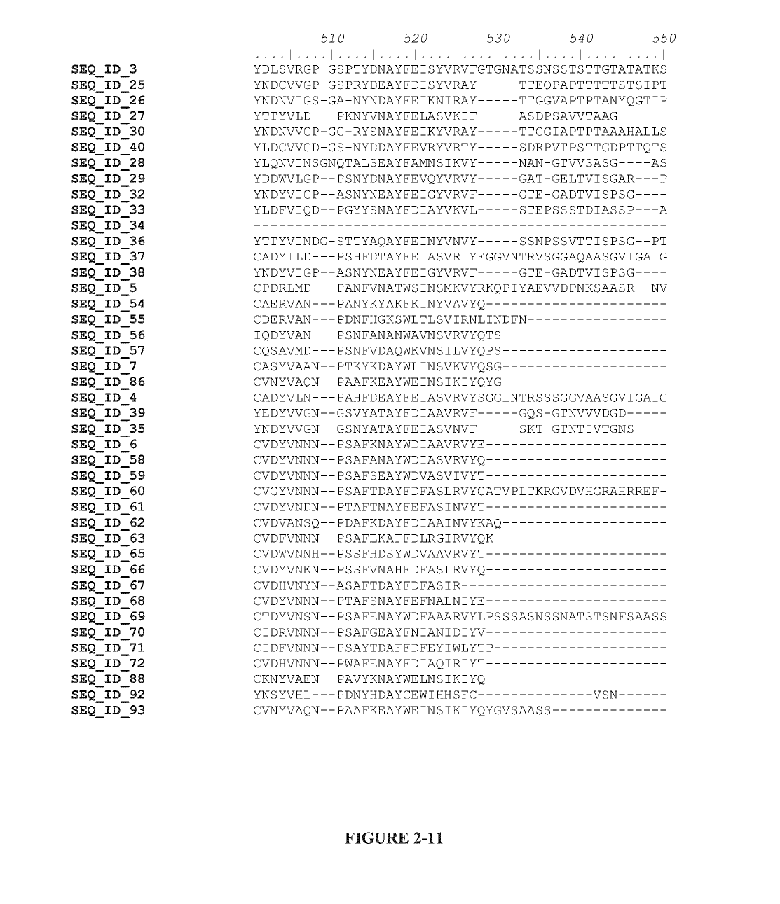

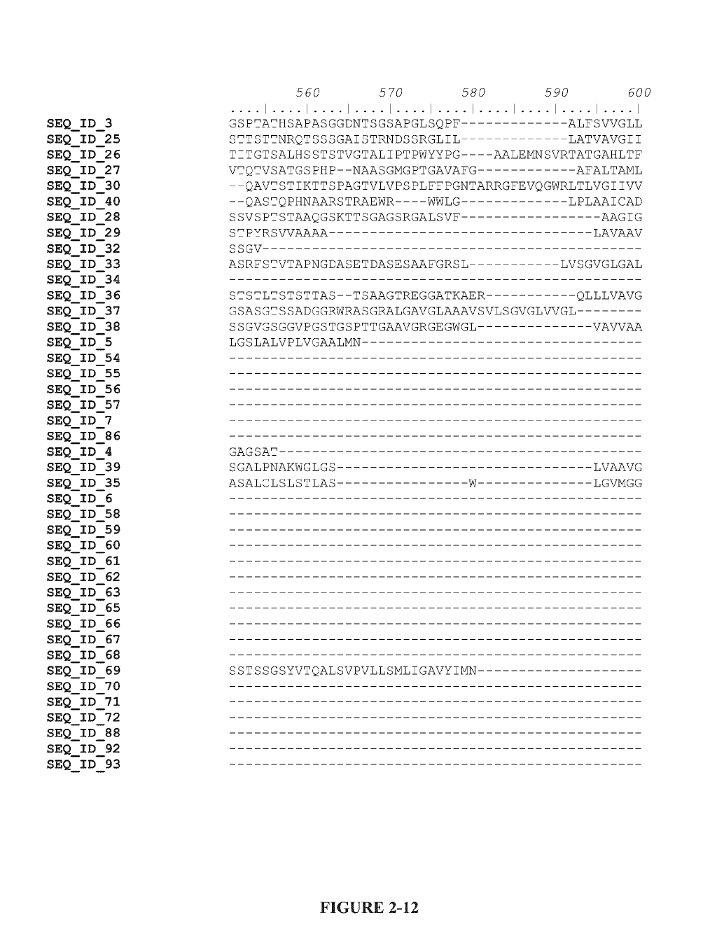

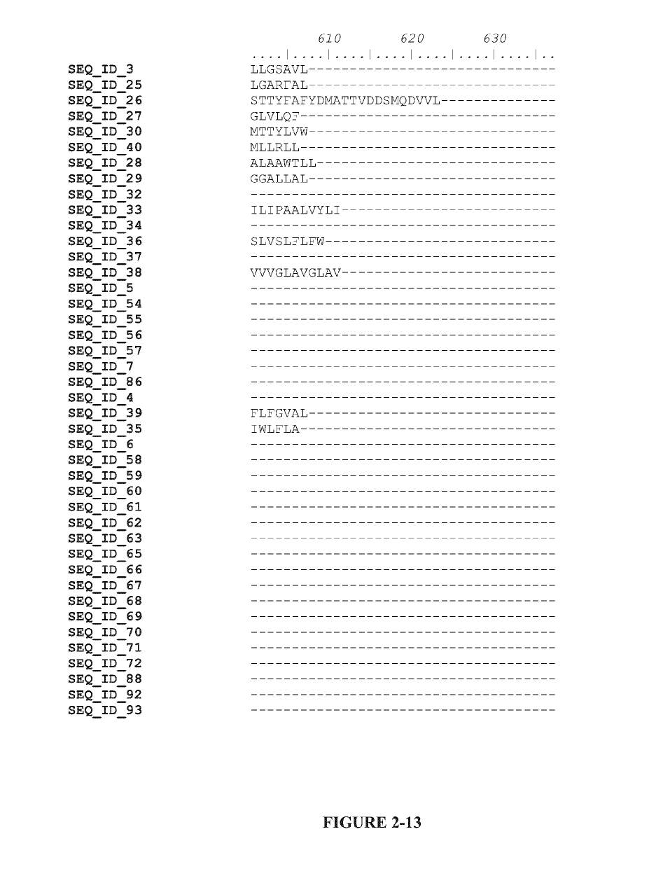

As shown in FIG. 1, the amino acid sequence of the GH16 polypeptides of SEQ ID NO: 3, 4, 5, 6, 7 which are natively produced by, respectively, Gloeophyllum trabeum, Leucosporidium scottii, Coprinus cinereus, Phanerochaete chrysosporium, and Geomyces pannorum, define a phyogenetically related group of source organisms for isolated GH16 polypeptide. This group includes Gloeophyllum trabeum, Geomyces pannorum, Coprinus cinereus, Leucosporidium scottii, Phanerochaete chrysosporium, Schizophylum commune, Laccaria bicolor, Serpula lacrymans, Piriformospora indica, Postia placenta, Aspergillus fumigatus, Aspergillus nidulans, Rhodotorula glutinis, Lentiula edodes, Cryptococcus neoformans, and taxonomic equivalents thereof. An amino acid sequence alignment of the GH16 polypeptides from these source organisms is provided in FIG. 2.

A GH16 polypeptide may exhibit one or more of the following hydrolytic activities: xyloglucan:xyloglucosyltransferase (EC 2.4.1.207), keratan-sulfate endo-1,4-beta-galactosidase (EC 3.2.1.103), endo-1,3-beta-glucanase (EC 3.2.1.39), endo-1,3(4)-beta-glucanase (EC 3.2.1.6), licheninase (EC 3.2.1.73), beta-agarase (EC 3.2.1.81), κ-carrageenase (EC 3.2.1.83), xyloglucanase (EC 3.2.1.151), endo-beta-1,3-galactanase (EC 3.2.1.-), and beta-porphyranase (EC 3.2.1.178).

In some embodiments of the present invention, the one or more CBH enzyme in the cellulose-degrading enzyme mixture is a member of GH Family 7. A “GH7 cellobiohydrolase” is a carbohydrate active enzyme comprising a Glycoside Hydrolase (GH) Family 7 catalytic domain classified under EC 3.2.1.91. A GH7 cellobiohydrolase may exhibit from about 60% to about 100% amino acid sequence identity to the catalytic domain (amino acids 1-436) of the Trichoderma reesei Cel7A enzyme (SEQ ID NO: 9) or to the catalytic domain (amino acids 1-438) of the Myceliophthora thermophila Cel7A enzyme (SEQ ID NO: 20). For example, the GH7 cellobiohydrolase may be derived from any one of the organisms listed in Table 2 and demonstrate at least 60%, 70%, 80%, 85%, 90%, 95% or 100% identity, or any % identity therebetween, to amino acids 1-436 of SEQ ID NO: 9 or to amino acids 1-438 of SEQ ID NO: 20. The GH7 cellobiohydrolase may be functionally linked to a carbohydrate binding module (CBM) with a high affinity for crystalline cellulose, such as a Family 1 cellulose binding domain.

TABLE 2
Sequence Identity of GH7 cellobiohydrolase Enzymes to Trichoderma reesei
Cel7A and to Myceliophthora thermophila CBH1a

| | | GenPept | |
|---|---|---|---|
| Organism | Protein | Accession | |
| | | | % Identity with |
| | | | amino acids 1-436 |
| | | | of SEQ ID NO: 9 |
| | | | (TrCel7A) |
| Hypocrea koningii G-39 | Cellobiohydrolase (Cbh1) - | CAA49596 | 100.0 |
| | Cel7A | | |
| Trichoderma viride AS | Cellobiohydrolase I | AAQ76092 | 99.3 |
| 3.3711 | | | |
| Trichoderma viride | 1,4-beta-D-glucan | CAA37878 | 96.1 |
| | Cellobiohydrolase | | |
| Trichoderma harzianum | Cellobiohydrolase | AAF36391 | 81.9 |
| Aspergillus niger CBS | 1,4-beta-D-glucan | AAF04491 | 65.5 |
| 513.88 | cellobiohydrolase A | | |
| | precursor | | |
| Talaromyces emersonii | Cellobiohydrolase 1- Cel7A | AAL33603 | 65.0 |
| Thermoascus aurantiacus | Cellobiohydrolase Precursor | AAW27920 | 64.6 |
| var. levisporus | | | |
| Aspergillus oryzae KBN616 | Cellobiohydrolase C | BAC07255 | 63.8 |
| Thermoascus aurantiacus | Cellobiohydrolase Precursor | AAL16941 | 63.2 |
| Penicillium occitanis | Cellobiohydrolase I | AAT99321 | 63.2 |
| Penicillium funiculosum | xylanase/cellobiohydrolase | CAC85737 | 63.0 |
| Cryphonectria parasitica | Cellobiohydrolase | AAB00479 | 62.6 |
| EP155 | | | |
| Acremonium thermophilum | Cellulose 1,4-beta- | CAM98445 | 62.5 |
| ALKO4245 | cellobiosidase | | |
| Aspergillus niger CBS | 1,4-beta-D-glucan | AAF04492 | 61.8 |
| 513.88 | cellobiohydrolase B | | |
| | precursor | | |
| Neurospora crassa OR74A | Exoglucanase 1 Precursor | EAA33262 | 61.0 |
| Penicillium chrysogenum | Exo-cellobiohydrolase | AAV65115 | 60.8 |
| FS010 | | | |
| Aspergillus oryzae RIB 40 | Cellobiohydrolase D | BAE61042 | 60.4 |
| | | | % Identity with |
| | | | amino acids 1-438 |
| | | | of SEQ ID NO: 20 |
| | | | (MtCBH1a) |
| Chaetomium thermopjilum | Exoglucanse-like protein | EGS21251.1 | 82.0 |
| var thermophilum DSM 1495 | | | |
| Thielavia terrestris NRRL | Glycoside hydrolase family | XP_003653508.1 | 81.0 |
| 8126 | 7 protein | | |
| Podospora anserina S mat+ | Hypothetical protein | XP_001903333.1 | 76.6 |
| Sordaria marcrospora k- | Hypothetical protein | XP_003346627.1 | 76.1 |
| hell | SMAC_03724 | | |
| Neurospora crassa OR74A | Exoglucanase 1 presursor | XP_962498.1 | 75.0 |
| Neurospora tetrasperma | Hypothetical protein | EGO59611.1 | 75.0 |
| FGSC 2508 | NEUTE1DRAFT_145583 | | |
| Acremonium thermophilum | Cellulose 1,4-beta- | CAM98445.1 | 75.0 |
| | cellobiosidase | | |
| Sordaria macrospora k-hell | Hypothetical protein | XP_003350595 | 70.6 |
| | SMAC_07912 | | |
| Gibberella avenacea | Exoglucanase type C | AAS82857.1 | 69.6 |
| | precursor | | |
| Gibberella pulicaris | Exoglucanase type C | AAS82858.1 | 69.0 |
| | precursor | | |
| Gibberella zeae | Glycoside hydrolase 7 | AAR02398.1 | 68.8 |
| Fusarium venenatum | Exoglucanase type C | AAX60001.1 | 68.8 |
| | precursor | | |
| Fusarium oxysporum | Putative exoglucanase type | P46238.1 | 67.9 |
| | C | | |

GH7 catalytic domains are distinguished by a beta-jelly roll core structure, with much of the protein in random coil held together by disulfide bonds. GH7 catalytic domains of CBH enzymes have peptide loops that cover the active site cleft, turning it into a closed tunnel that channels a cellulose chain past the active site residues and enables high processivity (Kleywegt et al., 1997, J. Mol Biol. 272:383). All Family 7 cellulases comprise two glutamic acid (E) residues which may serve as catalytic residues. These glutamic acid residues are found at positions 212 and 217 of Trichoderma reesei Cel7A (Divine, et al., 1998, J. Mol. Biol. 275: 309-325). The homologous glutamic acids in the M. thermophila CBH1a are found at positions 213 and 218.

In some embodiments of the present invention, the one or more CBH enzyme in the cellulose-degrading enzyme mixture is a member of GH Family 6. A “GH6 cellobiohydrolase” is a carbohydrate active enzyme comprising a Glycoside Hydrolase (GH) Family 6 catalytic domain classified under EC 3.2.1.91. A GH6 cellobiohydrolase may exhibit from about 45% to about 100% amino acid sequence identity to amino acids 83-447 comprising the catalytic domain of the Trichoderma reesei Cel6A enzyme (SEQ ID NO: 10) or to the catalytic domain (amino acids 118-432) of the Myceliophthora CBH2b enzyme (SEQ ID NO: 23). For example, the GH6 cellobiohydrolase enzyme may be derived from any one of the organisms listed in Table 3 and demonstrate at least 45%, 50%, 60%, 70%, 80%, 85%, 90%, 95% or 100% identity, or any % identity therebetween, to amino acids 83-447 of SEQ ID NO: 9 or to amino acids 118-432 of SEQ ID NO: 23. The GH6 cellobiohydrolase may be functionally linked to a carbohydrate binding module (CBM) with a high affinity for crystalline cellulase, such as a Family 1 cellulose binding domain.

TABLE 3
Sequence Identity of GH6 Cellobiohydrolase Enzymes to Trichoderma reesei
Cel6A and to Myceliophthora thermophila CBH2b

| | | GenPept | |
|---|---|---|---|
| Organism | Protein | Accession | |
| | | | % Identity with |
| | | | amino acids 83-447 |
| | | | of SEQ ID NO: 10 |
| | | | (TrCel6A) |
| Hypocrea koningii | cellobiohydrolase II (Cbh2) | AAK01367.1 | 98.9 |
| Trichoderma viride CICC 13038 | cellobiohydrolase II | AAQ76094.1 | 98.9 |
| | (CbhII; Cbh2) | | |
| Hypocrea koningii 3.2774 | cellobiohydrolase II | ABF56208.1 | 98.1 |
| | (Cbh2; CbhII) | | |
| Hypocrea koningii | cbh2 | ABG48766.1 | 97.8 |
| AS3.2774 | | | |
| Trichoderma parceramosum | cellobiohydrolase II (CbhII) | AAU05379.2 | 97.8 |
| Aspergillus nidulans FGSC | cellobiohydrolase (AN5282.2) | ABF50873.1 | 72.4 |
| A4 | | | |
| Aspergillus niger CBS | An12g02220 | CAK41068.1 | 72.4 |
| 513.88 | | | |
| Aspergillus oryzae RIB 40 | AO090038000439 | BAE64227.1 | 67.8 |
| Aspergillus niger CBS | An08g01760 | CAK39856.1 | 67.7 |
| 513.88 | | | |
| Acremonium cellulolyticus | cellobiohydrolase II (Acc2) | AAE50824 | 67.3 |
| Y-94 | | | |
| Talaromyces emersonii | cellobiohydrolase II (CbhII) | AAL78165.2 | 66.8 |
| Gibberella zeae K59 | Cel6 - Cel6 | AAQ72468.1 | 66.1 |
| Fusarium oxysporum | endoglucanase B | AAA65585.1 | 66.1 |
| Neurospora crassa OR74A | NCU09680.1 (64C2.180) | CAD70733.1 | 65.9 |
| Aspergillus nidulans FGSC | AN1273.2 | EAA65866.1 | 65.5 |
| A4 | | | |
| Magnaporthe grisea 70-15 | MG05520.4 | XP_360146.1 | 65.4 |
| Chaetomium thermophilum | cellobiohydrolase (Cbh2) | AAW64927.1 | 65.0 |
| CT2 | | | |
| Humicola insolens | avicelase 2 (Avi2) | BAB39154.1 | 63.7 |
| Cochliobolus | cellobiohydrolase II (CEL7) | AAM76664.1 | 59.6 |
| heterostrophus C4 | | | |
| Agaricus bisporus D649 | cellobiohydrolase II | AAA50607.1 | 57.7 |
| | (Cel3; Cel3A) | | |
| Polyporus arcularius 69B- | cellobiohydrolase II (Cel2) | BAF80327.1 | 57.1 |
| 8 | | | |
| Lentinula edodes Stamets | cellulase - Cel6B | AAK95564.1 | 56.3 |
| CS-2 | | | |
| Lentinula edodes L54 | cellobiohydrolase (CbhII-1) | AAK28357.1 | 56.0 |
| Malbranchea cinnamomea | unnamed protein product | CAH05679.1 | 54.9 |
| Phanerochaete | cellobiohydrolase II | AAB32942.1 | 54.9 |
| chrysosporium | | | |
| Volvariella volvacea | cellobiohydrolase II-I | AAT64008.1 | 53.8 |
| | (CbhII-I) | | |
| Chrysosporium | cellobiohydrolase (EG6; CBH | AAQ38151.1 | 49.5 |
| lucknowense | II) - Cel6A | | |
| Pleurotus sajor-caju | cellobiohydrolase II | AAL15037.1 | 47.2 |
| Trametes versicolor | ORF | AAF35251.1 | 47.0 |
| Neurospora crassa OR74A | NCU03996.1 | XP_323315.1 | 46.8 |
| | | | % Identity with |
| | | | amino acids 118-432 |
| | | | of SEQ ID NO: 23 |
| | | | (MtCBH2b) |
| Chaetomium thermophilum | Cellobiohydrolase family 6 | AAY88915.1 | 83.8 |
| Humicola insolens | Exoglucanase 6A | Q9C1S9.1 | 80.3 |
| Neurospora tetrasperma | Hypothetical protein | EGO61500.1 | 79.3 |
| FGSC 2508 | NEUTE1DRAFT_77549 | | |
| Neurospora crassa OR74A | Exoglucanase 2 precursor | XP_96-770.1 | 79.3 |
| Thielavia terrestris NRRL | Glycoside hyfrolase family 6 | XP_0036485846.1 | 79.0 |
| 8126 | protein | | |
| Chaetomium globosum | Hypothetical protein | XP_001226029.1 | 78.7 |
| CNS 148.51 | CHGG_10762 | | |
| Podospora anserina S | Hypothetical protein | XP_0019031730.1 | 75.8 |
| mat+ | | | |
| Sordaria macrospora k- | Hypothetical protein | XP_003346794.1 | 75.5 |
| hell | SMAC_05052 | | |
| Aspergillus fumigatus | Cellobiohydrolase | XP_748511.1 | 69.5 |
| Af293 | | | |
| Magnaporthe oryzae 70-15 | Hypothetical protein | XP_360146.1 | 69.5 |
| | MGG_05520 | | |
| Nectria haematococca | Hypothetical protein | XP_003049522.1 | 68.9 |
| mpVI 77-13-4 | NECHADRAFT_73991 | | |
| Phialophora sp. | Cellobiohydrolase II | ADZ99361.1 | 68.5 |
| CGMCC3328 | | | |
| Hypocrea jecorina | Cellobiohydrolase II | ADC83999.1 | 66.4 |
| Hypocrea rufa | Cellobiohydrolase II | AAQ76094.1 | 66.4 |
| Verticillium dahliae | Exoglucanase-6A | EGY16046.1 | 65.7 |
| VdLs.17 | | | |

All GH Family 6 cellulases comprise two aspartic acid (D) residues which may serve as catalytic residues. These aspartic acid residues are found at positions 175 and 221 of Trichoderma reesei Cel6A (SEQ ID NO: 10). The homologous glutamic acids in the M. thermophila CBH2b (SEQ ID NO: 23) are found at positions 213 and 218. GH Family 6 cellulases also share a similar three dimensional structure: an alpha/beta-barrel with a central beta-barrel containing seven parallel beta-strands connected by five alpha-helices.

In some embodiments of the present invention, the one or more EG enzyme in the cellulose-degrading enzyme composition is a member of GH Family 7. A “GH7 endoglucanase” is defined as a carbohydrate active enzyme comprising a GH Family 7 catalytic domain classified under EC 3.2.1.4. A GH7 endoglucanase may exhibit about 48% to about 100% amino acid sequence identity to amino acids 1-374 comprising the catalytic domain of the Trichoderma reesei Cel7B enzyme (SEQ ID NO: 16) or from about 65% to 100% identity to amino acids 30-390 comprising the catalytic domain of the Myceliophthora thermophila EG1b enzyme (SEQ ID NO: 24). For example, the GH7 endoglucanase may be obtained or derived from any one of the organisms listed in Table 4 and demonstrate at least about 48%, 50%, 60%, 70%, 80%, 85%, 90%, 95% or 100% identity, or any % identity therebetween, to amino acids 1-374 of SEQ ID NO: 16 or demonstrate at least about 65%, 70%, 75%, 80%, 85%, 90%, 95%, or 100% identity, or any % identity therebetween, to amino acids 30-390 of SEQ ID NO: 24 The GH7 endoglucanase may be functionally linked to a carbohydrate binding module (CBM) with a high affinity for crystalline cellulase, such as a Family 1 cellulose binding domain.

TABLE 4
Sequence Identity of GH7 Endoglucanases to Trichoderma reesei
Cel7B and to Myceliophthora thermophila EG1b

| | | GenPept | |
|---|---|---|---|
| Organism | Protein | Accession | |
| | | | % Identity with |
| | | | amino acids 1-374 |
| | | | of SEQ ID NO: 16 |
| | | | (TrCel7B) |
| Trichoderma viride AS | Endoglucanase I | AAQ21382 | 99.5 |
| 3.3711 | | | |
| Trichoderma | Endo-1,4-glucanase I | CAA43059 | 95.5 |
| longibrachiatum | | | |
| Hypocrea | Endoglucanase I | ABM90986 | 95.2 |
| pseudokoningii | | | |
| Penicillium decumbens 114-2 | Endoglucanase I | ABY56790 | 62.5 |
| Aspergillus oryzae RIB | Endo-1,4-glucanase | BAE66197 | 49.1 |
| 40 | | | |
| Aspergillus oryzae | Endo-1,4-glucanase (CelB) | BAA22589 | 48.9 |
| KBN616 | | | |
| Neurospora crassa | Endoglucanase EG-1 | EAA27195 | 48.7 |
| OR74A | precursor | | |
| Aspergillus nidulans | Endo-β-1,4-glueanase | EAA63386 | 47.9 |
| FGSC A4 | | | |
| Neurospora crassa | Hypothetical Protein | XP_324211 | 41.7 |
| OR74A | | | |
| | | | % Identity with |
| | | | amino acids 30-390 |
| | | | of SEQ ID NO: |
| | | | (MtEG1b) |
| Thielavia terrestris | Glycoside hydrolase family | XP_003653757.1 | 80.0 |
| NRRL 8126 | 7 protein | | |
| Chaetomim globosum | Hypothetical protein | XP_001229968.1 | 78.9 |
| CBS 148.51 | CHGG_03452 | | |
| Trichoderma virens | Glycoside hydrolase family | EHK18735.1 | 68.9 |
| Gv29-8 | 7 protein | | |
| Hypocrea orientalis | Endoglucanase I | AFD50194.1 | 67.5 |
| Hypocrea | Endoglucanase I | AEQ29501.1 | 67.3 |
| pseudokoningii | | | |
| Trichoderma | Endoglucanase I | ACZ34302.1 | 67.3 |
| longibrachiatum | | | |
| Trichoderma sp. SSL | Endoglucanase I | ACH68455.1 | 67.3 |
| Trichoderma reesei | Endoglucanase EG 1 | P07981.1 | 66.4 |
| Hypocrea rufa | Endoglucanase I | AAQ21382.1 | 66.4 |
| Aspergillus fumigatus | Endoglucanase | XP_747897.1 | 66.4 |
| Af293 | | | |
| Aspergillus terreus | Endoglucanase EG-1 | XP_001217291.1 | 66.2 |
| NIH2624 | precursor | | |
| Neosartorya fischeri | Endoglucanase, putative | XP_001257357.1 | 65.6 |
| NRRL 181 | | | |
| Trichoderma atroviride | Glycoside hydrolase family | EHK46214.1 | 65.3 |
| IMI 206040 | 7 protein | | |
| Aspergillus terreus | Beta-1,4-endoglucanase | ADR78837.1 | 65.1 |

In some embodiments of the present invention, the one or more EG enzyme in the cellulose-degrading enzyme composition is a member of GH Family 5. A “GH5 endoglucanase” is defined as a carbohydrate active enzyme comprising a Glycoside Hydrolase (GH) Family 5 catalytic domain classified under EC 3.2.1.4. A GH5 endoglucanase may exhibit about 40% to about 100% amino acid sequence identity, or more preferably about 48% to about 100% amino acid sequence identity, to amino acids 202 to 222 of the Trichoderma reesei Cel5A enzyme (SEQ ID NO: 11). This highly conserved region represented by amino acids 202-222 of SEQ ID NO: 11 includes one of the two catalytic glutamic acid residues that characterize GH Family 5. Alternatively, the GH5 endoglucanase may be obtained or derived from any one of the organisms listed in Table 5 and demonstrate at least about 40%, 50%, 60%, 70%, 80%, 85%, 90%, 95% or 100% identity, or any % identity therebetween, to amino acids 202-222 of SEQ ID NO: 11. A GH5 endoglucanase may also exhibit about 65% to about 100% amino acid sequence identity to amino acids 77-297 of the Myceliophthora thermophila EG2a enzyme (SEQ ID NO: 22). The GH5 endoglucanase may be obtained or derived from any one of the organisms listed in Table 5 and demonstrate at least about 65%, 70%, 75%, 80%, 85%, 90%, 95% or 100% identity, or any % identity therebetween, to amino acids 77-297 of SEQ ID NO: 22. The GH5 endoglucanase may be functionally linked to a carbohydrate binding module (CBM) with a high affinity for crystalline cellulase, such as a Family 1 cellulose binding domain.

TABLE 5
Sequence Identity of GH5 Endoglucanases to Trichoderma reesei
Cel5A and to Myceliophthora thermophila EG2

| | | GenPept | |
|---|---|---|---|
| Organism | Protein | Accession | |
| | | | % Identity with |
| | | | amino acids 202-222 |
| | | | of SEQ ID NO: 11 |
| | | | (TrCel5A) |
| Trichoderma viride | Endoglucanase | ABQ95572 | 100 |
| Trichoderma viride AS | Endoglucanase III | AAQ21383 | 100 |
| 3.3711 | | | |
| Trichoderma viride | Endo-1,4-glucanase II | BAA36216 | 100 |
| MC300-1 | | | |
| Trichoderma sp. C-4 | Endo-1,4-glucanase | AAR29981 | 92 |
| Phanerochaete | Endoglucanase - Cel5A | AAU12275 | 72 |
| chrysosporium | | | |
| Macrophomina phaseolina | Endo-1,4-glucanase | AAB03889 | 64 |
| Cryptococcus sp. S-2 | Carboxymethylcellulase | ABP02069 | 56 |
| Cryptococcus flavus | Carboxymethylcellulase | AAC60541 | 50 |
| Irpex lacteus MC-2 | Endoglucanase | BAD67544 | 48 |
| Hypocrea jecorina QM6a | Cel5B | AAP57754 | 48 |
| Macrophomina phaseolina | Endo-1,4-glucanase | AAB51451 | 44 |
| Thermoascus aurantiacus | EGI Precursor | AAL16412 | 44 |
| IFO 9748 | | | |
| Trametes hirsuta | Endoglucanase | BAD01163 | 44 |
| Aspergillis oryzae | Endo-1,4-glucanase | BAD72778 | 44 |
| | (CelE) | | |
| Talaromyces emersonii | Endo-1,4-glucanase | AAL33630 | 40 |
| Humicola grisea var. | Cellulase (Endo-1,4- | BAA12676 | 40 |
| thermoidea IFO9854 | glucanase 3) | | |
| Humicola insolens | Endo-1,4-glucanase IV | CAA53631 | 40 |
| Aspergillis kawachi | Endoglucanase C | BAB62319 | 40 |
| | (Cel5B) | | |
| Aspergillis nidulans | Endo-β-1,4-glucanase | ABF50848 | 40 |
| | | | % Identity with |
| | | | amino acids 77-297 |
| | | | of SEQ ID NO: |
| | | | (MtEG2)* |
| Chaetomium globosum | Hypothetical protein | XP_001220409 | 81.5 |
| CBS148.51 | CHGG_01188 | | |
| Sordaria marcospora k- | Hypothetical protein | XP_003352611.1 | 80.8 |
| hell | SMAC_01445 | | |
| Neurospora crassa | Endoglucanase 3 | XP_964159.1 | 79.7 |
| OR74A | | | |
| Neurospora tetrasperma | Putative cellulase | EGZ7679.1 | 79.7 |
| FGSC 2509 | precursor | | |
| Thielavia terrestris NRRL | Glycoside hydrolase | XP_003567015.1 | 77.7 |
| 8126 | family 5 protein | | |
| Humicola grisea var. | Cellulase | BAA12676.1 | 73.4 |
| thermoidea | | | |
| Humicola insolens | Endoglucanase 3 | Q12624.1 | 73.4 |
| Podospora anserina S | Hypothetical protein | XP_001912812.1 | 73.4 |
| mat+ | | | |
| Magnaporthe oryzae 70-15 | Endoglucanse 3 | EHA51103.1 | 72.0 |
| Glomerella graminicola | Cellulase | EFQ33605.1 | 70.7 |
| M1.001 | | | |
| Chaetomium thermophilum | Endoglucanase-like | EGS18971.1 | 69.6 |
| var. thermophilum DSM 1495 | protein | | |
| Nectria haematococca | Hypothetical protein | XP_003040869 | 68.0 |
| mpVI 77-13-4 | NECHADRAFT_97581 | | |
| Verticillium dahliae | Endoglucanase | EGY19676.1 | 67.6 |
| VdLs.17 | | | |
| Fusarium oxysporum | Hypothetical protein | EGU78866.1 | 66.6 |
| Fo5176 | FOXB_10604 | | |

GH Family 5 cellulases share a common (beta/alpha)₈-barrel fold and a catalytic mechanism resulting in a net retention of the anomeric sugar conformation. Glycoside hydrolase catalysis is driven by two carboxylic acids found on the side chain of glutamate residues (Ly and Withers, 1999, Annu. Rev. Biochem 68:487-622). In the GH Family 5 cellulase from T. reesei, residues E329 and E218 are the nucleophile and the acid/base respectively (Macarron et al., 1993, Biochem. J. 289:867-873). These two residues are highly conserved among family members (Wang et al., 1993, J. Bacteriol. 175(5):1293-1302).

In addition to the isolated GH16 polypeptide and the one or more CBH and/or EG enzymes(s), the cellulose-degrading enzyme composition may further comprise one or more additional enzymes and proteins that enhance the degradation of cellulose including, but not limited to, beta-glucosidases, proteins of Glycosyl Hydrolase Family 61, swollenin proteins, expansin proteins, and hemicellulases.

In some embodiments of the present invention, the one or more BGL enzyme is a member of GH Family 1 or GH Family 3. A “beta-glucosidase” (or BGL) is defined as any carbohydrate active enzyme from the GH Family 3 or GH Family 1 that is also classified under EC 3.2.1.21. The beta-glucosidase may be of fungal origin. For example, the beta-glucosidase may be a member of GH Family 3 and exhibit from about 42% to about 100% amino acid sequence identity to the Trichoderma reesei Cel3A enzyme (SEQ ID NO: 12) or from about 42% to about 100% amino acid sequence identity to the Myceliophthora thermophila Cel3A enzyme (SEQ ID NO: 21). A Family 3 beta-glucosidase may be obtained or derived from any one of the organisms listed in Table 6 and demonstrate at least about 40%, 50%, 60%, 70%, 80%, 85%, 90%, 95% or 100% identity, or any % identity therebetween, to SEQ ID NO: 12 or at least about 65%, 70%, 80%, 85%, 90%, 95% or 100% identity, or any % identity therebetween, to SEQ ID NO: 21.

TABLE 6
Sequence Identity of GH3 beta-glucosidases to Trichoderma reesei
Cel3A and to Myceliophthora thermophila Cel3A

| | | GenPept | |
|---|---|---|---|
| Organism | Protein | Accession | |
| | | | % Identity with |
| | | | SEQ ID NO: 12 |
| | | | (TrCe3A) |
| Trichoderma viride AS | b-D-glucoside glucohydrolase | AAQ76093.1 | 98.3 |
| 3.3711 | (Bgl1) | | |
| Phanerochaete | glucan b-1,3-glucosidase (Bgl) | BAB85988.1 | 52.6 |
| chrysosporium K-3 | | | |
| Phanerochaete | glucan1,3-b-glucosidase | AAC26489.1 | 52.6 |
| chrysosporium OGC101 | (CbgL) - Bgl1A | | |
| Thermoascus aurantiacus | b-glucosidase (Bg2; BGII) | AAY33982.1 | 45 |
| Thermoascus aurantiacus | b-1,4-glucosidase (Bgl2) | ABX56926.1 | 45 |
| var. levisporus | | | |
| Thermoascus aurantiacus | b-glucosidase (Bgl1; Bg1) | AAZ95587.1 | 44.3 |
| IFO 9748 | | | |
| Thermoascus aurantiacus | b-1,4-glucosidase (Bgl1) | ABX79552.1 | 44.3 |
| var. levisporus | | | |
| Aspergillus aculeatus F- | b-glucosidase 1 (Bgl1) | BAA10968.1 | 44.1 |
| 50 | | | |
| Aspergillus oryzae RIB 40 | b-glucosidase 5 | BAE57053.1 | 43.4 |
| | (Bgl5; AO090001000544) | | |
| Talaromyces emersonii | b-glucosidase - Cel3A | AAL69548.3 | 43.2 |
| Aspergillus fumigatus | b-glucosidase | EAL88289.1 | 43.1 |
| Af293 | (AFUA_1G05770; Afulg05770) | | |
| Aspergillus niger B1 | b-glucosidase/tannase | CAB75696.1 | 42.8 |
| | (Bgl1; BG3; BGs; SP188) | | |
| Phaeosphaeria avenaria | b-glucosidase (Bgl1) | CAB82861.1 | 42.7 |
| WAC1293 | | | |
| Aspergillus kawachii | b-glucosidase (BglA) | BAA19913.1 | 42.6 |
| ifo4308 | | | |
| Aspergillus niger CBS | An18g03570(Bgl1) | CAK48740.1 | 42.6 |
| 513.88 | | | |
| Aspergillus oryzae RIB 40 | b-glucosidase | BAE54829.1 | 42.3 |
| | (AO090009000356) | | |
| Aspergillus oryzae | b-glucosidase | CAD67686.1 | 42.2 |
| Periconia sp. BCC 2871 | b-glucosidase | ABX84365.1 | 41.9 |
| Hypocrea jecorina QM6a | b-glucosidase - Cel3B | AAP57755.1 | 41.5 |
| Coccidioides posadasii | b-glucosidase/exo-b-1,3- | AAF21242.1 | 41.4 |
| C735 | glucosidase (Bgl2) | | |
| Coccidioides posadasii | b-glucosidase (Bgl1) | AAB67972.1 | 40.4 |
| C735 | | | |
| Uromyces viciae-fabae | b-glucosidase (Bgl1) | CAE01320.1 | 39.8 |
| | | | % Identity with |
| | | | SEQ ID NO: |
| | | | (MtCel3A)* |
| Chaetomium globosum | Hypothetical protein | XP_001229937.1 | 86.1 |
| CBS 148.51 | CHGG_03421 | | |
| Thielavia terrestris NRRL | Glycoside hydrolase family 3 | XP_003655388.1 | 82.7 |
| 8126 | protein | | |
| Podospora anserine S | Hypothetical protein | XP_001907699.1 | 80.9 |
| mat+ | | | |
| Chaetomium | Beta-glucosidase | ABR57325.2 | 75.8 |
| thermophilum | | | |
| Neurospora crassa | Beta-glucosidase 1 precursor | XP_956104.1 | 75.0 |
| OR74A | | | |
| Sordaria macrospora k- | Hypothetical protein | XP_003345281.1 | 74.8 |
| hell | SMAC_045515 | | |
| Neurospora tetrasperma | Beta-glucosidase 1 precursor | EGO58510.1 | 74.6 |
| FGSC 2508 | | | |
| Magnaporthe grisea | Beta-glucosidase-like protein | AAX07690.1 | 73.9 |
| Magnaporthe oryzae 70-15 | Conserved hypothetical protein | XP_364427.2 | 70.4 |
| Botryotinia fuckeliana | Hypothetical protein | XP_001551395.1 | 70.2 |
| B05.10 | BC1G_10221 | | |
| Colletotrichum | Glycosyl hydrolase family 3 | CCF36272.1 | 69.3 |
| higginsianum | | | |
| Sclerotinia sclerotiorum | Beta-glucosidase 1 precrsor | XP_001591700.1 | 69.1 |
| 1980 | | | |
| Grosmannia clavigera | Beta-glucosidase 1 precursor | EFX03340.1 | 68.6 |
| kw1407 | | | |
| Glarea lozoyensis 74030 | Putative beta-glucosidase A | EHK9282.1 | 65.6 |
| Chaetomium | Beta-glucosidase-like protein | EGS20380.1 | 43.1 |
| thermophilum var | | | |
| thermophilum DSM1495 | | | |

The three dimensional structure of beta-D-glucan exo-hydrolase, a Family 3 Glycoside Hydrolase, was described by Varghese et al., 1994, Proc. Natl. Acad. Sci. USA 91(7):2785-2789. The structure was of a two domain globular protein comprising a N-terminal (a/13)₈ TIM-barrel domain and a C-terminal six-stranded beta-sandwich, which contains a beta-sheet of five parallel beta-strands and one antiparallel beta-strand, with three alpha-helices on either side of the sheet. The catalytic residues in the T. reesei Cel3A beta-glucosidase are D236 and E447, which are located within regions of very high amino acid sequence conservation within the Family 3 beta-glucosidases from amino acids 225-256 and 439-459, respectively.

Many polypeptides found to enhance the rate or extent of cellulose degradation by a cellulose-degrading enzyme mixture have been identified as belonging to GH Family 61. Recent investigations into the mechanisms of these polypeptides have shown that these are not glycoside hydrolases, but lytic polysaccharide monooxygenases (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). Accordingly, GH61 polypeptides have been reclassified within the CAZy system as Auxiliary Activity 9 (AA9) polypeptides. For the purposes herein, “GH61 polypeptides” and “AA9 polypeptides” are considered as equivalent classifications of polypeptides with cellulase enhancing activity.

In some embodiments of the present invention, the cellulose-degrading enzyme composition further comprises a GH61 or AA9 polypeptide. It is well known in the art that GH61 polypeptides exhibit cellulase-enhancing activity (see, for example, U.S. Pat. No. 7,608,869; U.S. Publication No. 2010/0306881A1; U.S. Pat. No. 7,741,466; U.S. Publication No. 2010/0143967A; WO2011/035072A2; U.S. Pat. No. 7,868,227; and WO2011/041397A1). In some embodiments, a GH61 or AA9 polypeptide exhibits from about 50% to about 100% amino acid sequence identity to Trichoderma reesei Cel61A (SEQ ID NO: 15) or M. thermophila Cel61P (SEQ ID NO: 18), from about 55% to about 100% amino acid sequence identity to M. thermophila Cel61A (SEQ ID NO: 19), or from about 65% to 100% amino acid sequence identity to M. thermophila Cel61F (SEQ ID NO: 17). For example, a GH61or AA9 polypeptide may be obtained or derived from any one of the organisms listed in Table 7 and demonstrate at least 50%, 60%, 70%, 80%, 85%, 90%, 95%, or 100% identity, or any % identity therebetween, to SEQ ID NO: 15 or SEQ ID NO: 18, at least 55%, 60%, 70%, 80%, 85%, 90%, 95%, or 100% identity, or any % identity therebetween, to SEQ ID NO: 19, or at least 65%, 70%, 80%, 85%, 90%, 95%, or 100% identity, or any % identity therebetween, to SEQ ID NO: 17. The GH61 or AA9 polypeptide may be functionally linked to a carbohydrate binding module (CBM) with a high affinity for crystalline cellulose, such as a Family 1 cellulose binding domain.

TABLE 7
Sequence Identity of GH61 or AA9 polypeptides Enzymes
to TrCel61A, MtCel61A, MtCel61F, and MtCel61P

| | | GenPept | |
|---|---|---|---|
| Organism | Protein | Accession | |
| | | | % Identity with |
| | | | amino acids of |
| | | | SEQ ID NO: 15 |
| | | | (TrCel61A) |
| Hypocrea rufa | Endoglucanase IV | ADJ57703.1 | 99.0 |
| Hypocrea orientalis | Endoglucanase IV | AFD50197.1 | 90.4 |
| Trichoderma sp. SSL | Endoglucanase IV | ACH92573.1 | 89.5 |
| Trichoderma | Type IV endoglucanase | ADB89217.1 | 89.3 |
| saturnisporum | | | |
| Trichoderam atroviride | Glycoside hydrolase family 61 | EHK46784.1 | 77.2 |
| IMI20040 | protein | | |
| Trichoderma virens | Glycoside hydrolase family 61 | EHK19374.1 | 74.7 |
| Gv29-8 | protein | | |
| Aspergillus terreus | Conserved hypothetical protein | XP_001213388.1 | 54.9 |
| NIH2624 | | | |
| Myceliophthora thermophila | Glycoside hydrolase family 61 | XP_003661787.1 | 54.2 |
| ATCC42464 | protein | | |
| Neurospora tetrasperma | Hypothetical protein | EGO61608.1 | 54.2 |
| FGSC 2508 | NEUT1DRAFT_77711 | | |
| Neosartorya fischeri | Endo-1,4-beta-glucanase, | XP_001259147.1 | 53.3 |
| NRRL 181 | putative | | |
| Aspergillu fumigatus | Endo-1,4-beta-glucanse | XP_748707.1 | 51.5 |
| Af293 | | | |
| Neurospora crassa | Endoglucanse IV precursor | XP_958254.1 | 51.2 |
| OR74A | | | |
| Magnaporthe oryzae 70-15 | Endoglucanse IV | EHA52001.1 | 50.1 |
| Thielavia terrestris NRRL 181 | Glycoside hydrolase family 61 | XP_003650513.1 | 49.7 |
| | protein | | |
| Aspergillus niger ATCC 1015 | Hypothetical protein | EHA27737.1 | 49.5 |
| | ASPNIDRAFT_53797 | | |
| | | | % Identity with |
| | | | SEQ ID NO: 19 |
| | | | (MtCel61A) |
| Chaetomium globosum | Hypothetical protein | XP_001225249.1 | 81.5 |
| CBS 148.15 | CHGG_07593 | | |
| Podospora anserina S | Hypothetical protein | XP_001911429.1 | 73.1 |
| mat+ | | | |
| Chaetomium | Hypothetical protein | EGS20667.1 | 67.5 |
| thermophilum var. | CTHT_0025030 | | |
| Thermphilym DSM 1495 | | | |
| Sordaria macrospora k- | Hypothetical protein | XO003346899.1 | 66.7 |
| hell | SMAC_05160 | | |
| Neurospora tetrasperma | Hypothetical protein | EGO61608.1 | 59.3 |
| FGSC 2508 | NEUTE1DRAFT_77711 | | |
| Neurospora crassa | Endoglucanase IV precursor | XP_958254.1 | 57.7 |
| OR74A | | | |
| Trichoderma | Type IV endoglucanase | ADB89217.1 | 56.2 |
| saturnisporum | | | |
| Hypocrea orientalis | Endoglucanase IV | AFD50197.1 | 55.9 |
| Trichoderma sp. SSL | Endoglucanase IV | ACH92573.1 | 55.9 |
| Trichoderma virens | Glycoside hydrolase family 61 | EHK19374.1 | 55.6 |
| Gv29-8 | protein | | |
| Trichoderma atroviride | Glycoside hydrolase family 61 | EHK46784.1 | 55.3 |
| IMI 206040 | protein | | |
| Trichoderma reesei | Endoglucanase 4 | O14405.1 | 55.0 |
| Magnaporthe oryzae 70-15 | Endoglucanase IV | EHA52001.1 | 54.7 |
| Hypocrea rufa | Endoglucanse IV | ADJ57703.1 | 54.1 |
| | | | Identity with |
| | | | SEQ ID NO 17: |
| | | | (MtCel61F) |
| Sordaria macrospora k- | Hypothetical proetin | XP_003345284.1 | 80.6 |
| hell | SMAC_04518 | | |
| Thielavia terrestris NRRL | Glycoside hydrolase family 61 | XP_003655380.1 | 78.1 |
| 8126 | protein | | |
| Chaetomium | Putative cellulose binding protein | EGS20384.1 | 75.7 |
| thermophilym var. | | | |
| thermophilum DSM 1495 | | | |
| Neurospora crassa | Hypothetical protein NCU08760 | XP_956109.1 | 75.4 |
| OR74A | | | |
| Neurospora tetrasperma | Hypothetical protein | EGO58503.1 | 75.4 |
| FGSC 2508 | NEUT1DRAFT_82948 | | |
| Podospora anserina S | Hypothetical protein | XP_001907702.1 | 70.3 |
| mat+ | | | |
| Magnaporthe oryzae 70-15 | Hypothetical protein | EHA50370.1 | 69.1 |
| | MGG_12733 | | |
| Neurospora tetrasperma | Hypothetical protein | EGO53245.1 | 65.7 |
| FGSC 2508 | NEUTE1DRAFT_92381 | | |
| Nerospora crassa OR74A | Hypothetical protein NCU01867 | XP_065498.1 | 64.5 |
| Pyrenophora teres f. teres | Hypothetical protein PTT_14450 | XP_003302575.1 | 64.3 |
| 0-1 | | | |
| Glomerella graminicola | Fungal cellulose binding domain- | EFQ34588.1 | 64.1 |
| M1.001 | containing protein | | |
| Thielavia terrestis NRRL | Glyoside hydrolase family 61 | XP_00365373.1 | 63.8 |
| 8126 | protein | | |
| Neurospora tetrasperma | Hypothetical protein | EGZ78102.1 | 63.5 |
| FGSC 2509 | NEUT2DRAFT_154588 | | |
| Sordaria macrospora k- | Hypothetical protein | XP_003349072.1 | 62.6 |
| hell | SMAC_06847 | | |
| | | | Identity with |
| | | | SEQ ID NO 18: |
| | | | (MtCel61P) |
| Chaetomium | Hypothetical protein | EGS19451.1 | 76.3 |
| thermophilum var. | CTHT_0049120 | | |
| thermophilum DSM 1495 | | | |
| Thielavia terrestris NRRL | Glycoside hydrolase family 61 | XP_003653998.1 | 75.4 |
| 8126 | protein | | |
| Podospora anserian S | Hypothetical protein | XP_001905623.1 | 73.6 |
| mat+ | | | |
| Neurospora ctetraperma | Hypothetical protein | EGO5569.1 | 70.5 |
| FGSC 2508 | NEUTE1DRAFT_67431 | | |
| Sordaria macrospora k- | Hypothetical proetin | XP_003351237.1 | 68.7 |
| hell | SMAC_03541 | | |
| Chaetomium globosum | Hypothetical proetin | XP_001219583.1 | 66.6 |
| CBS 148.51 | CHGG_00362 | | |
| Thielavia terrestris NRRL | Glycoside hyrolase family 61 | XP_00654493.1 | 57.4 |
| 8126 | protein | | |
| Arthrobotrys oligospora | Hypothetical protein | EGX50459.1 | 57.4 |
| ATCC 24927 | AOL_s00076g9 | | |
| Chaetomium globosum | Hypothetial protein | XP_001227732.1 | 57.4 |
| CBS 148.51 | CHGG_09805 | | |
| Myceliophthora | Glycosie hydrolase family 61 | XP_003665081.1 | 56.4 |
| thermophila ATCC4246 | protein | | |
| Pyrenophora teres f. teres | Hypothetical proetin PTT_18890 | XP_003305915 | 54.1 |
| 0-1 | | | |
| Podospora anserine S | Hypothetical protein | XP_001904958.1 | 53.5 |
| mat+ | | | |
| Glomerella graminicola | Glycosyl hydroase family 61 | EFQ25679.1 | 53.4 |
| M1.001 | | | |
| Magnaporthe oryzae 70-15 | Hypothetical protein MG_08066 | XP_362483.1 | 51.1 |

In some embodiments of the present invention, the cellulose-degrading enzyme composition further comprises a swollenin and/or a Cip protein. Cellulase enzyme mixtures comprising optimal ratios of swollenin, Cip1 and EG4 (a GH61 protein), have been shown to exhibit improved activity for the degradation of lignocellulosic substrates (U.S. Pat. No. 8,017,361).

“Swollenin” or “Swo1” is defined herein as any protein which exhibits the ability to swell or expand crystalline cellulose and comprises an amino acid sequence exhibiting at least 70%, 80%, 85%, 90%, 95% or 100% amino acid sequence identity to amino acids 92-475 (the expansin-like domain and its associated CBM) of the Trichoderma reesei Swollenin enzyme (SEQ ID NO: 14). Preferably, the Swollenin is functionally linked to a carbohydrate binding module (CBM) with a high affinity for crystalline cellulose, such as a Family 1 cellulose binding domain.

“Cip1” is defined herein as any protein, polypeptide or fragment thereof with about 40% to about 100% amino acid sequence identity, or more preferably about 56% to about 100% amino acid sequence identity, to amino acids 1-212 comprising the catalytic domain of the Trichoderma reesei Cip1 enzyme (SEQ ID NO: 13). Preferably, the Cip1 is functionally linked to a carbohydrate binding module (CBM) with a high affinity for crystalline cellulose, such as a Family 1 cellulose binding domain.

The cellulose-degrading enzyme composition of the present invention may further comprises one or more hemicellulase enzymes. Mixtures of cellulase and hemicellulases have been shown to be effective for the production of fermentable sugars from certain pretreated lignocellulosic substrates (Berlin et al., 2007, Biotechnology and Bioengineering, 97(2): 287-296). A hemicellulase, or hemicellulose degrading enzyme, is an enzyme capable of hydrolysing the glycosidic bonds in a hemicellulose polymer. Hemicellulases include, but are not limited to, xylanase (E. C. 3.2.1.8), beta-mannanase (E.C. 3.2.1.78), alpha-arabinofuranosidase (E.C. 3.2.1.55), beta-xylosidases (E.C. 3.2.1.37), and beta-mannosidase (E.C. 3.2.1.25). Hemicellulases typically comprise a catalytic domain of Glycoside Hydrolase Family 5, 8, 10, 11, 26, 43, 51, 54, 62 or 113.

The cellulose-degrading enzyme composition of the present invention may comprise enzymes that act on other biopolymers that are associated with cellulose in plant-derived biomass and feedstocks, such as lignin-degrading enzymes and esterases. Lignin-degrading enzymes are enzymes that oxidize and participate in the depolymerisation of lignin and include, for example, laccases (E.C. 1.10.3.2), lignin peroxidases (E.C. 1.11.1.14), manganese peroxidases (E.C. 1.11.1.13) and cellobiose dehydrogenases (E.C. 1.1.99.18). Examples of esterases which may be present in the cellulose-degrading enzyme composition include acetyl xylan esterases (E.C. 3.1.1.72) and ferulic acid esterases (E.C. 3.1.1.73). In addition, the cellulose-degrading enzyme composition may also include one or more additional enzyme activities such as pectinases, pectate lyases, galactanases, amylases, glucoamylases, glucuronidases, and galacturonidases.

### Genetically Modified Microbes Production the Cellulose-Degrading Enzyme Composition

The present invention also provides a genetically modified microbe for producing the cellulose-degrading enzyme composition. Such genetically modified microbe comprises an isolated polynucleotide encoding a GH16 polypeptide.

As used herein, an “isolated polynucleotide” is a polynucleotide that has been removed or separated from other polynucleotide material with which it is naturally associated and is suitable for use in a genetically modified microbe.

The isolated polynucleotide encoding a GH16 polypeptide, or “isolated GH16 polynucleotide”, may be derived from any one of a number of sources. For example, the isolated GH16 polynucleotide is preferably derived from fungal genera of the subdivision Ascomycotina or Basidiomycotina, including but limited to, Gloeophyllum, Geomyces, Coprinus, Leucosporidium, Phanerochaete, Schizophylum, Laccaria, Serpula, Piriformospora, Postia, Aspergillus, Rhodotorula, Lentinula, Cryptococcus, Myceliophthora, Thielavia, Botryotinia, Rhizopus, and taxonomic equivalents thereof. For example, the isolated GH16 polynucleotide may be derived from Gloeophyllum trabeum, Geomyces pannorum, Coprinus cinereus, Leucosporidium scottii, Phanerochaete chrysosporium, Schizophylum commune, Laccaria bicolor, Serpula lacrymans, Piriformospora indica, Postia placenta, Aspergillus fumigatus, Aspergillus nidulans, Rhodotorula glutinis, Lentinula edodes, Cryptococcus neoformans, and taxonomic equivalents thereof.

As used herein, in respect of polynucleotides, “derived from” refers to the isolation of a target polynucleotide sequence using one or more molecular biology techniques known to those of skill in the art including, but not limited to, reverse translation of a polypeptide or amino acid sequence, cloning, sub-cloning, amplification by PCR, in vitro synthesis, and the like. Furthermore, as is recognized by one of skill in the art, a polynucleotide sequence that is derived from a target polynucleotide sequence may be modified by one or more insertions, deletions and substitutions and still be considered to be “derived from” that target nucleotide sequence. Such one or more insertions, deletions and substitutions may result in increased or decreased expression or activity of the protein of interest encoded by the polynucleotide sequence and may be located within a promoter sequence, the 5′ or 3′ untranslated regions, or within the coding region for the protein of interest.

In some embodiments, the isolated GH16 polynucleotide is part of a genetic construct directing the expression and secretion of an isolated GH16 polypeptide from a genetically modified microbe. Such genetic construct typically contains regulatory sequences operably linked to the isolated GH16 polynucleotide that direct the expression and secretion of the encoded GH16 polypeptide, including: (i) a polynucleotide sequence encoding a secretion signal peptide from a secreted protein that may be endogenous or heterologous to the host microbe; and (ii) a constitutive or regulated promoter derived from a gene that is highly expressed in the host microbe under industrial fermentation conditions. In addition, a translational enhancer may be added to increase protein translation. These regulatory sequences may be derived from one or more genes, including, but not limited to, the gene encoding the GH16 polypeptide (provided that these regulatory sequences are functional in the host microbe). Moreover, multiple copies of the genetic construct(s) comprising an isolated GH16 polynucleotide may be introduced into the microbe, thereby increasing expression levels.

The genetic construct may comprise other polynucleotide sequences that allow it to recombine with sequences in the genome of the host microbe so that it integrates into the host genome. Alternatively, the genetic construct may not contain any polynucleotide sequences that direct sequence-specific recombination into the host genome. In such cases, the construct may integrate by random insertion through non-homologous end joining and recombination. Alternatively, the construct may remain in the host in non-integrated from, in which case it replicates independently from the host microbe's genome.

The genetic construct(s) may further comprise a selectable marker gene to enable isolation of a genetically modified microbe transformed with the construct as is commonly known to those of skill in the art. The selectable marker gene may confer resistance to an antibiotic or the ability to grow on medium lacking a specific nutrient to the host organism that otherwise could not grow under these conditions. The present invention is not limited by the choice of selectable marker gene, and one of skill in the art may readily determine an appropriate gene. For example, the selectable marker gene may confer resistance to hygromycin, phleomycin, kanamycin, geneticin, or G418, may complement a deficiency of the host microbe in one of the trp, arg, leu, pyr4, pyr, ura3, ura5, his, or ade genes, or may confer the ability to grow on acetamide as a sole nitrogen source.

The genetic construct may further comprise other polynucleotide sequences as is commonly known to those of skill in the art, for example, transcriptional terminators, polynucleotide sequences encoding peptide tags, synthetic sequences to link the various other polynucleotide sequences together, origins of replication, and the like. The practice of the present invention is not limited by the presence of any one or more of these other polynucleotide sequences.

The genetically modified microbe of the present invention results from the introduction of the above described isolated GH16 polynucleotide or genetic construct into a host microbe by any number of methods known by one skilled in the art, including but not limited to, treatment of cells with CaCl₂, electroporation, biolistic bombardment, PEG-mediated fusion of protoplasts (e.g. White et al., WO 2005/093072, which is incorporated herein by reference). After selecting the recombinant strains, such strains may be cultured in submerged liquid fermentations under conditions that enable the expression of an isolated GH16 polypeptide.

Suitable host microbes are yeasts and fungi of the phylum Ascomycota that produce one or more CBH and/or EG enzyme. The terms “fungus,” “fungi,” “fungal,” “Ascomycotina,” “Basidiomycotina” and related terms (e.g. “ascomycete” and “basidiomycete”) are meant to include those organisms defined as such in The Fungi: An Advanced Treatise (GC Ainsworth, FK Sparrow, AS Sussman, eds.; Academic Press 1973). Accordingly, it will be understood that, unless otherwise stated, the use of a particular genus and/or species designation in the present disclosure also refers to genera and species that are related by anamorphic or teleomorphic relationship, as well as genera and species that have been or may be reclassified into one of the claimed genera or species in the future. Examples of taxonomic equivalents can be found, for example, in Cannon, 1990, Mycopathologica 111:75-83; Moustafa et al., 1990, Persoonia 14:173-175; Stalpers, 1984, Stud. Mycol. 24; Upadhyay et al., 1984, Mycopathologia 87:71-80; Guarro et al., 1985, Mycotaxon 23: 419-427; Awao et al., 1983, Mycotaxon 16:436-440; von Klopotek, 1974, Arch. Microbiol. 98:365-369; and Long et al., 1994, ATCC Names of Industrial Fungi, ATCC, Rockville Md. Those skilled in the art will readily recognize the identity of appropriate equivalents.

Genera of yeasts useful as host microbes include Saccharomyces, Pichia, Hansenula, Kluyveromyces, Yarrowia, and Arxula. Genera of fungi useful as host include Trichoderma, Hypocrea, Aspergillus, Fusarium, Humicola, Neurospora, Myceliophthora, Thielavia, Sporotrichum, Chrysosporium, Penicillium, Coprinus, Leucosporidium, Geomyces, Gloeophyllum, Phanerochaete, Orpinomyces, Gibberella, Emericella, Acremonium, Chaetomium, and Magnaporthe. For example, the host microbe is an industrial strain of Trichoderma reesei, Myceliophthora thermophila, or Aspergillus nidulans.

The isolated GH16 polypeptide(s), one or more CBH and/or EG enzyme, and other enzymes and polypeptides of the cellulose-degrading enzyme composition may be homologous or endogenous to the host microbe(s) used to produce them or may be heterologous or exogenous to the host microbe(s). For purposes herein, a heterologous or exogenous enzyme or polypeptide is encoded by a gene derived from a species that is distinct from the species of the host microbe, as well as recognized anamorphs, teleomorphs or other taxonomic equivalents of the host microbe. An endogenous or homologous cellulase enzyme is encoded by a gene derived from the same species as the host microbe, as well as recognized anamorphs, teleomorphs or taxonomic equivalents of the host microbe. As is appreciated by one of skill in the art, the amino acid sequence of a homologous or heterologous enzyme or polypeptide may be naturally-occurring (i.e., as it is found in nature when produced by the source organism) or may contain one or more amino acid insertions, deletions or substitutions relative to the naturally-occurring amino acid sequence as a result of genetic manipulation, adaptation or classical mutagenesis causing changes in the polynucleotide sequence encoding said homologous or heterologous enzyme or polypeptide.

The isolated GH16 polypeptide and/or the one or more CBH and/or EG enzyme(s) of the cellulose-degrading enzyme composition, may be overexpressed from one or more host microbe(s). Overexpression refers to any state in which an enzyme or polypeptide is caused to be expressed at an elevated rate or level as compared to either (a) the endogenous expression rate or level of that same enzyme or polypeptide by the host microbe or (b) the expression rate or level of one or more other enzyme(s) or polypeptide(s) produced and secreted by the host microbe. As such, overexpression of the isolated GH16 polypeptide and/or the one or more CBH and/or EG enzymes(s) may result from increased expression of the isolated GH16 polypeptide and/or the one or more CBH and/or EG enzymes(s), as well as a decrease in expression of one or more other enzymes or polypeptides produced and secreted by the host microbe.

As is known by one of skill in the art, the increase or decrease in expression of a polypeptide or enzyme can be produced by any of various genetic engineering techniques. As used herein, the term genetic engineering technique refers to any of several well-known techniques for the direct manipulation of an organism's genes. For example, gene knockout (insertion of an inoperative DNA sequence, often replacing the endogenous operative sequence, into an organism's chromosome), gene knock-in (insertion of a protein-coding DNA sequence into an organism's chromosome), and gene knockdown (insertion of DNA sequences that encode antisense RNA or small interfering RNA, i.e., RNA interference (RNAi)) techniques are well known in the art. Methods for decreasing the expression of a polypeptide or enzyme also include partial or complete deletion of the encoding gene, and disruption or replacement of the promoter of the gene such that transcription of the gene is greatly reduced or even inhibited. As used herein, a gene deletion or deletion mutation is a mutation in which part of a sequence of the polynucleotide sequence making up the gene is missing. Thus, a deletion is a loss or replacement of genetic material resulting in a complete or partial disruption of the sequence of the DNA making up the gene.

Depending on the host microbe and the regulatory sequences directing their expression, the levels of the isolated GH16 polypeptide and/or the one or more CBH and/or EG enzyme in a given genetically modified microbe can be modulated by adjusting one or more parameters of the fermentation process used to produce the cellulose-degrading enzyme composition from the genetically modified microbe including, but not limited to, the carbon source, the temperature of the fermentation, or the pH of the fermentation. Yet another means for adjusting expression levels of the isolated GH16 polypeptide and/or the one or more CBH and/or EG enzyme in a given genetically modified microbe involves the modification of secretion pathways or modification of transcriptional and/or translational regulation systems and/or post-translational protein maturation machinery (e.g. transcription factors, protein chaperones). Changes in expression can also be achieved by mutagenesis and selection of strains with desired expression levels.

### Production of the Cellulose-Degrading Enzyme Composition

The isolated GH16 polypeptide(s), one or more CBH and/or EG enzyme, and other enzymes and polypeptides of the cellulose-degrading enzyme composition may be expressed and secreted from a single host microbe or from more than one host microbe. For example, the isolated GH16 polypeptide(s) may be produced by a host microbe that expresses one or more CBH or EG enzyme. The CBH and/or EG enzyme may be native or endogenous to the host microbe or may be produced from one or more isolated polynucleotide or genetic constructs encoding the one or more CBH and/or EG enzyme.

The cellulose-degrading enzyme composition of the present invention may be produced in a fermentation process in which one or more microbe(s) capable of expressing the isolated GH16 polypeptide(s), the one or more CBH enzyme(s) and/or EG enzyme(s), and other enzymes and polypeptides of the cellulose-degrading enzyme composition is grown in submerged liquid culture fermentation.

Submerged liquid fermentations of microorganisms, including industrial strains of Trichoderma, Myceliophthora, Aspergillus and taxonomically equivalent genera, are typically conducted as a batch, fed-batch or continuous process. In a batch process, all the necessary materials, with the exception of oxygen for aerobic processes, are placed in a reactor at the start of the operation and the fermentation is allowed to proceed until completion, at which point the product is harvested. A batch process may be carried out in a shake-flask or a bioreactor.

In a fed-batch process, the culture is fed continuously or sequentially with one or more media components without the removal of the culture fluid. In a continuous process, fresh medium is supplied and culture fluid is removed continuously at volumetrically equal rates to maintain the culture at a steady growth rate.

One of skill in the art is aware that fermentation medium comprises a carbon source, a nitrogen source, and other nutrients, vitamins and minerals which can be added to the fermentation media to improve growth and enzyme production of the host microbe. These other media components may be added prior to, simultaneously with, or after inoculation of the culture with the host microbe.

For the process for producing the isolated GH16 polypeptide(s), the one or more CBH enzyme(s) and/or EG enzyme(s), and other enzymes and polypeptides of the cellulose-degrading enzyme composition of the present invention, the carbon source may comprise a carbohydrate that will induce the expression of the isolated GH16 polypeptide(s), the one or more CBH enzyme(s) and/or EG enzyme(s), and other enzymes and polypeptides of the cellulose-degrading enzyme composition in the genetically modified microbe. For example, if the genetically modified microbe is a strain of a cellulolytic fungus such as Trichoderma or Myceliophthora, the carbon source may comprise one or more of cellulose, cellobiose, sophorose, xylan, xylose, xylobiose and related oligo- or poly-saccharides known to induce expression of cellulases and beta-glucosidase in such cellulolytic fungi. If the genetically modified microbe is a strain of Aspergillus in which the polynucleotides encoding the isolated GH16 polypeptide(s), the one or more CBH enzyme(s) and/or EG enzyme(s), and other enzymes and polypeptides of the cellulose-degrading enzyme composition are linked to regulatory sequences from amylase or glucoamylase genes, the carbon source may comprise one or more of starch, maltose, malto-oligosaccharides, and related di-, oligo- or poly-saccharides known to induce expression of starch-degrading enzymes in such fungi

In the case of batch fermentation, the carbon source may be added to the fermentation medium prior to or simultaneously with inoculation. In the cases of fed-batch or continuous operations, the carbon source may also be supplied continuously or intermittently during the fermentation process. For example, when the genetically modified microbe is a strain of Trichoderma or Myceliophthora, the carbon feed rate is between 0.2 and 4 g carbon/L of culture/h, or any amount therebetween.

The process for producing the isolated GH16 polypeptide(s), the one or more CBH enzyme(s) and/or EG enzyme(s), and other enzymes and polypeptides of the cellulose-degrading enzyme composition of the present invention may be carried at a temperature from about 20° C. to about 50° C., or any temperature therebetween, for example from about 25° C. to about 37° C., or any temperature therebetween, or from 20, 22, 25, 26, 27, 28, 29, 30, 32, 35, 37, 40, 45, 50° C. or any temperature therebetween.

The process for producing the isolated GH16 polypeptide(s), the one or more CBH enzyme(s) and/or EG enzyme(s), and other enzymes and polypeptides of the cellulose-degrading enzyme composition of the present invention may be carried out at a pH from about 3.0 to 8.5, or any pH therebetween, for example from about pH 3.5 to pH 7.0, or any pH therebetween, for example from about pH 3.0, 3.2, 3.4, 3.5, 3.7, 3.8, 4.0, 4.1, 4.2, 4.3, 4.4, 4.5, 4.6, 4.7, 4.8, 4.9, 5.0, 5.2, 5.4, 5.5, 5.7, 5.8, 6.0, 6.2, 6.5, 7.0, 7.5, 8.0, 8.5 or any pH therebetween.

Following fermentation, the fermentation broth(s) containing the isolated GH16 polypeptide(s), the one or more CBH enzyme(s) and/or EG enzyme(s), and other enzymes and polypeptides of the cellulose-degrading enzyme composition cellulose-degrading enzyme composition may be used directly, or the isolated GH16 polypeptide(s), the one or more CBH enzyme(s) and/or EG enzyme(s), and other enzymes and polypeptides of the cellulose-degrading enzyme composition cellulose-degrading enzyme composition may be separated from the fungal cells, for example by filtration or centrifugation. Low molecular weight solutes such as unconsumed components of the fermentation medium may be removed by ultrafiltration. The isolated GH16 polypeptide(s), the one or more CBH enzyme(s) and/or EG enzyme(s), and other enzymes and polypeptides of the cellulose-degrading enzyme composition cellulose-degrading enzyme composition may be concentrated, for example, by evaporation, precipitation, sedimentation or filtration. Chemicals such as glycerol, sucrose, sorbitol and the like may be added to stabilize the cellulose-degrading enzyme composition. Other chemicals, such as sodium benzoate or potassium sorbate, may be added to the cellulose-degrading enzyme composition to prevent growth of microbial contamination.

If the isolated GH16 polypeptide(s), the one or more CBH enzyme(s) and/or EG enzyme(s), and other enzymes and polypeptides of the cellulose-degrading enzyme composition are produced by more than one microbe, the microbes may be co-fermented to produce the composition. Alternatively, the broths from the fermentation of each microbe expressing one or more enzyme or polypeptide may be blended and used directly, or be blended and subjected to the purification, concentration and stabilization steps described above. Alternatively, the fermentation broths containing the individual enzymes and polypeptides may be added separately to a hydrolysis reaction containing a cellulosic substrate.

### Hydrolysis of Cellulosic Substrates

The cellulose-degrading enzyme composition of the present invention is useful for the production of fermentable sugars from a cellulosic substrate. By the term “fermentable sugar” it is meant any mono-, di-, or oligo-saccharide that can be converted by a microorganism into a useful product.

By the term “cellulosic substrate”, it is meant any substrate derived from plant biomass and comprising cellulose, including, but not limited to, pre-treated lignocellulosic feedstocks for the production of ethanol or other high value products, animal feeds, food products, forestry products, such as pulp, paper and wood chips, and textiles products. A cellulosic substrate may also be any one of a number of laboratory substrates known in the art, such as bacterial microcrystalline cellulose, Avicel, Sigmacel, acid-swollen cellulose, carboxymethyl cellulose, hydroxyethyl cellulose and azo-cellulose.

There are several assays known in the art for measuring the activity of a cellulose-degrading enzyme composition (or cellulase activity). It should be understood, however, that the practice of the present invention is not limited by the method used to assess cellulase activity. Methods to measure cellulase activity are published (e.g., Methods in Enzymology 160, Biomass Part A: Cellulose and Hemicellulose, Wood, W. A. and Kellogg, S. T., eds, Academic Press Inc. 1988; Ghose, T. K. (1987) Pure & Appl. Chem. 59(2):257-268) and include, for example, release of glucose or soluble oligo-saccharides from a cellulose substrate, release of a chromophore or fluorophore from a cellulose derivative, e.g., azo-CMC, or from a small, soluble substrate such as methylumbelliferyl-beta-D-cellobioside, para-nitrophenyl-beta-D-cellobioside,para-nitrophenyl-beta-D-lactoside and the like. For example, hydrolysis of cellulose can be monitored by measuring the enzyme-dependent release of reducing sugars, which are quantified in subsequent chemical or chemienzymatic assays known to one of skill in the art, including reaction with dinitrosalisylic acid (DNS). In addition, cellulose or colorimetric substrates (cellulose derivatives or soluble substrates) may be incorporated into agar-medium on which a host microbe expressing and secreting one or more cellulase enzymes is grown. In such an agar-plate assay, activity of the cellulase is detected as a colored or colorless halo around the individual microbial colony expressing and secreting an active cellulase.

Enzymatic hydrolysis of a cellulose substrate using the cellulose-degrading enzyme composition of the invention may be a batch process, a continuous process, or a combination thereof. The process may be agitated, unmixed, or a combination thereof.

The enzymatic hydrolysis is carried out at a pH and temperature that is at or near the optimum for the cellulose-degrading enzyme composition. For example, the enzymatic hydrolysis may be carried out at about 30° C. to about 75° C., or any temperature therebetween, for example a temperature of 30, 35, 40, 45, 50, 55, 60, 65, 70, 75° C., or any temperature therebetween, and a pH of about 3.5 to about 8.0, or any pH therebetween, for example a pH of 3.5, 4.0, 4.5, 5.0, 5.5, 6.0, 6.5, 7.0, 7.5, 8.0 or any pH therebetween.

The initial concentration of cellulose, prior to the start of enzymatic hydrolysis typically ranges from about 0.01% (w/w) to about 20% (w/w), or any amount therebetween, for example 0.01, 0.05, 0.1, 0.5, 1, 2, 4, 6, 8, 10, 12, 14, 15, 18, 20% (w/w) or any amount therebetween. Typical dosages for a cellulose-degrading enzyme composition range from about 0.001 to about 100 mg protein per gram cellulose, or any amount therebetween, for example 0.001, 0.01, 0.1, 1, 5, 10, 15, 20, 25, 30, 40, 50, 60, 70, 80, 90, 100 mg protein per gram cellulose or any amount therebetween.

Enzymatic hydrolysis of cellulose substrates are typically carried out for a time period of about 0.1 to about 200 hours, or any time therebetween, for example, the hydrolysis may be carried out for a period of 2 hours to 100 hours, or any time therebetween, or it may be carried out for 0.1, 0.5, 1, 2, 5, 7, 10, 12, 14, 15, 20, 25, 30, 35, 40, 45, 50, 55, 60, 65, 70, 75, 80, 85, 90, 95, 100, 120, 140, 160, 180, 200 hours or any time therebetween.

It should be appreciated that the reaction conditions are not meant to limit the invention in any manner and may be adjusted as desired by those of skill in the art.

The cellulose-degrading enzyme composition of the invention is useful for the enzymatic hydrolysis of a “pretreated lignocellulosic feedstock.” A pretreated lignocellulosic feedstock is a material of plant origin that, prior to pretreatment, contains at least 20% cellulose (dry wt), more preferably greater than about 30% cellulose, even more preferably greater than 40% cellulose, for example 20, 22, 24, 26, 28, 30, 32, 34, 36, 38, 40, 42, 44, 46, 48, 50, 55, 60, 65, 70, 75, 80, 85, 90% or any % therebetween, and at least 10% lignin (dry wt), more typically at least 12% (dry wt) and that has been subjected to physical and/or chemical processes to make the fiber more accessible and/or receptive to the actions of cellulolytic enzymes.

After pretreatment, the lignocellulosic feedstock may contain higher levels of cellulose. For example, if acid pretreatment is employed, the hemicellulose component is hydrolyzed, which increases the relative level of cellulose. In this case, the pretreated feedstock may contain greater than about 20% cellulose and greater than about 12% lignin. In one embodiment, the pretreated lignocellulosic feedstock contains greater than about 20% cellulose and greater than about 10% lignin.

Lignocellulosic feedstocks that may be used in the invention include, but are not limited to, agricultural residues such as corn stover, wheat straw, barley straw, rice straw, oat straw, canola straw, and soybean stover; fiber process residues such as corn fiber, sugar beet pulp, pulp mill fines and rejects, sugar cane bagasse or sugar cane leaves and tops; forestry residues such as aspen wood, other hardwoods, softwood, and sawdust; grasses such as switch grass, miscanthus, cord grass, and reed canary grass; or post-consumer waste paper products.

The lignocellulosic feedstock may be first subjected to size reduction by methods including, but not limited to, milling, grinding, agitation, shredding, compression/expansion, or other types of mechanical action. Size reduction by mechanical action can be performed by any type of equipment adapted for the purpose, for example, but not limited to, a hammer mill.

Non-limiting examples of pretreatment processes include chemical treatment of a lignocellulosic feedstock with sulfuric or sulfurous acid, or other acids; ammonia, lime, ammonium hydroxide, or other alkali; ethanol, butanol, or other organic solvents; or pressurized water (See U.S. Pat. Nos. 4,461,648, 5,916,780, 6,090,595, 6,043,392, 4,600,590, Weil et al., 1997, Applied Biochemistry and Biotechnology 68:21-40 and Ohgren, K., et al., 2005, Applied Biochemistry and Biotechnology 121-124:1055-1067; which are incorporated herein by reference).

The pretreatment may be carried out to hydrolyze the hemicellulose, or a portion thereof, that is present in the lignocellulosic feedstock to monomeric sugars, for example xylose, arabinose, mannose, galactose, or a combination thereof. Preferably, the pretreatment is carried out so that nearly complete hydrolysis of the hemicellulose and a small amount of conversion of cellulose to glucose occurs. During the pretreatment, typically an acid concentration in the aqueous slurry from about 0.02% (w/w) to about 2% (w/w), or any amount therebetween, is used for the treatment of the lignocellulosic feedstock. The acid may be, but is not limited to, hydrochloric acid, nitric acid, or sulfuric acid. For example, the acid used during pretreatment may be sulfuric acid.

One method of performing acid pretreatment of the feedstock is steam explosion using the process conditions set out in U.S. Pat. No. 4,461,648 (Foody, which is herein incorporated by reference). Another method of pretreating the feedstock slurry involves continuous pretreatment, meaning that the lignocellulosic feedstock is pumped through a reactor continuously. Continuous acid pretreatment is familiar to those skilled in the art; see, for example, U.S. Pat. No. 5,536,325 (Brink); WO 2006/128304 (Foody and Tolan); and U.S. Pat. No. 4,237,226 (Grethlein), which are each incorporated herein by reference. Additional techniques known in the art may be used as required such as the process disclosed in U.S. Pat. No. 4,556,430 (Converse et al.; which is incorporated herein by reference).

As noted above, the pretreatment may be conducted with alkali. In contrast to acid pretreatment, pretreatment with alkali does not hydrolyze the hemicellulose component of the feedstock, but rather the alkali reacts with acidic groups present on the hemicellulose to open up the surface of the substrate. The addition of alkali may also alter the crystal structure of the cellulose so that it is more amenable to hydrolysis. Examples of alkali that may be used in the pretreatment include ammonia, ammonium hydroxide, potassium hydroxide, and sodium hydroxide. The pretreatment is preferably not conducted with alkali that is insoluble in water, such as lime and magnesium hydroxide.

An example of a suitable alkali pretreatment is Ammonia Freeze Explosion, Ammonia Fiber Explosion or Ammonia Fiber Expansion (“AFEX” process). According to this process, the lignocellulosic feedstock is contacted with ammonia or ammonium hydroxide in a pressure vessel for a sufficient time to enable the ammonia or ammonium hydroxide to alter the crystal structure of the cellulose fibers. The pressure is then rapidly reduced, which allows the ammonia to flash or boil and explode the cellulose fiber structure. (See U.S. Pat. Nos. 5,171,592, 5,037,663, 4,600,590, 6,106,888, 4,356,196, 5,939,544, 6,176,176, 5,037,663 and 5,171,592, which are each incorporated herein by reference). The flashed ammonia may then be recovered according to known processes.

The pretreated lignocellulosic feedstock may be processed after pretreatment but prior to the enzymatic hydrolysis by any of several steps, such as dilution with water, washing with water, buffering, filtration, or centrifugation, or a combination of these processes, prior to enzymatic hydrolysis, as is familiar to those skilled in the art.

The pretreated lignocellulosic feedstock is next subjected to enzymatic hydrolysis. By the term “enzymatic hydrolysis”, it is meant a process by which cellulase enzymes act on cellulose to convert all or a portion thereof to soluble sugars. Soluble sugars are meant to include water-soluble hexose monomers and oligomers of up to six monomer units that are derived from the cellulose portion of the pretreated lignocellulosic feedstock. Examples of soluble sugars include, but are not limited to, glucose, cellobiose, cellodextrins, or mixtures thereof. The soluble sugars may be predominantly cellobiose and glucose. The soluble sugars may predominantly be glucose.

In the production of fermentable sugars by treatment of lignocellulosic feedstocks with the cellulose-degrading enzyme composition of the present invention, the enzymatic hydrolysis process preferably converts about 80% to about 100% of the cellulose to soluble sugars, or any range therebetween. More preferably, the enzymatic hydrolysis process converts about 90% to about 100% of the cellulose to fermentable sugars, or any range therebetween. In the most preferred embodiment, the enzymatic hydrolysis process converts about 95% to about 100% of the cellulose to fermentable sugars, or any range therebetween.

The enzymatic hydrolysis of pretreated lignocellulosic feedstocks is typically carried out in a hydrolysis reactor. The cellulose-degrading enzyme composition is added to the pretreated lignocellulosic feedstock prior to, during, or after the addition of the substrate to the hydrolysis reactor.

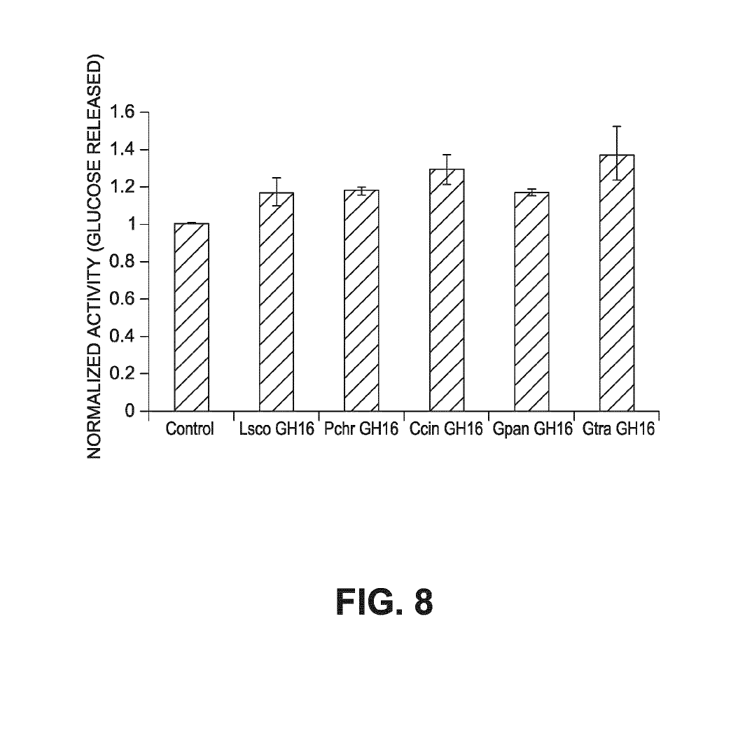

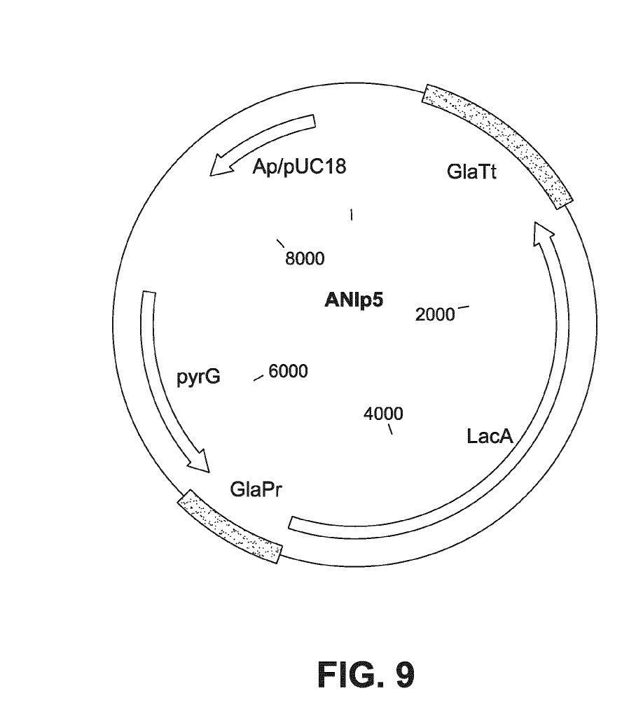

As shown in FIG. 8, cellulose-degrading enzyme compositions of the present invention that comprise an effective amount of an isolated GH16 polypeptide produce from about 10% to about 50% more glucose, a fermentable sugar, than a cellulose-degrading composition lacking an effective an isolated GH16 polypeptide.

The fermentable sugars produced by the enzymatic hydrolysis of cellulosic substrates may be converted by microbes to any number of fermentation products, including but not limited to ethanol, butanol, sugar alcohol, and lactic acid. For ethanol production, fermentation can be carried out by one or more than one microbe that is able to ferment the sugars to ethanol. For example, the fermentation may be carried out by recombinant Saccharomyces yeast that has been engineered to ferment glucose, mannose, galactose and xylose to ethanol, or glucose, mannose, galactose, xylose, and arabinose to ethanol. Recombinant yeasts that can ferment xylose to ethanol are described in U.S. Pat. No. 5,789,210 (which is herein incorporated by reference). The yeast produces a fermentation broth comprising ethanol in an aqueous solution. For lactic acid production, the fermentation can be carried out by a microbe that ferments the sugars to lactic acid.

The above description is not intended to limit the claimed invention in any manner. Furthermore, the discussed combination of features might not be absolutely necessary for the inventive solution.

### EXAMPLES

The present invention will be further illustrated in the following examples.

### Example 1

### Screening a Fungal Secretome for Activity on Pretreated Wheat Straw

1.1 Preparation of Fungal cDNA Libraries

Fungal cDNA libraries were prepared as previously described (Semova, et al., 2006, BMC Microbiology 6:7). Open reading frames (ORFs) encoding GH16 glycosyl hydrolases were PCR-amplified from full-length cDNAs identified by BLAST searches of the cDNA libraries. The selected GH16 ORFs were PCR amplified and cloned into expression vector ANIp5 (Storms et al. 2005, Plasmid 53:191-204). The forward and reverse primers used had at their 5′ ends five and six filler nucleotides followed by NheI and FseI restriction sites and lastly about 20 nucleotides of identity to the N-terminal and C-terminal portions of the ORF coding and noncoding strands, respectively. The amplified ORFs were ligated into the backbone of vector ANIp5 following digestion of the amplified ORFs and the vector by digestion with restriction endonucleases NheI and FseI.

1.2 Preparation of Aspergillus niger Spheroplasts

Spheroplasts of A. niger strain RS5775a (pyrG-6 cspA-1 ΔglaA::hisG ΔbglaA::hisG) or RS6525a (pyrG-6 cspA-1 ΔglaA::hisG dbglaA::hisG ΔargB ΔkusA Δ(aglU-prtT) amyA-prt7)::loxP dprtS1::loxP were generated using a modified version of the previously described method of Debets and Bos (1986, Fungal Genetics Newsletter 33, 24). Conidia from a stock plate or conidia suspension was streaked onto a complete media (CM) plate supplemented with uracil and uridine and incubated at 30° C. for 4 or 5 days. Conidia were harvested by washing the plate surface with Saline/Tween solution. A volume of 500 mL of CM media supplemented with uracil (110 mg/L) and uridine (240 mg/L) was inoculated with conidia at a final concentration of 2×10⁶ conidia/mL. The composition of the media is provided in Table 8 below. Cultures were incubated for 16 to 18 hours at 30° C. and 250 rpm. The germinated conidia were harvested by filtration through miracloth using a 9 cm Buchner funnel. Mycelial mass was washed with cold (4° C.) 0.6 M MgSO₄, transferred from the miracloth to a pre-weighed petri dish and the wet weight determined.

TABLE 8
CM + Uri + Ura medium

| Component | Concentration | |
|---|---|---|
| glucose | 10 | g/L |
| peptone | 2 | g/L |
| Yeast extract | 1 | g/L |
| Casamino acids | 1 | g/L |
| Uracil | 0.11 | g/L |
| Uridine | 0.24 | g/L |
| MgSO₄ | 0.01125M | |
| NaNO₃ (Sodium Nitrate) | 6 | g/L |
| KCL (Potassium Chloride) | 0.54 | g/L |
| KH₂PO₄ (Potassium Monobasic) | 0.815 | g/L |
| KH₂PO₄ (Potassium Monobasic) | 1.05 | g/L |
| ZnSO₄•7H₂O (Zinc Sulfate) | 22 | mg/L |
| H₃BO₃ (Boric Acid) | 11 | mg/L |
| MnCl₂•4H₂O (Manganous Chloride) | 5 | mg/L |
| FeSO₄•7H₂O (Ferrous Sulfate) | 5 | mg/L |
| CoCl₂•6H₂O (Cobaltous Chloride) | 1.6 | mg/L |
| CuSO₄•5H₂O (Cupric Sulfate) | 1.6 | mg/L |
| NH₄)₆Mo₂O₂₄•4H₂O (Ammonium molybdate) | 1.1 | mg/L |
| EDTA, tetrasodium salt | 65 | mg/L |
| EDTA, disodium salt | 7.7 | mg/L |
| Vitamin solution | 1 | ml per liter |
| (0.1 g/L pyridoxine-HCl, 0.15 g/L thiamine-HCl, | | |
| 0.75 g/L p-aminobenzoic acid, 2.5 g/L nicotinic | | |
| acid 2.5 g/l riboflavin, 20 g/L choline-HCl, | | |
| 0.025 g/L biotin) | | |

The weighed mycelial mass was transferred into a 100 mL flask and 5 mL of OM solution (1 M MgSO₄, 1.6 mM NaH₂PO₄, 8.4 mM Na₂HPO₄) per gram of mycelial mass was added followed by 125 mg of Glucanase (InterSpex Products Inc. San Mateo Calif. catalogue #0439-2) per gram of mycelial mass. The mycelia/glucanase suspension was incubated at 30° C. and 100 rpm for 1-3 hours until about 70-80% of the mycelia was converted into spheroplasts. The flask was then cooled in a 4° C. ice bath and the protoplast suspension transferred to a pre-cooled (4° C.) 50 mL Greiner tube. One volume of pre-cooled (4° C.) TB-solution (109.3 g/L sorbitol in 0.1 M Tris-HCl, pH 7.5) was carefully layered on top of the spheroplast suspension. After centrifugation at 3800 rpm for 30 min at 4° C., the spheroplasts were present as a turbid layer at the interface between TB-solution and OM-solution. The spheroplast layer was collected with a 10 mL transfer pipette, the harvested protoplasts transferred to a 50 mL Greiner tube and 45 mL of ice-cold S/C (1 M sorbitol, 50 mM CaCl₂) was added. After a 30 min centrifugation at 3000 rpm and 4° C., the fluid from the pelleted spheroplasts was decanted and the spheroplasts resuspended in 1 mL of ice-cold S/C. Resuspended spheroplasts were transferred into a 1.5 mL microcentrifuge tube and centrifuged for 5 min at 10,000 rpm and 4° C. The spheroplasts were resuspended in 1.5 mL S/C and the yield determined using a haemocytometer counting chamber. The spheroplasts were centrifuged for 5 min at 10,000 rpm and 4° C. and resuspended in ice-cold S/C at a final concentration of 1×10⁸ spheroplasts per mL. The protoplasts were kept on ice.

1.3 A. niger Transformation

Transformations were performed using a modified version of the previously described method of Wernars et al. (1987, Mol. Gen. Genet. 209, 71-77).

Spheroplasts were diluted to 1×10⁷/mL with ice-cold S/C. For each transformation, 40 μL, of spheroplasts suspension was combined with 4 μL of 0.4 M aurintricarboxylic acid, 5 μL of DNA (1-5 μg in TE), and 20 μL, of 20% PEG solution (20% w/v PEG 4000, 0.66 M sorbitol, 33 mM CaCl₂). The mixture was incubated for 10 minutes at room temperature (RT) followed by addition of 300 μL, of 60% (w/v) PEG solution. After careful mixing by pipetting, the mixture was incubated for 20 min at room temperature after which 1 mL of 1.2 M sorbitol was added. This mixture was centrifuged 5 min at 10,000 rpm and room temperature in a microcentrifuge and the pelleted spheroplasts resuspended in 200 μL of 1.2 M sorbitol. Prior to plating, the transformed spheroplasts were added to 10 mL of 48° C. molten medium (MM+KCl 0.6 M), quickly mixed by gentle vortexing and layered onto the surface of a MM+KCl 0.6 M agar plate (Table 9).

TABLE 9
MM + KCl 0.6M agar:

| Component | Concentration | |
|---|---|---|
| glucose | 10 | g/L |
| MgSO₄ | 0.01125M | |
| KCl (Potassium Chloride) | 44.7 | g/L |
| NaNO₃ (Sodium Nitrate) | 6 | g/L |
| KCL (Potassium Chloride) | 0.54 | g/L |
| KH₂PO₄ (Potassium Monobasic) | 0.815 | g/L |
| KH₂PO₄ (Potassium Monobasic) | 1.05 | g/L |
| ZnSO₄•7H₂O (Zinc Sulfate) | 22 | mg/L |
| H₃BO₃ (Boric Acid) | 11 | mg/L |
| MnCl₂•4H₂O (Manganous Chloride) | 5 | mg/L |
| FeSO₄•7H₂O (Ferrous Sulfate) | 5 | mg/L |
| CoCl₂•6H₂O (Cobaltous Chloride) | 1.6 | mg/L |
| CuSO₄•5H₂O (Cupric Sulfate) | 1.6 | mg/L |
| NH₄)₆Mo₂O₂₄•4H₂O (Ammonium molybdate) | 1.1 | mg/L |
| EDTA, tetrasodium salt | 65 | mg/L |
| EDTA, disodium salt | 7.7 | mg/L |
| Vitamin solution | 1 | mL per liter |
| (0.1 g/L pyridoxine-HCl, 0.15 g/L thiamine-HCl, | | |
| 0.75 g/L p-aminobenzoic acid, 2.5 g/L nicotinic | | |
| acid 2.5 g/l riboflavin, 20 g/L choline-HCl, | | |
| 0.025 g/L biotin) | | |
| Agar | 15 | g/L |

1.4 Production of GH16 Polypeptides from A. niger Transformants

A. niger transformants were grown in 100 mL of a minimal liquid medium (Kafer, 1977, Adv Genet 19:33-131) with 15% glucose as the carbon source for 5 days at 30° C. with shaking at 200 rpm. Culture supernatants were harvested by centrifugation at 3800×g for 20 minutes. Pretreated wheat straw was prepared using the methods described in U.S. Pat. No. 4,461,648. Following pretreatment, sodium benzoate was added at a concentration of 0.5% as a preservative. The pretreated material was then washed with six volumes of lukewarm (−35° C.) tap water using a Buchner funnel and filter paper.

1.5 Production of Fermentable Sugars from Pretreated Wheat Straw by Cellulose-Degrading Compositions Comprising GH16 Polypeptides

For each library polypeptide screened, an aliquot of culture filtrate (25 μL) from a host fungal strain expressing the polypeptide was added to a suspension of pretreated wheat straw (2% cellulose w/v) in 50 mM citrate buffer, pH 5.0, in a well of a 96-well microtitre plate. Culture filtrate from a strain transformed with an empty vector was used as the background control (i.e. no library polypeptide). A beta-glucosidase enriched cellulase mixture comprising cellobiohydrolases TrCel7A and TrCel6A, endoglucanases TrCel5A and TrCel7B, accessory proteins TrCel61A, Cip1, and swollenin, and low amounts of hemicellulases, secreted from T. reesei strain P59G (genetically modified to produce and secrete high levels of the TrCel3A beta-glucosidase using the methods of U.S. Pat. No. 6,015,703), was added to each well at a concentration of 0.05 mg/mL. The total volume in each well was 250 μL. The microplates were incubated for 48 hours at 50° C. with shaking (250 rpm; 1 inch radius) and then centrifuged for 3 min at 2800×g. An aliquot of supernatant from each well was removed and the amount of glucose released by the enzymatic hydrolysis of the cellulose by the cellulose-degrading enzyme mixtures was measured via the detection of glucose using a standard glucose oxidase/peroxidase coupled reaction assay (Trinder, 1969). Glucose released by the mixtures of library polypeptide with P59G cellulase was normalized to the control mixture of empty vector filtrate with P59G cellulase. Mixtures of the P59G cellulase and culture filtrates containing the Lsco GH16 (SEQ ID NO: 4), Pchr GH16 (SEQ ID NO: 6), Ccin GH16 (SEQ ID NO: 5), Gpan (SEQ ID NO: 7) or Gtra GH16 (SEQ ID NO: 3) polypeptides produced significantly more glucose from the pretreated wheat straw than a mixture of the P59G cellulase and a culture filtrate from the empty vector transformant (FIG. 8).

### Example 2

### Expression and Secretion of GH16 Polypeptides from Genetically Modified Microbes

2.1 Trichoderma reesei Strains

T. reesei strain P104F, a proprietary strain of logen Corporation derived from T. reesei strain BTR213, contains disruptions of the cel7a and cel6A genes generated by two consecutive steps of polyethylene glycol (PEG) mediated transformation of protoplasts and generation of uridine auxotrophs by plating on media containing 0.15% w/v 5-fluoroorotic acid (5-FOA) as previously described (U.S. Publication No. 2010/0221778). For deletion of the cel7a gene, a pyr4 auxotroph of strain BTR213 was transformed with p̂Clpyr4-TV (U.S. Publication No. 2010/0221778), a cel7a targeting vector containing the cel7a gene disrupted with a pyr4 selectable marker cassette. The isolated P54C strain possessing disruption of cel7a was then transformed with p̂C2pyr4-TV (U.S. Publication No. 2010/0221778), a cel6a targeting vector containing cel6a gene disrupted with pyr4 selectable marker cassette. The isolated P104F strain possessing disruption of both the cel7a and cel6a genes was plated on minimal media supplemented with 5 mM uridine and containing 0.15% w/v 5-FOA and uridine auxotroph P104Faux was isolated.

Trichoderma reesei strain P297J, a proprietary strain of Iogen Corporation, is a derivative of T. reesei strain BTR213 from which the genes encoding Cel7A, Cel6A and Cel7B have been deleted (U.S. Publication No. 2010/0221778). Strain BTR213 is a proprietary strain of Iogen Corporation derived from T. reesei strain RutC30 (ATCC 56765). The RutC30 strain was isolated as a high cellulase producing derivative of progenitor strain QM6A (Montenecourt and Eveleigh, 1979). Cellulase hyper-producing strains were generated from RutC30 by random mutation and/or selection. Strain M2C38 was isolated based on its ability to produce larger clearing zones than RutC30 on minimal media agar containing 1% acid swollen cellulose and 4 g L⁻¹ 2-deoxyglucose. Next, M2C38 was subjected to further random mutagenesis and strain BTR213 was isolated by selection on lactose media containing 0.2 μg/mL carbendazim. A uridine auxotroph of BTR213, BTR213aux, was obtained through selection of mutants spontaneously resistant to 0.15% w/v 5-FOA.

2.2 Genetic Constructs for Expression and Secretion of Isolated GH16 Polypeptides from a Fungal Host Microbe

Polynucleotides comprising the mature coding regions (i.e., the amino acid sequence starting after the putative secretion signal peptide to the stop codon) of the GH16 genes from Gloeophyllum trabeum (encoding Gtra GH16 of SEQ ID NO: 3), Phanerochaete chrysosporium (encoding Pchr GH16 of SEQ ID NO: 6), Leucosporidium scottii (encoding Lsco GH16 of SEQ ID NO: 4), Coprinus cinereus (encoding Ccin GH16 of SEQ ID NO: 5) and Geomyces pannorum (encoding Gpan GH16 of SEQ ID NO: 7) were synthesized by GenScript (Piscataway, N.J.). The GH16-coding sequences were codon-optimized for expression in T. reesei.

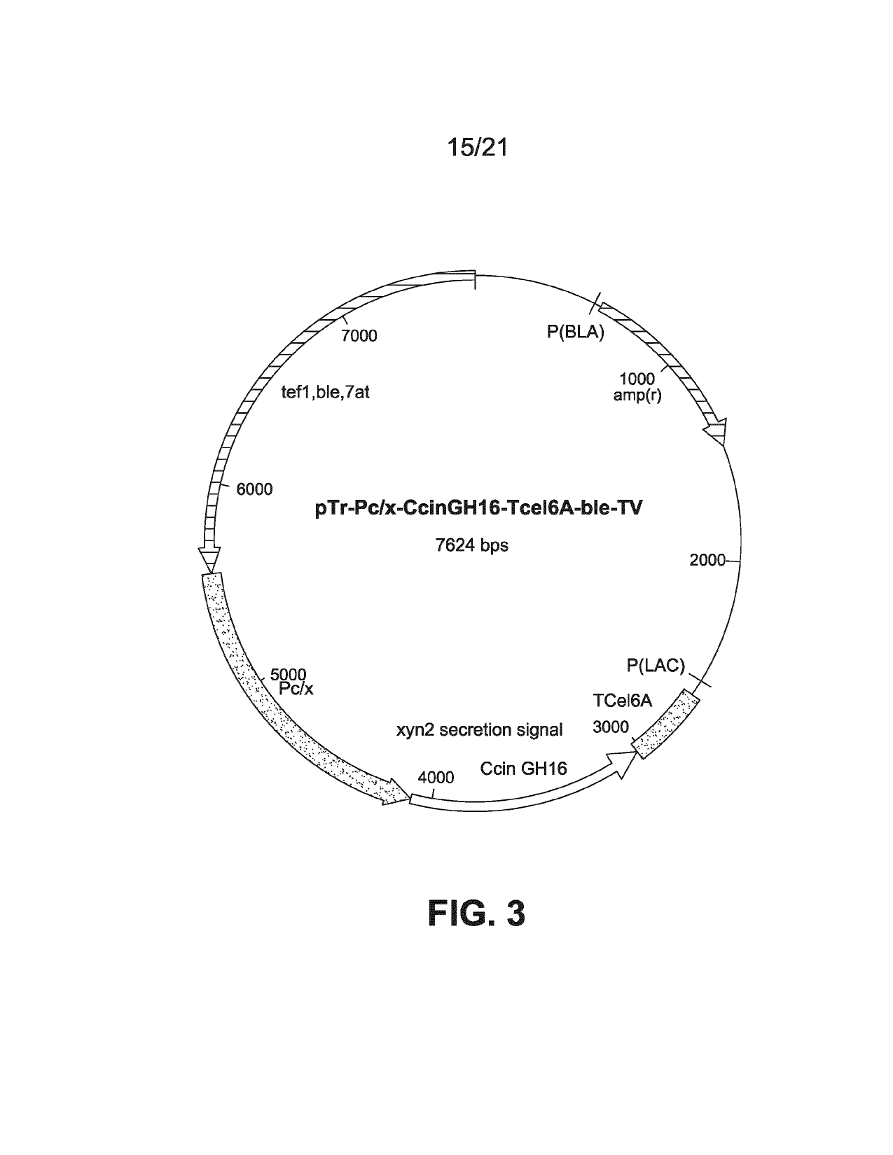

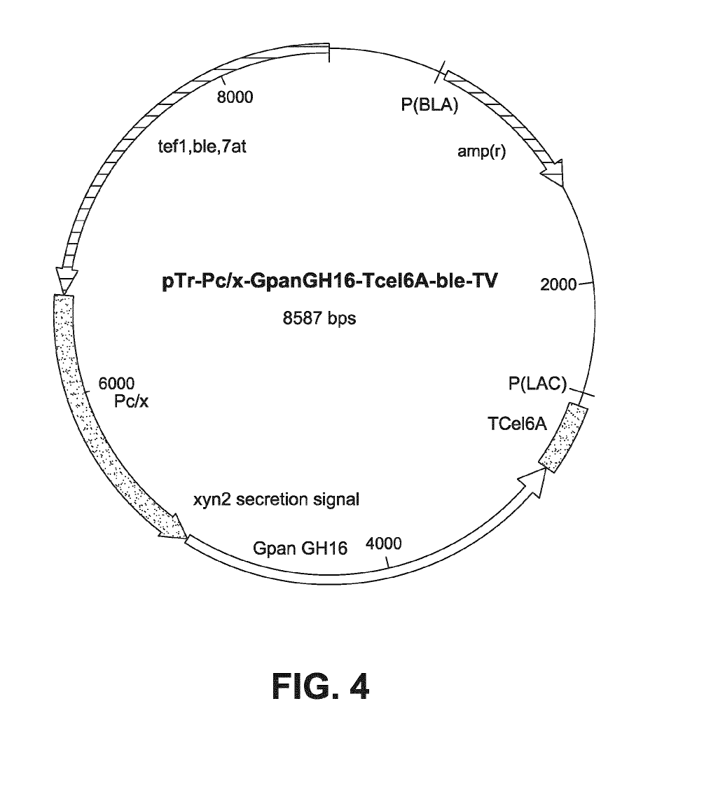

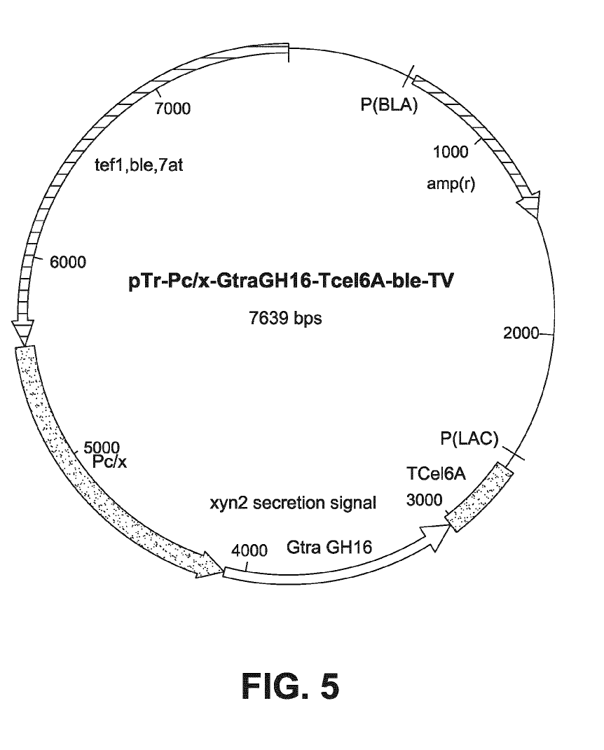

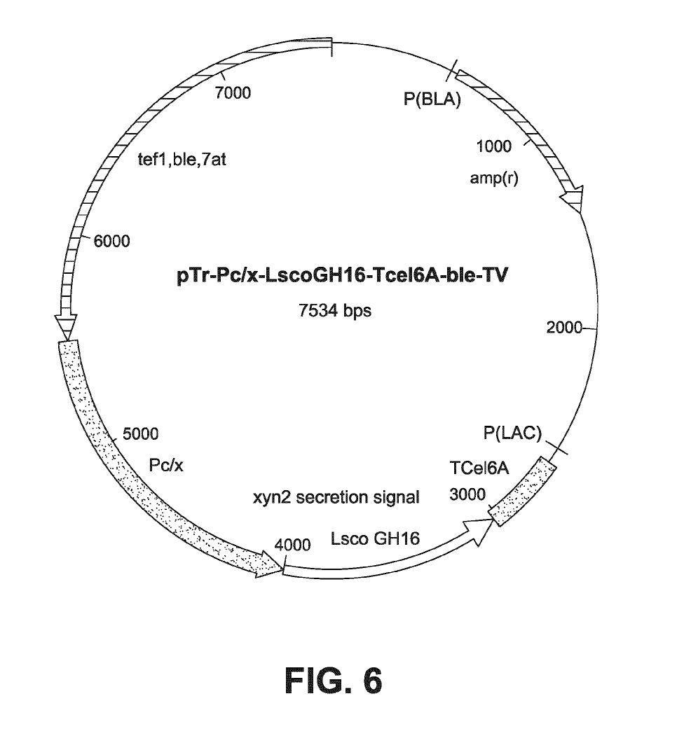

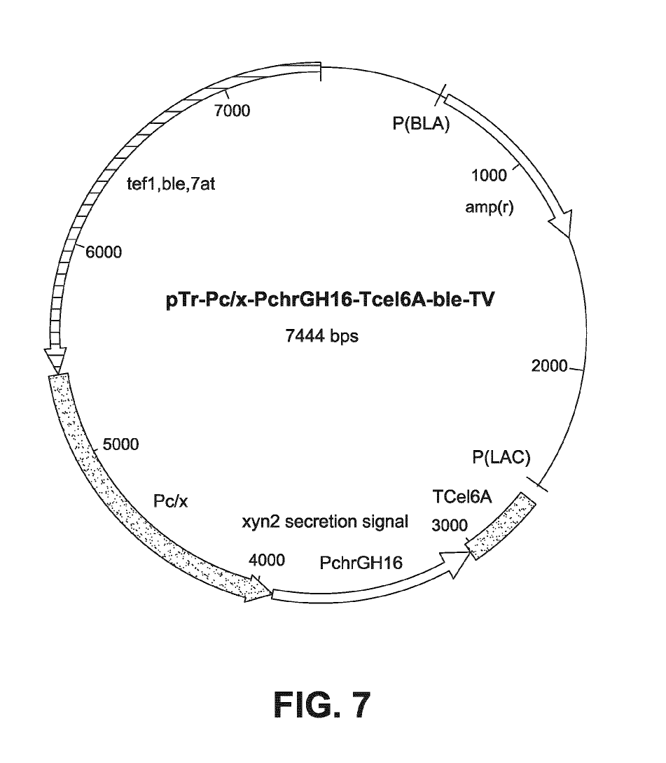

The T. reesei transformation vectors pTr-Pc/x-GtraGH16-Tcel7A-ble-TV (FIG. 5), pTr-Pc/x-LscoGH16-Tcel7A-ble-TV (FIG. 6), pTr-Pc/x-CcinGH16-Tcel7A-ble-TV (FIG. 3), pTr-Pc/x-PchrGH16-Tcel7A-ble-TV (FIG. 7) and pTr-Pc/x-GpanGH16-Tcel7A-ble-TV (FIG. 4) were constructed as follows. The synthetic polynucleotides comprising the coding regions of the GH16 genes were inserted into a Trichoderma transformation vector comprising a chimeric Trcel7A/xyn2 promoter (U.S. Pat. No. 6,015,703) in operative association with a the secretion signal coding sequence of the T. reesei xylanase 2 gene (Trxln2 ss) and the Trcel6A transcriptional terminator. The GH16 coding regions were inserted using a recombinase-based method to produce an in-frame fusion with the Trxln2 ss.

The transformation vectors also contain a Shble bleomycin resistance gene as a selectable marker. The Shble gene encodes the Streptoalloteichus hindustanus bleomycin resistance protein, ShBle, which confers resistance to bleomycin, zeocin and phleomycin. The transcription of the Shble gene is driven by the promoter (Ptefl) of the T. reesei tefl (transcription elongation factor 1) gene and terminated by a Trcel7a transcriptional terminator (Tcel7A).

Chemically-competent DH5α E. coli cells (Invitrogen cat No. 18265017) were transformed with each of the final transformation vectors shown in FIGS. 3 to 7. To generate DNA for the Trichoderma transformation, E. coli cells transformed with the plasmids were grown overnight in 5 mL liquid LB media supplemented with 75 μg/mL ampicillin with shaking at 37° C. Plasmid DNA for the transformations was isolated using the Wizard®Plus Miniprep Kit (Promega) as described in the manufacturer's protocol.

2.3 Transformation of T. reesei Host Microbes

T. reesei strain P297Jaux4 was transformed with the transformation vector pTr-Pc/x-GtraGH16-Tcel7A-ble-TV by biolistic gold particle bombardment using the PDS-1000/He system (BioRad; E.I. Dupont de Nemours and Company). Gold particles (median diameter of 0.6 μm, BioRad cat. No. 1652262) were used as micro-carriers. The HEPTA adapter was used with the following parameters: a rupture pressure of 1350 psi, a helium pressure of 1600 psi, and a target distance of 9 cm.

The spore suspension was prepared by washing T. reesei spores from PDAU (potato dextrose agar+5 mM uridine) plates incubated at 30° C. for 4-5 days with sterile water. Approximately 3.5×10⁸ spores were plated on 60 mm diameter plates containing PDAU+75 mg/mL phleomycin. After particle delivery, spores were washed from the transformation plate and moved to three 150 mm plates containing PDAU+75 mg/mL phleomycin (Invivogen, San Diego, Calif.). The plates were incubated at 30° C. for 5-8 days. All transformants were transferred to PDAU+75 mg/mL phleomycin media and incubated at 30° C.

T. reesei strain P104F was transformed in separate transformations with the transformation vectors pTr-Pc/x-GtraGH16-Tcel7A-ble-TV, pTr-Pc/x-LscoGH16-Tcel7A-ble-TV, pTr-Pc/x-CcinGH16-Tcel7A-ble-TV, and pTr-Pc/x-PchrGH16-Tcel7A-ble-TV by biolistic gold particle bombardment as described above. After particle delivery, spores were washed from the transformation plate and moved to three 150 mm plates containing PDA+75 mg/mL phleomycin (Invivogen). The plates were incubated at 30° C. for 5-8 days. All transformants were transferred to PDA+75 mg/mL phleomycin media and incubated at 30° C.

Transformants from the above transformations were cultured on PDA plates at 30° C. for 5-8 days or until sporulation. Spores were collected in Potato Dextrose Broth, 1 mL, and germinated at 30° C. for 38-42 h without shaking. Mycelia were centrifuged at 20,000×g for 5 min and the supernatant discarded. Solutions from the Promega Wizard Genomic DNA Purification Kit were used with a modified version of their published protocol 3.E. The mycelia pellets were transferred to a 1.5 mL micro-centrifuge tube containing glass beads and 600 μL of Nuclei Lysis Solution. The tubes were placed on a vortex mixer at top speed for 1 min and then incubated at 65° C. for 15 min. RNase Solution (3 μL) was mixed with the cell lysate and the whole mixture was incubated at 37° C. for 15 min. Once the tubes returned to room temperature, Protein Precipitation Solution was added (200 μL) and the tubes were mixed briefly. The proteins were precipitated by centrifugation at 16,000×g for 3 min. The supernatants were transferred to micro-centrifuge tubes containing 600 μL isopropanol. The genomic DNA samples were precipitated by centrifugation at 16,000×g for 1 min and the supernatants were removed. The DNA pellets were washed with 600 μL 70% ethanol and centrifugation at 16,000×g for 1 min. The supernatant was removed. The DNA pellets were air-dried at room temperature and then resuspended by adding 50 μL DNA Rehydration Solution and incubating at 65° C. for 1 h. The resultant genomic DNA was used as the templates (1 μL) in the subsequent PCR.

To confirm the integration of LscoGH16 gene (encoding the Leucosporidium scottii GH16 polypeptide of SEQ ID NO: 4), CcinGH16 gene (encoding the Coprinus cinerus GH16 polypeptide of SEQ ID NO: 5), and GpanGH16 gene (encoding the Geomyces pannorum GH16 polypeptide of SEQ ID NO: 7), primers AC382 (SEQ ID NO: 2) and AC250 (SEQ ID NO: 1) were used. The PCR was performed with Crimson Taq polymerase (New England Biolabs) according to the manufacturer's instructions with an annealing temperature of 55° C. Specific products of 1.2 kb (LscoGH16) and 1.3 kb (CcinGH16) were observed for the transformants but not in genomic DNA from the parent strain P104F. To confirm the integration of GtraGH16, gene primers AC382 (SEQ ID NO: 2) and SM054 (SEQ ID NO: 8) were used. The PCR was performed with Crimson Taq polymerase (New England Biolabs) according to the manufacturer's instructions with an annealing temperature of 56° C. The specific product of 970 bp was observed for the transformant but not in genomic DNA from the parent strain P104F or P2967Jaux4. T. reesei transformants expressing isolated GH16 polypeptides are listed in Table 10.

TABLE 10
Genetically Modified Microbes Expressing
Isolated GH16 Polypeptides

| | Host | | |
|---|---|---|---|
| | T. reesei | GH16 polypeptide | CBH or EG enzymes |
| Strain | strain | expressed | expressed |
| 4401A | P297Jaux | Gtra GH16 SEQ ID NO: 3 | TrCel5A |
| 4403S | P104F | Ccin GH16 SEQ ID NO: 5 | TrCel7B, TrCel5A |
| 4402P | P104F | Lsco GH16 SEQ ID NO: 4 | TrCel7B, TrCel5A |

### Example 3

### Production of Cellulose-Degrading Enzyme Compositions Comprising Isolated GH16 Polypeptides in Submerged Liquid Culture Fermentation

Trichoderma spores of transformants 4401A, 4402P, and 4403S were grown on PDA media, suspended in sterile water and transferred to 2 L, baffled Erlenmeyer flasks containing 750 mL of liquid Berkley media (pH 5.5) supplemented with 5.1 g/L of corn steep liquor powder and 10 g/L glucose (Table 11). Flasks were incubated at 28° C. for 3 days using an orbital agitator (Model G-52 New Brunswick Scientific Co.) running at 100 rpm.

TABLE 11
Berkley Media for Flasks

| Component | g/L |
|---|---|
| (NH₄)₂SO₄ | 10.4 |
| KH₂PO₄ | 2.0 |
| MgSO₄•7H₂O | 0.31 |
| CaCl₂•2H₂O | 0.53 |
| Dry Corn Steep Liquor | 5.1 |
| Glucose | 10 |
| Trace elements* | 1 mL/L |

*Trace elements solution contains 5 g/L FeSO₄•7H₂O, 1.6 g/L MnSO₄•H₂O and 1.4 g/L ZnSO₄•7H₂O.

The content of each inoculum flask was transferred to a 14 L pilot scale fermentation vessel (Model MF114 New Brunswick Scientific Co.) containing 10 L of Initial Pilot Media having a pH of 5.5 (Table 12). The vessel was run in batch mode until glucose in the media was depleted. At this point, the carbon source containing cellulase inducing carbohydrates was added on a continuous basis from a stock that was 35.5% w/v of solids dissolved in water. Peristaltic pumps were used to deliver the carbon source at a feed rate of 0.4 grams of carbon per liter culture per hour. Operational parameters during both the batch and fed-batch portions of the run were: mixing by impeller agitation at 500 rpm, air sparging at 8 standard liters per minute, and a temperature of 28° C. Culture pH was maintained at 4.0-4.5 during batch growth and pH 4.0 during cellulase production using an automated controller connected to an online pH probe and a pump enabling the addition of a 10% ammonium hydroxide solution. Periodically, 100 mL samples of broth were drawn for biomass and protein analysis.

TABLE 12
Initial Media for Fed-Batch Fermentations

| Component | g/L |
|---|---|
| (NH₄)₂SO₄ | 2.20 |
| KH₂PO₄ | 1.39 |
| MgSO₄•7H₂O | 0.70 |
| CaCl₂•2H₂O | 0.185 |
| Dry Corn Steep Liquor | 6.00 |
| Glucose | 13.00 |
| Trace elements* | 0.38 mL/L |

*Trace elements solution contains 5 g/L FeSO₄•7H₂O, 1.6 g/L MnSO₄•H₂O and 1.4 g/L ZnSO₄•7H₂O.

The biomass content of the culture broth was determined using aliquots of 5-10 mL of broth that had been weighed, vacuum filtered through glass microfiber filters, and oven dried at 100° C. for 4 to 24 hours. The concentration of biomass was determined according to the equation below.

$$Biomass ( g \text{/} L ) = \frac{dry filter paper and cake ( g ) - filter mass ( g )}{wet sample mass ( g )} \times broth density ( g \text{/} mL ) \times 1000 mL \text{/} L$$

The protein concentration of the culture filtrate was determined using the Bradford assay. Colour intensity changes in the Coomassie Brilliant Blue G-250 dye, that forms the basis of this assay, were quantified spectrophotometrically using absorbance measurements at 595 nm. The standard assay control used was a cellulase mixture of known composition and concentration. The final filtrates for enzyme analysis were collected after 162-170 hours.

### Example 4

### Purification of GH16 Polypeptides

Fungal cells from the culture filtrates from 14 L fed-batch fermentations of strains 4401A, 4402P and 4403S were removed from the fermentation broth by filtration across a glass microfiber filter containing a Harborlite filter bed.

A column of Phenyl Sepharose CL-4B (GE Healthcare, catalogue #17-0810-01) was packed in a 16/40 XK column (catalogue #28-9889-38) from GE Healthcare. The packed resin volume was about 65 mL. The column was equilibrated in 10 mM sodium phosphate, pH 7.5 and 1.5 M ammonium sulfate (Buffer 1). The cellulase mixtures were adjusted to Buffer 1 salt and pH conditions and applied to the column at 3 mL/min After sample application, unbound proteins in the load were washed through the column with five bed volumes of Buffer 1. Bound proteins were eluted using a six column volume decreasing linear 1.5 to 0 M ammonium sulfate gradient in 10 mM sodium phosphate, pH 7.5 (Buffer 2). The flow rate during the elution gradient was 3 mL/min and 4 mL fractions were collected.

Fractions were analyzed for activity on CM-curdlan (Megazyme, catalogue #P-CMCUR). The stock substrate was prepared by gradually dissolving 200 mg of CM-curdlan in 20 mL of warm 100 mM sodium citrate, pH 5.0 while stiffing. A volume of 50 μL of selected column fractions was incubated with 50 μL of stock reagent for 16 h at 50° C. At the end of the incubation, 80 μL of DNS reagent (Table 13) was added to each well and incubated at 100° C. for 10 min before cooling to room temperature. Absorbance of each sample at 540 nm was measured in a 96 well microtitre plate. Reducing sugar concentrations were calculated using a glucose standard curve.

TABLE 13
DNS reagent

| Component | g/L |
|---|---|
| 3,5-Dinitosalicylic acid (Acros) | 10 |
| Sodium hydroxide (Fisher) | 10 |
| Phenol (Sigma) | 2 |
| Sodium metabisulfate (Fisher) | 0.5 |

Fractions enriched in curdlan activity were pooled and the GH16 polypeptides further isolated by anion exchange chromatography. The load was adjusted to 20 mM sodium phosphate, pH 7.0 (Buffer 3) and applied to a 65 mL column of DEAE Sepharose FF (GE Healthcare, catalogue #17-0709-60) pre-equilibrated in Buffer 3. Unbound proteins were washed through the column with five column volumes of Buffer 3. Bound proteins were eluted with a 0-300 mL NaCl gradient in 20 mM sodium phosphate, pH 7.0 (Buffer 4). The flow rate in all steps was 3 mL/min and 15 mL fractions were collected during the elution. Fractions containing the GH16 enzymes in each run were identified using the curdlan activity assay described above.

After purification, the GH16 polypeptides were concentrated and buffer exchanged into 50 mM sodium citrate, pH 5.0 using a stirred ultrafiltration cell (Amicon) and a 10 kDa NMWL polyethersulfone membrane. Protein concentrations were measured using a BCA assay kit from Sigma (catalogue #BCA-1).

1-21. (canceled)

22. A cellulose-degrading enzyme composition comprising, one or more cellobiohydrolase or endoglucanase enzymes, and an effective amount of at least one GH16 polypeptide, where the presence of the at least one GH16 polypeptide in the enzyme composition increases the rate or extent of degradation of a cellulosic substrate compared to an otherwise equivalent cellulose-degrading enzyme composition comprising the same one or more cellobiohydrolase or endoglucanase enzyme but lacking the at least one GH16 polypeptide, and wherein the at least one GH16 polypeptide comprises an amino acid sequence exhibiting at least 90% identity to SEQ ID NO: 3, SEQ ID NO: 4, SEQ ID NO: 5, or SEQ ID NO: 7.

23. The cellulose-degrading enzyme composition of claim 22, wherein the source of the one or more GH16 polypeptides is one or more of Gloeophyllum trabeum, Geomyces pannorum, Coprinus cinereus, Leucosporidium scottii, Schizophylum commune, Laccaria bicolor, Serpula lacrymans, Piriformospora indica, Postia placenta, Aspergillus fumigatus, Aspergillus nidulans, Rhodotorula glutinis, Lentiula edodes, Cryptococcus neoformans, and taxonomic equivalents thereof.

24. The cellulose-degrading enzyme composition of claim 23, wherein the at least one GH16 polypeptide exhibits at least 95% identity to SEQ ID NO: 3, SEQ ID NO: 4, SEQ ID NO: 5, or SEQ ID NO: 7.

25. The cellulose-degrading enzyme composition of claim 23, wherein the at least one GH16 polypeptide comprises SEQ ID NO: 3, 4, 5, or 7.

26. The cellulose-degrading enzyme composition for claim 22 comprising, one or more cellobiohydrolase enzymes and one or more endoglucanase enzymes.

27. The cellulose-degrading composition of claim 22, wherein the one or more cellobiohydrolase or endoglucanase enzymes are native or variant enzymes of a fungal cell from the genus Trichoderma or Myceliophthora.

28. The cellulose-degrading composition of claim 27, wherein the fungal cell is Trichoderma reesei or Myceliophthora thermophile.

29. The cellulose-degrading enzyme composition of claim 22, wherein
the one or more cellobiohydrolase enzyme is a GH7 or GH6 cellobiohydrolase; and
the one or more endoglucanase enzyme is a GH7 or GH5 endoglucanase.

30. The cellulose-degrading enzyme composition of claim 29, wherein
the GH7 cellobiohydrolase comprises an amino acid sequence exhibiting from about 60 to 100% identity to amino acids 1 to 436 of SEQ ID NO: 9 or to amino acids 1 to 438 of SEQ ID NO: 20;
the GH6 cellobiohydrolase comprises an amino acid sequence exhibiting from about 45 to 100% identity to amino acids 83-447 of SEQ ID NO: 10 or to amino acids 118-432 of SEQ ID NO: 23;
the GH7 endoglucanase comprises an amino acid sequence exhibiting from about 48% to 100% identity to amino acids 1 to 374 of SEQ ID NO: 16 or from about 65% to 100% identity to amino acids 30-390 of SEQ ID NO: 24; and
the GH5 endoglucanase comprises an amino acid sequence exhibiting from about 40% to 100% identity to amino acids 202 to 222 of SEQ ID NO: 11 or from about 65% to 100% identity to amino acids 77 to 297 of SEQ ID NO: 22.

31. The cellulose-degrading enzyme composition of claim 22, further comprising a beta-glucosidase enzyme.

32. The cellulose-degrading enzyme composition of claim 22, further comprising a GH61 polypeptide.

33. The cellulose-degrading enzyme composition of claim 22, further comprising one or more hemicellulase, one or more cellulase-enhancing protein, one or more lignin-degrading enzymes, or one or more esterases.

34. The cellulose-degrading enzyme composition of claim 22, wherein the one or more cellobiohydrolase or endoglucanase enzyme and the GH16 polypeptide are produced by a single genetically modified microbe.

35. The cellulose-degrading enzyme composition of claim 22, wherein the GH16 polypeptide is produced by a genetically modified microbe and then blended with the one or more cellobiohydrolase or endoglucanase enzyme produced by one or more other microbe.

36. A method for producing fermentable sugars comprising treating a cellulosic substrate with a cellulose-degrading enzyme composition of claim 22.

37. The method of claim 36, wherein the cellulosic substrate is a pretreated lignocellulose feedstock.

38. The method of claim 37, wherein the pretreated lignocellulose feedstock is selected from the group consisting of corn stover, wheat straw, barley straw, rice straw, oat straw, canola straw, soybean stover, corn fiber, sugar beet pulp, pulp mill fines and rejects, sugar cane bagasse, sugar cane leaves, sugar cane tops, hardwood, softwood, sawdust, switch grass, miscanthus, cord grass, and reed canary grass.

39. A method for producing a fermentable product comprising treating a cellulosic substrate with a cellulose-degrading enzyme composition of claim 22 to produce fermentable sugars; and fermenting the fermentable sugars.

40. A genetically modified microbe for producing a cellulose-degrading composition comprising, at least one polynucleotide encoding a cellobiohydrolase enzyme or an endoglucanase enzyme, and an polynucleotide encoding a GH16 polypeptide, wherein the GH16 polypeptide comprises an amino acid sequence exhibiting at least 90% identity to SEQ ID NO: 3, SEQ ID NO: 4, SEQ ID NO: 5, or SEQ ID NO: 7.

41. The genetically modified microbe of claim 40, wherein the GH16 polypeptide comprises an amino acid sequence exhibiting from about 95 to 100% identity to SEQ ID NO: 3, 4, 5, or 7.


SEQUENCE LISTING (102 sequences)

SEQ ID NO: 1 - DNA, 21 bp, Artificial Sequence
  Primer sequence
ggaaccacaccatcgcacatc

SEQ ID NO: 2 - DNA, 22 bp, Artificial Sequence
  PCR primer
gcacctgaacagtcatacaacc

SEQ ID NO: 3 - protein, 360 aa, Gloeophyllum trabeum
YTLVHEFSGQTFFNGWDFFDGYDPTTYGDTTYLNQAEAQAAHLAYVDSNSGHAFIRVDNTTNVPDQQKRNTIEIFTHEFYPVGSVFILDAVHIPWGCSVWPSFWTRGENWPYGGEIDIIEYANLMGFNQMALHTSAGCTHTTPQSQVGQTLEPNCNATSGAGCTVAEKKPNSYGPGFASAGGGVWATQFDVSGIYIWFWSRPDVPPSLSLANTTIDPASWGPPSASYPASSCDIGSHFAAQQLVMDIQLCGAFGNPTYNNTCGPGSCYDLSVRGPGSPTYDNAYFEISYVRVFGTGNATSSNSSTSTTGTATATKSGSPTATHSAPASGGDNTSGSAPGLSQPFALFSVVGLLLLGSAVL

SEQ ID NO: 4 - protein, 325 aa, Leucosporidium scottii
DYSLEVAYQGESFFNGWDYWGNRDNLTNGAVYYVAKDESSDFTYTNSAGNAIIKVDNETTLASGSNALRNSVRITTQAAYDIGSLIVMDALHVPYGCATWPAFWMKAKEWPSGGEVDMFETVNMQKNAVALHSTIGCYAANSTATDSASGTMSFNDCNYQVAANHGCTFESPTNASYGASFAEVGGGIYATELASDAISVWFFPRADIPADLRAVNGTPDPKSWGLPVAYYPSSSCNINQYFAPQQITINIALCGSWAGEPGVFSPACGTGLCADYVLNPAHFDEAYFEIASVRVYSGGLNTRSSSGGVAASGVIGAIGGAGSAT

SEQ ID NO: 5 - protein, 355 aa, Coprinus cinereus
SRNRSEPLERRNQYLDRNRDGTPFVWLLEDDYKGHDFFDHFEFFNWTDPTNGMVRYVSREEAFARRLAYVQDDGIVVMKADDTSHLPRGEFRSSVRINTIKRYTTGLFILDLNTAPWGCGVWPAWWSTGDNWPVSGEIDIIEGVHDNEHNQIAWHTEPGCVLDTEESFTGNVSIKSGGPAVECNAHINQNAGCSITEWSRASYGPYFDEQGGGVFAMKWDENGIAVWSFYRAAIPKDITEGNPNPRNWGDPSALLGPGKCNIMEYFRNHTVILNITFCGDWAGNSYATSGCPGTCPDRLMDPANFVNATWSINSMKVYRKQPIYAEVVDPNKSAASRNVLGSLALVPLVGAALMN

SEQ ID NO: 6 - protein, 295 aa, Phanerochaete chrysosporium
GSYTLIDNYVGSTFLSAFVHEAIADPTHGRVNYVNQATAVAKNLTFASGNTLILRADDTTVLSPSGPGRNSVRIRSVKAYTTHVAIIDVRHMPQGCGTWPAFWETDGSNWPNGGEVDIIEGVNDQSPNAMTLHTGANCNMPASRAETGTPTGLNCDVNTDGNTGCGVQAPTANSYGPALNAIGGGWYAMERTNNFIKVWFFPRNGNTPSDLKNGASSINTDNWGTPTAFFPNTNCDIGSHFDQNNIIINLTFCGDWAGAVYGNSGCPSTCVDYVNNNPSAFKNAYWDIAAVRVYE

SEQ ID NO: 7 - protein, 676 aa, Geomyces pannorum
AVAAVPTEYTVADVYQGQTFFDQFNFNSDVDPTHGFVDFKAHDAAKSQGLISVNTLGQAYLGVDSTTVLTNLNQRGRASVRVESKKLYNGGLFIADIAHMPSSVCGVWPAFWTSGQQNWPNDGEIDIIENISETQQNAVTLHTGTEDCTISKSAQGGTLVTSHCSNYYADHVTQWENQGCSVQSTDSSNNYGDSFNTVGGGIYALEWTGTTIKIWNFPRLSADGIDALSAHPDPTKWRKATITTVGGSCDVTKLFKNHNLIIDTTFCGDYAGQDVFWQATTCYKSNPTKYASCASYVAANPTKYKDAYWLINSVKVYQSGPKSVSSSTQAPTSTKASTSTKASTSTKASTSTKASTSTKASTSTKASTSTKASTSTKAATSTDAATSTDSATSTDSATSTESATSTDAATSTESATSTDAATSTDAATSTDAATSTDAATSTDYVHPTTPVGTLSSKPYSNSSTTAPTSYPTLTSTVITTSVYTVTSCAPTVTNCPVGHVTTDIITSLTTWCPGNPTYTPVPTTTPGSDEYTTSTVYATNIVTVTKCPQTVTNCPASSTVVVTSVYPVSTTVCPVGPLPTYSGLPSVIPPYGNGNGTSPAGPTGPAGPGGPANPTKSSAPAGPGGPANPTKSSAPVQPSKPVIPVQPSSPIFGSSGDRIGASLTLLMGVAGVALLL

SEQ ID NO: 8 - DNA, 20 bp, Artificial Sequence
  Primer
cgcaggtgttgttgtaagtg

SEQ ID NO: 9 - protein, 497 aa, Trichoderma reesei [sequence omitted]

SEQ ID NO: 10 - protein, 447 aa, Trichoderma ressei [sequence omitted]

SEQ ID NO: 11 - protein, 397 aa, Trichoderma ressei
QQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNDDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 12 - protein, 714 aa, Trichoderma reesei
AVVPPAGTPWGTAYDKAKAALAKLNLQDKVGIVSGVGWNGGPCVGNTSPASKISYPSLCLQDGPLGVRYSTGSTAFTPGVQAASTWDVNLIRERGQFIGEEVKASGIHVILGPVAGPLGKTPQGGRNWEGFGVDPYLTGIAMGQTINGIQSVGVQATAKHYILNEQELNRETISSNPDDRTLHELYTWPFADAVQANVASVMCSYNKVNTTWACEDQYTLQTVLKDQLGFPGYVMTDWNAQHTTVQSANSGLDMSMPGTDFNGNNRLWGPALTNAVNSNQVPTSRVDDMVTRILAAWYLTGQDQAGYPSFNISRNVQGNHKTNVRAIARDGIVLLKNDANILPLKKPASIAVVGSAAIIGNHARNSPSCNDKGCDDGALGMGWGSGAVNYPYFVAPYDAINTRASSQGTQVTLSNTDNTSSGASAARGKDVAIVFITADSGEGYITVEGNAGDRNNLDPWHNGNALVQAVAGANSNVIVVVHSVGAIILEQILALPQVKAVVWAGLPSQESGNALVDVLWGDVSPSGKLVYTIAKSPNDYNTRIVSGGSDSFSEGLFIDYKHFDDANITPRYEFGYGLSYTKFNYSRLSVLSTAKSGPATGAVVPGGPSDLFQNVATVTVDIANSGQVTGAEVAQLYITYPSSAPRTPPKQLRGFAKLNLTPGQSGTATFNIRRRDLSYWDTASQKWVVPSGSFGISVGASSRDIRLTSTLSVA

SEQ ID NO: 13 - protein, 297 aa, Trichoderma reesei
QISDDFESGWDQTKWPISAPDCNQGGTVSLDTTVAHSGSNSMKVVGGPNGYCGHIFFGTTQVPTGDVYVRAWIRLQTALGSNHVTFIIMPDTAQGGKHLRIGGQSQVLDYNRESDDATLPDLSPNGIASTVTLPTGAFQCFEYHLGTDGTIETWLNGSLIPGMTVGPGVDNPNDAGWTRASYIPEITGVNFGWEAYSGDVNTVWFDDISIASTRVGCGPGSPGGPGSSTTGRSSTSGPTSTSRPSTTIPPPTSRTTTATGPTQTHYGQCGGIGYSGPTVCASGTTCQVLNPYYSQCL

SEQ ID NO: 14 - protein, 469 aa, Trichoderma reesei
LFGQCGGIGWSGTTCCVAGAQCSFVNDWYSQCLASTGGNPPNGTTSSSLVSRTSSASSSVGSSSPGGNSPTGSASTYTTTDTATVAPHSQSPYPSIAASSCGSWTLVDNVCCPSYCANDDTSESCSGCGTCTTPPSADCKSGTMYPEVHHVSSNESWHYSRSTHFGLTSGGACGFGLYGLCTKGSVTASWTDPMLGATCDAFCTAYPLLCKDPTGTTLRGNFAAPNGDYYTQFWSSLPGALDNYLSCGECIELIQTKPDGTDYAVGEAGYTDPITLEIVDSCPCSANSKWCCGPGADHCGEIDFKYGCPLPADSIHLDLSDIAMGRLQGNGSLTNGVIPTRYRRVQCPKVGNAYIWLRNGGGPYYFALTAVNTNGPGSVTKIEIKGADTDNWVALVHDPNYTSSRPQERYGSWVIPQGSGPFNLPVGIRLTSPTGEQIVNEQAIKTFTPPATGDPNFYYIDIGVQFSQN

SEQ ID NO: 15 - protein, 323 aa, Trichoderma reesei
HGHINDIVINGVWYQAYDPTTFPYESNPPIVVGWTAADLDNGFVSPDAYQNPDIICHKNATNAKGHASVKAGDTILFQWVPVPWPHPGPIVDYLANCNGDCETVDKTTLEFFKIDGVGLLSGGDPGTWASDVLISNNNTWVVKIPDNLAPGNYVLRHEIIALHSAGQANGAQNYPQCFNIAVSGSGSLQPSGVLGTDLYHATDPGVLINIYTSPLNYIIPGPTVVSGLPTSVAQGSSAATATASATVPGGGSGPTSRTTTTARTTQASSRPSSTPPATTSAPAGGPTQTLYGQCGGSGYSGPTRCAPPATCSTLNPYYAQCLN

SEQ ID NO: 16 - protein, 437 aa, Trichoderma reesei
QQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 17 - protein, 304 aa, Myceliophthora thermophila
HATFQALWVDGVDYGAQCARLPASNSPVTDVTSNAIRCNANPSPARGKCPVKAGSTVTVEMHQQPGDRSCSSEAIGGAHYGPVMVYMSKVSDAASADGSSGWFKVFEDGWAKNPSGGSGDDDYWGTKDLNSCCGKMNVKIPADLPSGDYLLRAEALALHTAGSAGGAQFYMTCYQLTVTGSGSASPPTVSFPGAYKATDPGILVNIHAPLSYTVPGPAVYSGGSTKKAGSACTGCESTCAVGSGPTATVSQSPGSTATSAPGGGGGCTVQKYQQCGGQGYTGCTNCASGSTCSAVSPPYYSQCV

SEQ ID NO: 18 - protein, 215 aa, Myceliophthora thermophila
HYTFPRAGTGGSLSGEWEVVRMTENHYSHGPVTDVTSPEMTCYQSGVQGAPQTVQVKAGSQFTFSVDPSIGHPGPLQFYMAKVPSGQTAATFDGTGAVWFKIYQDGPNGLGTDSITWPSAGKTEVSVTIPSCIEDGEYLLRVEHIALHSASSVGGAQFYIACAQLSVTGGSGTLNTGSLVSLPGAYKATDPGILFQLYWPIPTEYINPGPAPVSC

SEQ ID NO: 19 - protein, 323 aa, Myceliophthora thermophila
HGHVSHIVVNGVYYRNYDPTTDWYQPNPPTVIGWTAADQDNGFVEPNSFGTPDIICHKSATPGGGHATVAAGDKINIVWTPEWPESHIGPVIDYLAACNGDCETVDKSSLRWFKIDGAGYDKAAGRWAADALRANGNSWLVQIPSDLKAGNYVLRHEIIALHGAQSPNGAQAYPQCINLRVTGGGSNLPSGVAGTSLYKATDPGILFNPYVSSPDYTVPGPALIAGAASSIAQSTSVATATGTATVPGGGGANPTATTTAATSAAPSTTLRTTTTSAAQTTAPPSGDVQTKYGQCGGNGWTGPTVCAPGSSCSVLNEWYSQCL

SEQ ID NO: 20 - protein, 509 aa, Myceliophthora thermophila
QNACTLTAENHPSLTWSKCTSGGSCTSVQGSITIDANWRWTHRTDSATNCYEGNKWDTSYCSDGPSCASKCCIDGADYSSTYGITTSGNSLNLKFVTKGQYSTNIGSRTYLMESDTKYQMFQLLGNEFTFDVDVSNLGCGLNGALYFVSMDADGGMSKYSGNKAGAKYGTGYCDSQCPRDLKFINGEANVENWQSSTNDANAGTGKYGSCCSEMDVWEANNMAAAFTPHPCTVIGQSRCEGDSCGGTYSTDRYAGICDPDGCDFNSYRQGNKTFYGKGMTVDTTKKITVVTQFLKNSAGELSEIKRFYVQNGKVIPNSESTIPGVEGNSITQDWCDRQKAAFGDVTDFQDKGGMVQMGKALAGPMVLVMSIWDDHAVNMLWLDSTWPIDGAGKPGAERGACPTTSGVPAEVEAEAPNSNVIFSNIRFGPIGSTVSGLPDGGSGNPNPPVSSSTPVPSSSTTSSGSSGPTGGTGVAKHYEQCGGIGFTGPTQCESPYTCTKLNDWYSQCL

SEQ ID NO: 21 - protein, 851 aa, Myceliophthora thermophila
IESRKVHQKPLARSEPFYPSPWMNPNADGWAEAYAQAKSFVSQMTLLEKVNLTTGVGWGAEQCVGQVGAIPRLGLRSLCMHDSPLGIRGADYNSAFPSGQTVAATWDRGLMYRRGYAMGQEAKGKGINVLLGPVAGPLGRMPEGGRNWEGFAPDPVLTGIGMSETIKGIQDAGVIACAKHFIGNEQEHFRQVPEAQGYGYNISETLSSNIDDKTMHELYLWPFADAVRAGVGSVMCSYQQVNNSYACQNSKLLNDLLKNELGFQGFVMSDWQAQHTGAASAVAGLDMSMPGDTQFNTGVSFWGANLTLAVLNGTVPAYRLDDMAMRIMAALFKVTKTTDLEPINFSFWTDDTYGPIHWAAKQGYQEINSHVDVRADHGNLIREIAAKGTVLLKNTGSLPLNKPKFVAVIGEDAGSSPNGPNGCSDRGCNEGTLAMGWGSGTANYPYLVSPDAALQARAIQDGTRYESVLSNYAEEKTKALVSQANATAIVFVNADSGEGYINVDGNEGDRKNLTLWNNGDTLVKNVSSWCSNTIVVIHSVGPVLLTDWYDNPNITAILWAGLPGQESGNSITDVLYGKVNPAARSPFTWGKTRESYGADVLYKPNNGNGAPQQDFTEGVFIDYRYFDKVDDDSVIYEFGHGLSYTTFEYSNIRVVKSNVSEYRPTTGTTAQAPTFGNFSTDLEDYLFPKDEFPYIYQYIYPYLNTTDPRRASADPHYGQTAEEFLPPHATDDDPQPLLRSSGGNSPGGNRQLYDIVYTITADITNTGSVVGEEVPQLYVSLGGPEDPKVQLRDFDRMRIEPGETRQFTGRLTRRDLSNWDVTVQDWVISRYPKTAYVGRSSRKLDLKIELP

SEQ ID NO: 22 - protein, 373 aa, Myceliophthora thermophila
QSGPWQQCGGIGWQGSTDCVSGYHCVYQNDWYSQCVPGAASTTLQTSTTSRPTATSTAPPSSTTSPSKGKLKWLGSNESGAEFGEGNYPGLWGKHFIFPSTSAIQTLINDGYNIFRIDFSMERLVPNQLTSSFDQGYLRNLTEVVNFVTNAGKYAVLDPHNYGRYYGNIITDTNAFRTFWTNLAKQFASNSLVIFDTNNEYNTMDQTLVLNLNQAAIDGIRAAGATSQYIFVEGNAWSGAWSWNTTNTNMAALTDPQNKIVYEMHQYLDSDSSGTHAECVSSTIGAQRVVGATQWLRANGKLGVLGEFAGGANAVCQQAVTGLLDHLQDNSDVWLGALWWAAGPWWGDYMYSFEPPSGTGYVNYNSILKKYLP

SEQ ID NO: 23 - protein, 465 aa, Myceliophthora thermophila
APVIEERQNCGAVWTQCGGNGWQGPTCCASGSTCVAQNEWYSQCLPNSQVTSSTTPSSTSTSQRSTSTSSSTTRSGSSSSSSTTPPPVSSPVTSIPGGATSTASYSGNPFSGVRLFANDYYRSEVHNLAIPSMTGTLAAKASAVAEVPSFQWLDRNVTIDTLMVQTLSQVRALNKAGANPPYAAQLVVYDLPDRDCAAAASNGEFSIANGGAANYRSYIDAIRKHIIEYSDIRIILVIEPDSMANMVTNMNVAKCSNAASTYHELTVYALKQLNLPNVAMYLDAGHAGWLGWPANIQPAAELFAGIYNDAGKPAAVRGLATNVANYNAWSIASAPSYTSPNPNYDEKHYIEAFSPLLNSAGFPARFIVDTGRNGKQPTGQQQWGDWCNVKGTGFGVRPTANTGHELVDAFVWVKPGGESDGTSDTSAARYDYHCGLSDALQPAPEAGQWFQAYFEQLLTNANPPF

SEQ ID NO: 24 - protein, 464 aa, Myceliophthora thermophila
MGQKTLQGLVAAAALAASVANAQQPGTFTPEVHPTLPTWKCTTSGGCVQQDTSVVLDWNYRWFHTEDGSKSCITSSGVDRTLCPDEATCAKNCFVEGVNYTSSGVETSGSSLTLRQFFKGSDGAINSVSPRVYLLGGDGNYVVLKLLGQELSFDVDVSSLPCGENAALYLSEMDATGGRNEYNTGGAEYGSGYCDAQCPVQNWNNGTLNTGRVGSCCNEMDILEANSKAEAFTPHPCIGNSCDKSGCGFNAYARGYHNYWAPGGTLDTSRPFTMITRFVTDDGTTSGKLARIERVYVQDGKKVPSAAPGGDVITADGCTSAQPYGGLSGMGDALGRGMVLALSIWNDASGYMNWLDAGSNGPCSDTEGNPSNILANHPDAHVVLSNIRWGDIGSTVDTGDGDNNGGGPNPSSTTTATATTTSSGPAEPTQTHYGQCGGKGWTGPTRCETPYTCKYQNDWYSQCL

SEQ ID NO: 25 - protein, 360 aa, Serpula lacrymans var. lacrymans
YQVVRDYSGQNFFTGWDFYGNYDNLTLGNAVYLNQSEATKQNLAYINSEGRAIIKVDNTTDVAMGQNRSSIRMTSQDAYPIGSLWIIDLYHIPYGCSVWPAFWTFGPNWPNDGEIDIIEAINIMGNNQMVLHTTPGCTHSSTYNQLGANIGSDCSTPSGCVVAETQPNSYNSGFAAAGGGVWATQFDVTGVFIWFWSRPNVPESITQANSTSSIDITSWGTPSASYFANTCNITEFFSPQNLVFDITLCGDWAGTGYAYNATCGSSGPTGLCYNDCVVGPGSPRYDEAYFDISYVRAYTTEQPAPTTTTTSTSIPTSTTSTTNRQTSSSGAISTRNDSSRGLILLATVAVGIILGARFAL

SEQ ID NO: 26 - protein, 383 aa, Schizophyllum commune
YDLVRDYSGSSFFDRWDFYGYWDNLTLGDVWWLNRNDAFSQGLTYINNAGNAVLKVDNEHDVAWNYKRNSVRITSQDTYAVGSLWITDVVHVPYGCSVWGAIWTKGPTWPDNGEIDIFETINRMPINQYALHTTEGCMKDTPDNQVGTTVVEDCSQAAGCTVTENYENSAYTGFAEAGGGVWATQFDVSGIYIWFWSRPNVPASITQSTSTSGVDLSDWGPPSAAFPSTTCNITEYFTPQNLVIDITLCGNWAGLPETYAETCSGGTTGLCYNDNVIGSGANYNDAYFEIKNIRAYTTGGVAPTPTANYQGTIPTITGTSALHSSTSTVGTALIPTPWYYPGAALEMNSVRTATGAHLTFSTTYFAFYDMATTVDDSMQDVVL

SEQ ID NO: 27 - protein, 351 aa, Piriformospora indica
YNLAREYKGQNFFSAWDYYGKYDNLTNGDVIWVNQSVAVSNPQLTYINSAGNAIIKVDDTTTVPYNEKRNSVRLTSLDKFNLGTVMVFDALHVPYGCSVWGALWSQGINWPAGGEIDIFEAVNLMTANQMALHTESGCAQADGVTQTGITQVKNCDNNGSNGAGCTVLDANTNSYGEPFAAAGGGVWVTEFAKTGINIWFFSRANVPASLSTDTIDVSTFGTPSASYPASSCDPAKYFSEQQIVIDITLCGDWSGVKSVLESTCPALNGTNTCYTTYVLDPKNYVNAYFELASVKIFASDPSAVVTAAGVTQTVSATGSPHPNAASGMGPTGAVAFGAFALTAMLGLVLQF

SEQ ID NO: 28 - protein, 355 aa, Postia placenta
YSLVKTYSGSSFFDGWTFYGNYDNTTDGDVTYVNQSLATSDKLAYVDSSGQAIVKVDNSSFVVYNDKRNSVRITTQDYFPLGSVILFDATHLPYGCSVWPGFWTKAAQWPEGGEIDIVEGVNGMTSNQMALHSTGGCSATSSANASGTIGPTNCSAAAGCTYTETKADSYGAGFASAGGGLWATLFDSTGISIWFWGRADIPSSISSAGSSLSVADWGTPSANYPASSCDIAEFFQPQQIVIDITLCGDWAGLTSIYPETCPIVGASTANASSCYLQNVINSGNQTALSEAYFAMNSIKVYNANGTVVSASGASSSVSPTSTAAQGSKTTSGAGSRGALSVFAAGIGALAAWTLL

SEQ ID NO: 29 - protein, 334 aa, Postia placenta
YSMVKEYQGASFFDDWNFYNNYDNLTSGNVNYLSAKTAGQDQLAYINDAGNAIMKVDNTSTLNVGANRNSIRISTKDHFTVGSMWITDMVHVPYGCSVWPAFWSSAQDWPSGGEIDTFEGVNQVTMNQMALHTAPGCTHPANATQTSKLVNSTDCSIDANNNEGCVVTTPTTSSYGQGFAADGGGMFVTEFAEDGISVWFFNRSSIPSALSGNASTVNPSDLGTPTANWPSSSCSPAQFFDPQALVFDITLCGEFAGNSVIFQETCSGVCYDDWVLGPPSNYDNAYFEVQYVRVYGATGELTVISGARPSTPYRSVVAAAALAVAAVGGALLAL

SEQ ID NO: 30 - protein, 375 aa, Laccaria bicolor
YDIVQDYSGSSFFDKWDFYGNWDNLTLGDVWWLGRQDAFAQGLVYVNNAGNAILKVDDVSNVALNQKRNTVRITSQAAYNVGSLWIADIVHLPFGCSVWPAFWTKGTLLWPDGGEIDIIEGINLVSQNQMALHTLPGCYHNTTPPNQMGISDPTKVDCSQPSGCVVSESAPNSFGQGFANAGGGVFGVQFDVAGVFIWFWSRPSIPASIANANATSSINVSDWGAPSASYPASEPCNITQFFTPQNLVIDITLCGIWAGLPAQYLPQCSGAGPTGICYNDNVVGPGGRYSNAYFEIKYVRAYTTGGIAPTPTAAAHALLSQAVTSTIKTTSPAGTVLVPSPLFFPGNTARRGFEVQGWRLTLVGIIVVMTTYLVW

SEQ ID NO: 31 - protein, 369 aa, Schizophyluum commune
YYPLREYSGGSFFNGWDFYDNVDNTTWGNVRYVDQTTAIEQGLAYVTDKGNAVIRVDDETYIPGGWDFTNRQSVRITTTDTYPLTGLVIIDLVHMPYGCSVWPSFWSFGPSAGEWPAGGEIDIIEGVNLVGHNQMALHSTEGCFQANNSGGTGQTLERDCSTPTGCAVRENKNDSYGQAFNEAGGGVFALQMAQSGFYIWFWGRDDIPDSISSANSQSTMDTTKDWGTPSASYPASGCNDTLWKYFAPQQLVLDITLCGNWAGLPTVYGATCVNQYACLTDNVVGDGSNYAEAYFEIRWIRTYTGDKALVPSFTPSPTFSLSVSTSSSRSSASATSSSNDSESVLPTPWLSMILYCLLGAVLTLCCISL

SEQ ID NO: 32 - protein, 310 aa, Serpula lacrymans
YNAVKEYSGSTFFNDWTFYNNYDNLTNGDAIFVSASEGASDQLAYVDSSTLHAIIKVDNTTTVPYNQKRNTVRITSNDSFAIGSVWVADMYHVPYGCSVWPAWWSQAPSWPAGGEIDTFEGVNMMTMNQMSLHTETGCMVENQNQTSTLIQSTNCSASANGNQGCIVQDPSGSSYGAGFAGVGGGAFVTEMAESGINIWFFPRSQIPSSLTSNASTIDTSTFGTAVGNWPSGGCNTTEFFQPQQLIFDITLCGAGSPATFNATCTGVCYNDYVIGPASNYNEAYFEIGYVRVFGTEGADTVISPSGSSGV

SEQ ID NO: 33 - protein, 353 aa, Coprinopsis cinerea
YHVTKEYSGSTFFNDWDFYGSHDNLTNGDVEFISASEARTPSSLAYVDPNTNRAIVKVDNTTEVPYMEKRKAVRITSKDAFPVGSVFITDIHHAPWTWAAADDNWPAGGEIDIFEGINQETRSQMGLHTEPGCVQTSPNQLTTEVRSTDCHGPNNEGCIVSNTDPASYGPAFAAAGGSVFVTEFAETGISIWFFSRANIPGSITPTATSIDTATLGTPMGHWPATGCDINRFFRPQNLVFAITLCGDFARPPDIFGRTCEGQCYLDFVIQDPGYYSNAYFDIAYVKVLSTEPSSSTDIASSPAASRFSTVTAPNGDASETDASESAAFGRSLLVSGVGLGALILIPAALVYLI

SEQ ID NO: 34 - protein, 202 aa, Moniliophthora perniciosa
YEPIREYSGQDFFSEWDFTSGVDSTTSGNVQYLDQSAATQQRLAFVDSSSGHAIVRVDNTTKLEDGPSVHRNSVKIISKDAYPIGSLIIIDAVHIPYGCSVWPAFWTLGTELGWPHAGEIDIIEGINGMTSNSMVMHTDPGCTQSANVSQSGKTLDTDCGTAVGCKVEDTKPNSYGPGFAQAGGGVFATQIDVSGVFIWFWS

SEQ ID NO: 35 - protein, 334 aa, Laccaria bicolor
YTMVKTFAGSTFFDDWKFYNNYDNLTNGDAIFVSSSVAASSQLAYVDPSTKHAIIKVDNTSTVPYNQKRNTVRISTNDKFSVGSVWTVDMLHVPYGCSVWPAWWSQAPAWPTGGEIDTFEGVNMVTNNQMGLHTLAGCKQVSQVQSSTLVNSTDCSYLTNSNEGCITTNPSTASYGAGFAQAGGGMFVTEFAETGISIWFFSRANVPSVLSSNSSTIDTSTLGTPVGNWPAAGCKIDTFFAPQNLIFDITLCGDFAGAANVFAETCPGTCYNDYVVGNGSNYATAYFEIASVNVFSKTGTNTIVTGNSASALCLSLSTLASWLGVMGGIWLFLA

SEQ ID NO: 36 - protein, 364 aa, Cryptococcus neoformans var. neoformans
YPLVESWHGKGFFDGFTFPVETYDNTTNGDTFWATPANTSLLYTTSTGATILKVDNRTFVPYPEKRYAPRLLSKSAYDLGTVWVMDAVHLPYGCSVWPAFWTQGPSWPAGGEIDIIEGINLQATNMIALHTSGASSCTIPTTSPSPFSGTVSYPNCDNSQNYGSGCTVYDRNTNSYGREFAEAGGGVYVAEFAKDGIRVWFMTRSAIPTAIQVNATQIDTSTLGTPVAEYPSSSCDIANLFGPQTLTINIALCGDYAGLPSELQRTCPALVGDATCYTTYVINDGSTTYAQAYFEINYVNVYSSNPSSVTTISPSGPTSTSTLTSTSTTASTSAAGTREGGATKAERQLLLVAVGSLVSLFLFW

SEQ ID NO: 37 - protein, 361 aa, Rhodotorula glutinis
YTLQTAYQGDRFFDGWDFWNNRDNLTNGAVNYVSKAASSEVAYTNSAGNVVIKVDNSTRLASGVNALRDSVRITTNDAFDVGSLFVMDALHVPYGCSVWPAFWAHARSWPSGGELDIFEGVNLQQTNQVAMHTVAGCYAANSTVNVTATGDMTFSNCDYTVAANHGCTFQDARNASYGADFAAAGGGIYAAEFSSDAISVWFFPRAEIPADLRSVNGTPDPSSWGIPMAYYPSSACNINQYFAPQQITINIALCGDWAGQPGVFSPICGTGNCADYILDPSHFDTAYFEIASVRIYEGGVNTRVSGGAQAASGVIGAIGGSASGTSSADGGRWRASGRALGAVGLAAAVSVLSGVGLVVGL

SEQ ID NO: 38 - protein, 374 aa, Serpula lacrymans var. lacrymans
YNAVKEYSGSTFFNDWTFYNNYDNLTNGDAIFVSASEGASDQLAYVDSSTLHAIIKVDNTTTVPYNQKRNTVRITSNDSFAIGSVWVADMYHVPARSGPLGGGKPVRILHTTLELVVMRLFTHSQAPSWPAGGEIDTFEGVNMMTMNQMSLHTETGCMVENQNQTSTLIQSTNCSASANGNQGCIVQDPSGSSYGAGFAGVGGGAFVTEMAESGINIWFFPRSQIPSSLTSNASTIDTSTFGTAVGNWPSGGCNTTEFFQPQQLIFDITLCGDWAGSPATFNATCTGVCYNDYVIGPASNYNEAYFEIGYVRVFGTEGADTVISPSGSSGVGSGGVPGSTGSPTTGAAVGRGEGWGLVAVVAAVVVGLAVGLAV

SEQ ID NO: 39 - protein, 334 aa, Schizophyllum commune
TYDLVKEYSGESFFDGWSFFGNYDNLTNGDAIFLSADENNDAKLAYVDETTGRAIIKVDNTTNVPYNEKRNTIRIASEERYDIGSVFVADFYHVPYGCSVWPAWWSQAPNWPTGGEIDTFEGVNMVTMNQMALHTEDGCKQVSPSQSSTLVNSTDCNKDVNENSGCVVTDPETDSYGEAFAKAGGGVWVTEMASSGISIWFYSRSEVPDAIKNNDSSIDTSSLGTPVANWPTGGCDIDTFFQAQNLIFDITLCGDFAGADNVFSQTCSGKCYEDYVVGNGSVYATAYFDIAAVRVFGQSGTNVVVDGDSGALPNAKWGLGSLVAAVGFLFGVAL

SEQ ID NO: 40 - protein, 356 aa, Postia placenta
AAFDLLHDYSGSSFFTGWEFYGKWDNLTLAGNVTYQTALLATEYQLVSVNEAGNAIIRVDNRTTVSVGERRNSVRLTSSEFYDFGSLWIIDLLHIPYGCSVWPAFWSTAPNWPDGGEIDIIEAINLATSNQMALHTTAGCTHYPQVNQTGYNIDTDCGTGSGCTVGIPANNSYGPGFASVGGGVYATWFDESGIFMWFWSRPDVPDSIANAGANSSMDVSTFGIPTASFPTNTSCNITQFYKPQQLIFDITLCGDWAGVPGIYDSQCYNAGPNHDCYLDCVVGDGSNYDDAYFEVRYVRTYSDRPVTPSTTGDPTTQTSQASTQPHNAARSTRAEWRWWLGLPLAAICADMLLRLL

SEQ ID NO: 41 - protein, 307 aa, Postia placenta
PWKVKQSYEGDSFFDGWSFFTASDPTDGTVQYVDQSTAQSANLTSINSAGNAIMRVDTTAKISGNRQSVRITTNYNYTGALVILDSVHMPTGCGTWPAFWSNGPNWPAGGEIDIVEGVNTYTNNQATIHTNPGCTIPSSNSTVLGITGDVTGGTNCAAAETGNAGCGIRSTSNTSYGAGFNEIGGGVYAMEWVDSGISVWFFPRSSIPSDITAGAPQPSGWGTPMANWPSTDCNPSTFFYQHSAIFDTTLCGQWAGNVWSDTGSPGQSQSCAQITGTSTCAEYVQNNGAAFADAYWEVKSVKIYQTS

SEQ ID NO: 42 - protein, 341 aa, Postia placenta
SGSSSVASLGAKPSATATSSSAPAASSSAVASSPWKLKQSYEGNSFFAGWSFFTDTDPTGGTVDYIDGSAAESANLTGINSAGNAYLKVDTTPVITSGYRRSVRITTDFTYTGALVVLDAVHMPTGCGTWPAFWSNGPNWPDGGEIDIVEGVNDYTNDQVTLHTNTGCSLPTSNATVLAIAGDIVGSTDCSVSGTGDAGCGIRASQTNSFGAAFNDIGGGVYTMQWDDTGVSVWYFTRSTIPADITAGAPQPSGWGMPIANFPASSCNPSQFFYDHSAIFDTTLCGAWAGDGWTASGIPGQEQSCAQRTNTATCAEFVANNGAAFEQAYWEVKSVKIYQTS

SEQ ID NO: 43 - protein, 308 aa, Postia placenta
PWKLKQSYEGNSFFAGWSFFTDTDPTGGTVDYIDGSAAESANLTGINSAGNAYLKVDTTPVITSGYRRSVRITTDFTYTGALIVLDAVHMPTGCGTWPAFWSNGPNWPDGGEIDIVEGVNDYTNDQVTLHTNTGCSLPTSNATVLAIAGDIVGSTDCSVSGTGDAGCGIRASQTNSFGAAFNDIGGGVYTMQWDDTGVSVWYFTRSTIPADISAGAPQPSGWGMPIANFPASSCNPSQFFYDHSAIFDTTLCGAWAGDGWTASGIPGQEQSCAQRTNTATCAEFVANNGAAFEQAYWEVKSVKIYQTS

SEQ ID NO: 44 - protein, 351 aa, Coprinopsis cinerea
SRNRSEPLERRNQYLDRNRDGTPFVWLLEDDYKGHDFFDHFEFFNWTDPTKYVSREEAFARRLAYVQDDGIVVMKADDTSHLPRGEFRSSVRINTIKRYTTGLFILDLNTAPWGCGVWPAWWSTGDNWPVSGEIDIIEGVHDNEHNQIAWHTEPGCVLDTEESFTGNVSIKSGGPAVECNAHINQNAGCSITEWSRASYGPYFDEQGGGVFAMKWDENGIAVWSFYRAAIPKDITEGNPNPRNWGDPSALLGPGKCNIMEYFRNHTVILNITFCGDWAGNSYATSGCPGTCPDRLMDPANFVNATWSINSMKVYRKQPIYAEVVDPNKSAASRNVLGSLALVPLVGAALMN

SEQ ID NO: 45 - protein, 339 aa, Coprinopsis cinerea
TIPPRCVAIEPRNAFYDRNRDGSPFVWLLEDTYQGEDFLKNVNFFSGHHDPTRYVAREEAYQNRLAYVADNGNVILKADDTNQLPFGENRTSVRVNTVKDYSGGLFILDLDRAPWGCGIWPAWWSTAVGTGWPALGEIDIIEGVHDNQHNEMAWHTAEGCLLDTEEDFTGNVSIKHNGPATNCWAHLPGSNNVGCSITESSRSFFGPYFESQGGGVFAMKWDENGIAIWSFYRAAIPQDITNGTPNPSSWYKPSALLGPKKCDIEKYFRNHTIILNITFCGDWAGNTYEAAGCPGSCRERLMNPANFVNATWSIRSLKVYRKQLIHAEIHGGAVSALAG

SEQ ID NO: 46 - protein, 354 aa, Laccaria bicolor
HLWPSRSLYVASGNSINYNPNGSSFLWLPQDSYSGQTFFDLWDFFTGADPTNGQVNYVNETVARQTGLVYVQDNGIVIMKADNTTSLASGVYRNSVRISSQAQYNTGLFILDLNRAPWGCAVWPAFWTVGGNWPYDGEVDIIEGVHDNEHNQVAWHTAPGCTLDTTANFTGTISSSDGVNHTDCNAFINSNSGCGMTEWSRASYGPYFDSQGGGVFAMKWDENSIAIWSFYRVAIPKDVIAGTPNPSGWGAPSAVLEPSDCNLGQLFANHSIVFDITLCGDWAGNSYATSGCPGTCEQRLMDPANFQNASWSINSLKVYRKVLLSARVTNSVTSKLTMEFTTLYLMILLGGLLL

SEQ ID NO: 47 - protein, 342 aa, Schizophyllum commune
RRFDAQQLDVNANGSTFLWLLEDDFSGDDFFNNFGFFTGEDPTHYVDANTAFGSGLSYVQDDGIVVMKGDNTSWLGDGEYRKSVRISSYKQYNTGLFILDLNKAPWGCAVWPAFWTLGSGTWPQTGEIDIIEGVHDNEHNQVAWHTASGCYLDPTASFTGTVVVQNNTNCDGSVNSNAGCAIAEWSRASYGPYFDAQGGGVFAMKWDENGIAVYSFYRAAIPDDINAGSPNPSGWGKPVAFLSPDSCDPIKYFTNHSIIFDITFCGDWAGNSYATSGCPGECSDRLKDPANFVNASWSINSLKVYSKQPLNGHLNGAPPRYSEHVAPTLLAFGLAVLSWHLW

SEQ ID NO: 48 - protein, 354 aa, Serpula lacrymans
VRNPGSELSNKRDVSYDATTNISTVLWVIEDTYEGQTFFDTFHFYTGPDPTNFVDEQTAYNSSLAYVTPDNKIIMQGDNTTWLPQGVNRSSVRISSQAVYNTGLFILDLDMAPWGCAVWPAFWTLGSGVWPYNGEIDILEGVHDNSHNQVTWHTAPGCTLTPTTNFTGTIDQINGVDNLECNSLINDNAGCSVTEWSNASYGPYFDAQGGGAFAMKWDEEGIAVWSFYRAAIPQDIVQGEPNPTNWGSPVASLAPQTCNMTEYFANHSIIFDITFCGDWAGNSYATSGCPGTCPERLMDPANFVNASWIINSLKVYKKATLSGGASNGVAEYPISAVMGWIVIGLILNFGLLTL

SEQ ID NO: 49 - protein, 349 aa, Laccaria bicolor
QTHPSRNLEKRAGINTNSNGSTFLWLTQDVYAGETFFDRWDFFDYEDPTNFLNRSEAIRRNFTYTESNGTVVMKADMESVLPSGVNRDSIRIQSKARYNSGLFILDLTRAPWGCAIWPAFWTTNENWPWNGEIDIIEGVHDNQHNQIAWHTAPGCYLDPTLNFTGTIVSQSRQNCDGTINDNAGCGVTEWSRASYGPYFESQGGGVIAMKWDEDGIAIWSFYRAAIPGDVIAGAPVPSTWGPPSAILGPAKCNITNFFHNHTIVFDITFCGEWAGNSYATSGCPGTCAQRLMDPANFVNASWHINSLKVYKKVLLNGVKPTSDSIIGIDASRRGYFLLLMTLLLVLLFQ

SEQ ID NO: 50 - protein, 358 aa, Serpula lacrymans var. lacrymans
VRNPGSELSNKRDVSYDATTNISTVLWVIEDTYEGQTFFDTFHFYTGPDPTNFVDEQTAYNSSLAYVTPDNKIIMQGDNTTWLPQGVNRSSVRISSQAVYNTGLFILDLDMAPWGCAVWPAFWTLGSGVWPYNGEIDILEGVHDNSHNQVTWHTAPGCTLTPTTNFTGTIDQINGVDNLECNSLINDNAGCSVTEWSNASYGPYFDAQGGGAFAMKWDEEGIAVWSFYRAAIPQDIVQGEPNPTNWGSPVASLAPQTCNMTEYFANHSIIFEDFQDITFCGDWAGNSYATSGCPGTCPERLMDPANFVNASWIINSLKVYKKATLSGGASNGVAEYPISAVMGWIVIGLILNFGLLTL

SEQ ID NO: 51 - protein, 352 aa, Serpula lacrymans ver. lacrymans
HEPPGPVLRRDGNSTSSNASDVLWVIEDNYEGQTFFDRFDFYTGADPTHGMVDFVDQQTAYSSGLAYVTSNNKVIMKGDNTTTLAMGANRGSVRISSQAIYNTGLFILDLDMAPWGCAVWPAFWTLGGGTWPYSGEIDILEGVHDNQYNQVTWHTAPGCMMTSTVNMTGTIDQINGTDNLDCNALINSNSGCAVTEWSRASYGPYFDSQGGGVFAMKWDDDGISVWSFYRAAIPTDIVQGNPNPAGWGVPVASLSPEACNPTQYFVNHSVIFDITFCGDWAGNSYTTSGCPGTCEERLVDPANFVNASWIINSLKVYKRASVSVSNDATQYAVLGPALIGWILLSIVLNVGL

SEQ ID NO: 52 - protein, 362 aa, Piriformospora indica
RAERGRQFLDVRRSIMMQKRSYNKTVYVSAKTYQGANFFDEWDFYSGQDYTHGYVDYLTKEQAFTKGLAYVTEEGRANMHVDNWTVLTVDDINNGKYRPSVRISTTAKYNHGLYILDVAKAPFGCSTWPAYWSTNENWPRDGEIDIIENVHASLSNQVSWHTLPGCNLVTSGNYTGTALNTICDSNYMSNTGCNIVDPSVASFGPVFNEKGGGVFAMKWDDKSIDVWFFYRAAIPDNIIQGLPDPTTWPTPSASLSSQGCPIDQFFRNHMFIFDTTLCGDWAGTSYSTSGCPGSCAEQVANPSNFVNATWSINYLKVYNKTVINTSYLDSSATQGTSSTNAWTSIYLLLITSLAARLLSVAL

SEQ ID NO: 53 - protein, 345 aa, Piriformospora inidica
RARLHKRAVNKTVWVSAHSYEGPTFFDGWEFWAYPDPTNGLVQYVNRDVAFAEGLAYITPEGRANMHVDSKTVLTLEEVTSRRKLRKSVRLHSKILYTHGLFLLDVAQAPYGCGTWPAYWMTGFNWPADGETDIIENVHSNASNQVAWHTSPGCYLTSPGNYTGYAGSLNCDASINYNKGCGIVDQSIASFGQTFNEKGGGIYAVKWDSDSIDVWFFYRSAIPSNILEGLPDPATWPLPSASLSRPGCDIDKYFKNNMIIFDTTLCGDWAGTSYAAAGCPGTCEERVTNPNSFVNATWSINYIKVYNKTIINTWYLEAGARLRAHAGLFLVLVTTSLVIFGVCLC

SEQ ID NO: 54 - protein, 310 aa, Schizophyllum commune
DGFEYRAKHRAHGNSTLQTRKTWTLADKYQGQDFLDKWDFFSQADPTHGSVNYQDKSSAQSKNLAYVQDDGAFVMAVDDQTQLSVGDKRDSVRISSKKSYTQGLFIADIWAMPHGCSVWPAWWTVGPNWPNGGEIDVLEGVHDQHVNQYTLHTSQGCSIDTSVDVTGTLGNQQCAVGGGDNTGCAFTDSDPTSYGNPFNVLAGGVYAHTWTDEGIKIWHFPRTSIPADITSGSPNPDSWGAPAAFFSANNCDMGSHFYDHVLTFDITLCGDWAGSTYGSAGCPGSCAERVANPANYKYAKFKINYVAVYQ

SEQ ID NO: 55 - protein, 319 aa, Schizophyllum commune
DGFVYRSRPQEHHSGSNSTILQTRKTWTLADKYEGQAFFDQWNFFEWGDPTHGRVNYQNKENAQNKGLAYVQGDGKFVMAVDDKNWVGVGSNRDSVRIGSQKAYTQGLFIADLQAMPFGCSVWPAWWSVGPNWPNGGEIDVLEGVHNQKVNQYTLHTSPGCTIDTGVQATGQIGNQQCAVGGNDNTGCFFTDTNDNSYGQPFNAAGGGVFAHTWQDDGIKIWHFARDSIPGDISSGNPNPDGWGEPVAYFSSNTCDIGSHFYEHGLTFDITLCGDWAGATYSQAGCPGSCDERVANPDNFHGKSWLTLSVIRNLINDFN

SEQ ID NO: 56 - protein, 316 aa, Piriformospora inidca
TSRQNSTSAYNLVDSYEGSTFFDGWDFFEYADPTHGMIRYVSADEAKSSNLAYVRDDGVAVMTVDTTTTLAVSDSEQRNSVRITTKKSYGQGLFIFDILKAPHGCSTWPAAWLVGPDWPSGGEIDVVEGVHENVYNQMTVHASAGCQLDATKSLAGGVTLRADQNPLEMFTGTVLETDCDATINSNAGCGIMDYDTTSYGAGLNDAGGGVYATLWDNVGVRIWFFKREDVPADITGSTPDPTTWGTPRAYWAASSCASSFFNNLSIVFDIVLGGDWAGATYSAAGCPGTIQDYVANPSNFANANWAVNSVRVYQTS

SEQ ID NO: 57 - protein, 304 aa, Lentinula edodes
APLRHRRSKMLNRRTTYTLSDTYEGDSFFDTWDFFTDTDPTEGYVAYQSRENATAMGLAKVENGVAIIAVDSTSTLPSGTNRASVRISTQKTYNGGLFIYDVPFMPVGCGTWPAIWSTSTGTWPNDGEIDMIEGVHESTENQITMHTNAGCTLATGQAITGTVSGTTCESSDSNNNGCATMDTTPSGWGTAFNAAGGGVFAKLWDDTGVKIWHFSRGNIPADITSKNPDPSTWGNPVSFLPSGDSCNVAEHFKDHSLIINITLCGQWVGATFSCGGTCQSAVMDPSNFVDAQWKVNSILVYQPS

SEQ ID NO: 58 - protein, 298 aa, Phanerochaete chrysosporium
ATYHLEDNWVGSAFLSTFTHEAIADPTHGRVNYVDQATALAKNLTYASGDTLILRADHTTTLSPSGPGRNSVRIRSIKTYTTHVAVFDVRHMPQGCGTWPAAWETDEGDWPNGGEVDIIEGVNDQSPNAMTLHTGANCAMPASRTMTGHATNNNCDVNTDGNTGCGVQAPTANSYGPSFNANGGGWYAMERTNSFIKVWFFPRNAGNVPNDIASGPATINTDNWGTPTAFFPNTNCDIGSHFDANNIIINLTFCGDWAGQASIFNGAGCPGSCVDYVNNNPSAFANAYWDIASVRVYQ

SEQ ID NO: 59 - protein, 298 aa, Postia placenta
ATYNIDTTYIGTDFLNSWTHETLLDPTGGRVTYVDQATALADNLTYANGDTLIMRCDDTTVLSADGPGRNSVRIKSNAQYTTHVTIFDIRHMPQGCATWPAAWETDDTDWPDAGEVDVIEGVNDQTPNTISVHVGSTCSMPSSRDESGTPGSNNCDVNTDGNSGCGVSNPTDNSYGPDFNSAGGGWYAMERTSSVINVWFWTRTNGGVPSDVSSAASSIDTSNWGQPVGYFPNTDCDIGSVFGANNIIFDLTLCGNWAGLSSVYSAAGCPGDCVDYVNNNPSAFSEAYWDVASVIVYT

SEQ ID NO: 60 - protein, 317 aa, Laccaria bicolor
ATYLLTDNIVGKTFYSNFDWEAIPDPTHGRVNYVNSATSSSQNLTFATSDTFILRTDFKTVLDPNGPGRNSVRIRSKKTYTTHVAVFNMRHMPQGCGTWPAVWETDGANWPNGGEIDILEGVNDQAPDLVSVHTSPGCTMPSSRTMTGTPTYLDCQTGANSNAGCGVKLSTTLSYGPAFNKVGGGWYVIERSPTYMKVWFWSRRDTSVPAEVANGGQYVNPDTWGTPAAYFPNTSCDFPSHFDAHSIIINLTLCGDWAGATYGQTSCPSTCVGYVNNNPSAFTDAYFDFASLRVYGATVPLTKRGVDVHGRAHRREF

SEQ ID NO: 61 - protein, 299 aa, Serpula lacrymans var. lacrymans
GTYTVSDNIVGDDFYSAFTFEAIADPTDGRVNYVDEATAQSLNLTYTTSNTFIMRADDTTVLTASGPGRNSVRIKSNTAYTTHAVIFGMNHMPEGCGTWPAVWETDESNWPDGGEVDIVEGVNNVVPNQSTLHTSPDCTIPSSGGMLGTVVGTDCDATVNGNAGCGIQYTEDDNSFGPDFNNVGGGWYAMERTNDAISVWFWERSSSSVPAEVSSGASSIDTSTWGTPAAYFPDTDCDLATHFDANNIIINLTFCGDWAGSSSVYAASGCPSTCVDYVNDNPTAFTNAYFEFASINVYT

SEQ ID NO: 62 - protein, 298 aa, Schizophyllum commune
AAYVKSESFVGQSFYDGFDFQAIADPTHGRVNYVDAQTAKDKNLTYASADSFVLRVDSTTVLDPAGAGRDSVRIRSKNTYTTHVLVADVRHMPQGCGTWPAIWETNEATWPAGGEVDILEGVNDVVPNAATLHTSPGCTMPATRDELGTPTQPDCDTAVNGNAGCGVKFSDATSFGPALNAQGGGWYAMERTDSFIKVWFWPRGAADVPIGVSEGHDDVDTANWGTPQAFFPSDTCNIAEHFDAHNIIINLTLCGDWAGATFNTDGCSGVCVDVANSQPDAFKDAYFDIAAINVYKAQ

SEQ ID NO: 63 - protein, 298 aa, Coprinopsis cinerea okayama
NVYFMSDNIQGAGFYNAFEWENIADPTHGRVNYVDMETSKQQNLTFAVDDKFILRADSTSFLDPNGPGRNSVRIRSWKTYTTHVAVFDVKHMPQGCGTWPAIWEVQGDNWPNGGEVDILEGVNDEGPNAATLHTSPGCRMPASRSDQRGIRVLDNCDATINSNIGCPVQFPTPQSYGPAFNEIGGGWYAMERSPTYIKIWFWARDDPSVPDEVKYAAGVVNPDHWGLPTAFFPDNQCNMNEHFGPHNIVINLTFCGDWAGQTYEQSGCPGTCVDFVNNNPSAFEKAFFDLRGIRVYQK

SEQ ID NO: 64 - protein, 299 aa, Piriformospora indica
SNYRITDNWVGTSFLSAFSWENIADPTHGRVNYLPQSASLARNLTYAHGTHFIMRADSATVLSPSGPGRDSNRIMSTKSYGHNTVLVADIYHMPGGCGTWPALWTTDVNTWPNGGEIDILEGVNDKSPNAITLHTSENCMMPASRLQTGTSTQLDCYWMTNGNAGCGVLERKANSYGPSFNAAGGGWFAMERTPNFIRAWFWSRQDSSVPFDVRNPGQATVNTDGWGTPTALFPNTNCDISSKFAPHKVIINLTFCGDWAGNTYLNDGCPGNCIDRVNNSPGSFTEAYWDIGAIRMYGL

SEQ ID NO: 65 - protein, 298 aa, Postia placenta
GTYNIDKTYIGEDFLNTWTHEAISDPTHGRVDYVTQATALAENLTYANGDTLIMRADATTVLSASGPGRKSVRLQSQDSFGTHIVIFDVRHMPVGCGTWPAAWETGPNWPANGEVDVIEGVNDQGPNLVSLHVATTCSMPSSGRDMSGTAGSLNCDVNTDGNSGCGVNNPTSNSFGHDFNNAGGGWYAMERTSDEVKVWFWSRQDSTVPGDVQSGADEVNTNNWNQPVAYFPSTDCDIGNEFGKNNNLIFDLTFCGDWAGGSSYAAAGCSGTCVDWVNNHPSSFHDSYWDVAAVRVYT

SEQ ID NO: 66 - protein, 296 aa, Laccaria bicolor
ATYTLSDEIVGTGFYNSFDWQNISDPTHGRVNYVDKTVSQSLNLTFASSNTFILRGDSKTVLSANGAGRNSVRIQSKKTYRTHVAVFDVRHMPEGCGTWPAVWEVAGSGWPNTGEVDIVEGVNNQGPNAVSLHTSAGCTMPASRQQTGTSTQLDCNTNINGGTGCGVLLSSQKSFGPTFNQNGGGWYAVERSATAIKVWFWARTECSVPNDVQTGNLAVDPSNWGVPSAYFPNTTCDLAARFSDHYIIINLTFCGDWAGGAYAQSGCPSTCVDYVNKNPSSFVNAHFDFASLRVYQ

SEQ ID NO: 67 - protein, 294 aa, Laccaria bicolor
AIYSLSDEVYGPGFYSFFEWEAIEDPTHGRVTYVDKPTSIHQNLTYATSDTFILRTDFKTVLDPNGPGRNSVRIRSKKTYTSHVAVFDVRHIPQGCGTWPAIWETLEANWPNGGEIDIMEGVNDQGTNQATLHTSPDCLMPTSRTMAGTPTYDTCDVTLNFNAGCGVKFPTASSFGPAFNTNGGGWYAMERSQTYFKIWFWSRNDCDVPSDVAHARSFVNPDAWGTPTAYFPNTFCDFSTHFDPQNIIINLTLCGDWAGSTYSQGTGCPLTCVDHVNYNASAFTDAYFDFASIR

SEQ ID NO: 68 - protein, 297 aa, Serpula lacrymans var. lacrymans
ATYSRTSNLVGQGFMNAFYWQAISDPTNGRVNYVDDATAQRSGLVSVSGNTVTLRADDKAVLSANGPGRDSFRIESNAQYTTHVAIFDIGHMPEGCGTWPAVWEVGANWPNEGELDIIEGVNNESPNESTLHTSAGCTMPNGRDMSGTSTGSNCDVDQTNNMSCGVKLSASDSFGPSFNNNGGGWYAMERTSSAIKIWFWDRYSGSVPSDVKYAGNSINTGAWGTPAAYFPDTDCDFASHLGSHNIVINLTFCGDWAGSSDVYASSGCPSSCVDYVNNNPTAFSNAYFEFNALNIYE

SEQ ID NO: 69 - protein, 351 aa, Postia placenta
GSYSLSQSNVGSDFLSNFQWENITDPTNGRVEYVTQSTALAENLTYTSSDTFIMRADYTTTLDASGPGRKSNRIKSNTKYNTHVAVFDIRHMPQGCGTWPALWEADDTVGTSAGEIDILEGVNDVSPDSVTLHTNGTCTMPSNRTMLGTALSNDCSSSASVENGNNGCPVDAPYTSSYGTVFNTYGGGWYAVERTSEYIRVWFWSRNGTTTPSEVSSGASNINTDSWGTPIAYFPDTSCNLKDLFGNHNIIIDLTFCGSWAGEAFGPAGCPGNCTDYVNSNPSAFENAYWDFAAARVYLPSSSASNSSNATSTSNFSAASSSSTSSGSYVTQALSVPVLLSMLIGAVYIMN

SEQ ID NO: 70 - protein, 296 aa, Schizophyllum commune
KSYHLSESYVGSGFYSGFDFQAITDPTHGRVNYVDMYTAQGRNLTYTTGDTFILRGDSWSYLRPNGPGRDSVRIQSKKTYTNHISVFNVRHMPQGCGTWPAMWYADVNNWPGAGEIDVVEGVNDISPNAATLHSTAGCTMPGGRDMQGSPTQSDCNVNVNGNAGCGVRMSTPLSYGPSFNANGGGWFVTERKSTAISVWFWARNDPSVPAAVRDRRSDIVSGDLGTPQAYFPNTNCNFGSHFGALRIIINLTFCGDWAGNVYNNDGCPGSCIDRVNNNPSAFGEAYFNIANIDIYV

SEQ ID NO: 71 - protein, 313 aa, Laccaria bicolor
NTYQLSNKIVGNDFYDNFNWEAIDDPTHGRVTYVDQPTSKILNLTFTSTDTFILRTDFTTVLDPKGVGRQSVRIRSNAAYTTHVAIFSIRHMPQGCGTWPAIWTTAPNAWPNDGEIDILEGVNDQAPNLSVLHSTQGCTMPNNRTMTGTPTSTDCVTTDTSNAGCGVNFPTSFSYGPSFNSVGGGWYVMERNEKKITVWFWARNDPSVPSDIINGASSISPDGWGNPAALFPSTYCDFPSHFQQHNIIINLTLCACYESVGTKTNGLVLGGDWAGSAYGQSGCPSTCIDFVNNNPSAYTDAFFDFEYIWLYTP

SEQ ID NO: 72 - protein, 296 aa, Coprinopsis cinerea okayama
STYSLRKGIQGESFYDDFVWESIADPTHGRVNYVDQETSRWQNLTFATRDSFILRTDSTNIIPTDGPGRNSVRLRSKEKFKTHVSIFDVRHMPVGCGTWPAIWTVGENWPHGGEIDILEGVNDEAPNASTLHTGRGCTMPTENGPQTGHRMLNNCDASVNNNVGCPVDFPSHDSYGPGFNAAGGGWYVMERNDDFIKIWFWSRQDPNVPEDVKNPVWNVAPSNWGTPSAYFPGDSCNMSDFFAPHWIVINLTLCGDWAGTVYQYSNCPSTCVDHVNNNPWAFENAYFDIAQIRIYT

SEQ ID NO: 73 - protein, 323 aa, Glarea lozoyensis
SAASAAKIYTIDETYEGEGFFNKFNFFTGLDPTRGYVQYQSQADAASTKFGSKLVNTINGQNFMGVDHTNTYDPFGAGRPSVRIETKKTYNHGLFILDLAHMPSSTCGNWPAFWTYSDVNYPAQGEIDILENIHENTQSLNVLHTSAGFSVAGNKKGLQQSGDQTTYNCDDNAQSSDYGSQFTGQGCASTNINPGSYGSALNAVGGGVYAMEWTSDVIRVWSFPKVVIPLDIIAGKPDPSKWGLPTFTTAQGKGDIDSHFKDHKVVLDTTFCGNWAGQDFFWKQTSCYDPILYPTCSEYVGKNPSKYADTYWLINSLKVFQLN

SEQ ID NO: 74 - protein, 321 aa, Botryotinia fuckeliana
GEVSATNKYTLVDDYAGTNFFDMFDFYTGADPTTGFVSYTSKEVAENANSELGVALTKVENGQAYMGVDYVNVVTTGRPSVRIQSKESYTHGLIIADLAHMPASICGTWPAFWTVNTTNYPRYGEIDILENINENTVSLQTLHTEEGCYISGNQYSTQLKDNVTTYNCDDSASSSIFGAQEGNSACSGTNPDPNSYGTTFNSNGGGVYAMQWTSDVIRMWNFGPDAIPADITAGTPDPSTWDLPAFTTEGGVCNIDGLFANHHIIFDTTFCGVYAGKTKFWQETTCYDAEKYPTCDSYVGANPAAYKEAYWLINSVKVYQN

SEQ ID NO: 75 - protein, 300 aa, Aspergillus flavus
WAPLAAAAYTLQDDYGTDTTFFDKFSFFTGSDPTHGFVKYVDRGTAQNTGLIKADGTIYMGVDYTNAAPGGRQSVRISSNKVYNHGLFILDLAHMPGSICGAWPAYWLLGPNWPNNGEIDVIEGVNDQTNNQVALHTSDSCTINNSGFSGSLLTSNCYVNAPGQANNAGCGIKDNSAQSYGNGFNSAGGGVYATEWTGEAISVWFFPRSSIPGDISSGNPNPSGWGTPSARFAGACNIDSHFKDLQIIFDTTFCGDWAGGVWGSSSCASKGSCNDWVANNPAAFKDAFWRINSLKVYQGG

SEQ ID NO: 76 - protein, 300 aa, Aspergillus oryzae; identical to SEQ ID NO: 75

SEQ ID NO: 77 - protein, 302 aa, Talaromyces stipitatus
AGLSSAQTYSLVDNYPTGMDFFSKFSFFTDSDPTHGFVDYVSETTAKSAGLIYASGNGTYIGVDNTNVASSSGRQSVRLTSNAAYTKGLFVLDLAHMPGSVCGSWPAFWTVGSDWPNNGEIDIIEGVSQQSANAMTLHTSNGCSINDSGFTGHLATSNCYVNAAGQANNAGCSIDATTSATYGDAFNTNGGGIYAMEWTDNYIQVFEFSHATAPADINSNSPDPSNWGEPAARFQGNCDIDSHFSQHQIVFDITFCGDWAGNVWSSSTCSSYASTCNSYVQNNPSAFTESYWLINSLKVYQS

SEQ ID NO: 78 - protein, 305 aa, Arthroderma otae
LAELGSATYILEDDYQPNTWFDQFSFFSAKDPTHAYVNYLDQAAAQSQNLIGIQNNAVYLGVDHKNVATGEGRSSVRLETKKVYNHGLIIADINHMPGGECGTWPAFWTTSSAWPNEGELDIIEGVNQQKQNDYALHTAQGCSIPKQGEFTGTVVTPNCDVKAAGQAENQGCLVEDSKGSRGYGPDFNNATGGVFATEWTSKAISIWFFPREEIPKDVNSEHPDPSKWGKPSAYFGGECDVGNHVRNQRIIFNTAFCGGWADGMWPTDPVCSKKAPTCMEYVRENPSAFEEAYWSINYMKVYQEG

SEQ ID NO: 79 - protein, 306 aa, Trichopyton equinum
LAELGSATYILEDDYQPNTWFDQFRFFSAKDPTHAYVNYLDQAEARSQNLIGVRNNAVYLGVDHKNVATGEGRSSVRLETKKVYNHGLIVADINHMPGGECGTWPAFWTTSSAWPNEGELDIIEGVNQQKQNDYALHTAQGCSIPERGDFTGSVVTPNCDVKALGQAENQGCLVEDTRGSRGYGPDFNNATGGVFATEWTDQAISIWFFPREDIPNDVNSEHPDPSKWGKPSAFFGGGECPIGKHVRNQRIIFNTAFCGGWADGMWPGDPICSKKAPTCMEYVRENPSAFEDAYWSINYMKVYQQG

SEQ ID NO: 80 - protein, 306 aa, Trichopyton tonsurans; identical to SEQ ID NO: 79

SEQ ID NO: 81 - protein, 306 aa, Trychophyton verrucosum
LAELGSATYILEDDYQPNTWFDQFRFFSAKDPTHAYVNYLDQAEARSQNLIGVRNNAVYLGVDHKNVATGEGRSSVRLETKKVYNHGLIVADINHMPGGECGTWPAFWTTSSAWPNEGELDIIEGVNQQKQNDYALHTAQGCSIPERGDFTGSVVTPNCDVKALGQAENQGCLVEDTKGSRGYGPDFNNATGGVFATEWTEQAISIWFFPREDIPKDVNSEHPDPSKWGKPSAFFGGGECPIGKHVRNQRIIFNTAFCGGWADGMWPGDPICSKKAPTCMEYVRENPSAFEDAYWSINYMKVYQQG

SEQ ID NO: 82 - protein, 306 aa, Trichophyton rubrum
LAELGSATYILEDDYQPNTWFDQFRFFSAKDPTHAYVNYLDQAEARSQNLIGIRNNAVYLGVDHKNVATGEGRSSVRLETKKVYNHGLIVADINHMPGGECGTWPAFWTTSSVWPNEGELDIIEGVNQQKQNDYALHTAQGCSIPERGDFTGSVVTPNCDVKALGQAENQGCLVEDTKGSRGYGPDFNNATGGVFATEWTDQAISIWFFPRGDVPKDVNSEHPDPSKWGKPSAFFGGGECPIGKHVRNQRIIFNTAFCGGWADGMWPGDPICSKKAPTCMEYVRENPSAFEDAYWSINYMKVYQQG

SEQ ID NO: 83 - protein, 303 aa, Paecilomyces sp. J18
TSQLAAAYHLVDDYGRGNGFFDKFNFFTGDDPTHGYVDYVSRDVAAGAGLIGERDGRTYMGVDFTNPASGRGRRSVRLESKNTYEHGLIVIDLAHMPGSVCGTWPAFWTLGTGDWPYGGEIDIIEGVNDNTFNHMVLHTSDGCTIDNDGFTGNLKTSNCYVYAPGQDANAGCGIEATDPNSYGKGFNSIGGGIYATEITPNGISIWFFPRGSEPGDVLGDNPNPANWDTPAAKFAGGGCDWEGKFNAQRLIFDVTFCGDWAGNVWGIGGCASRAANCVDFVRDNPSAFAESYWLVNSLRVYAP

SEQ ID NO: 84 - protein, 306 aa, Arthroderma benhamiae
LAELGSATYILEDDYQPNTWFDQFRFFSAKDPTHAYVNYLDQAEARSQNLIGVRNNAVYLGVDHKNVATGEGRSSVRLETKKVYNHGLIVADINHMPGGECGTWPAFWTTSSAWPMEGELDIIEGVNQQKQNDYALHTAQGCSIPERGDFTGSVVTPNCDVKALGQAENQGCLVEDTKGSRGYGPDFNNATGGVFATEWTDQAISIWFFPREDIPKDVNSEHPDPSKWGKPSAFFGGGECPIGKHVRNQRIIFNTAFCGGWADGMWPGDPICSKKAPTCMEYVRENPSAFEDAYWSINYMKVYQQG

SEQ ID NO: 85 - protein, 302 aa, Neosartorya fischeri
SATGMAAYVLEDDYGTSTSFFDKFSFFTDPDPTGGFVSYVDRNTAQKAGLISANGAVYMGVDHTNVAGSSGRQSVRLTSTKSYTHGLVILDLAHMPGGICGTWPAFWLLGPDWPSHGEIDIIEGVNTQSTNQMTLHSTDGCSIANGGFTGTLLTSNCYDYAPGQETNAGCSIAATSSLTYGTGFNNAGGGIYATEWTSAGISIWFFPRGSTPLDIRAGTPDPTNWGTPLAKFAPGSCDFDAHFSEMQLVFDTTFCGGWAGAVWGSGSCASVESSCQDFVANNPSVFQEAYWLINSLKVYQDA

SEQ ID NO: 86 - protein, 306 aa, Aspergillus fumigatus
LEARQSQTYQLAESWQGESFINDWNFFDGADPTNGYVTYVNQSFAKQSGLVKVTESGSFYMGVDYESTLNPNGAGRESVRIESKNYYTEGLYVIDIEHMPGSICGTWPAFWSVGKNWPNDGEIDIIEGVNLQKANKIVLHTSGSCDVSGSNDMTGTLSSSECGEASGTVGCVVKGTNGSSGDPFNESGGGVYAMEWTDTFIKIWFFPRSQIPASLASGNPDTSSFGTPMAHLQGSCDFAERFKAQKLIIDTTFCGDWAGNVFAESTCPMSDPSSPMQSCVNYVAQNPAAFKEAYWEINSIKIYQYG

SEQ ID NO: 87 - protein, 306 aa, Arthroderma gypseum
LAELGSATYILEDDYQPSTWFDQFRFFSAKDPTHAYVDYLDQAEARSQNLIGIKNNAVFLGVDHTNIATGEGRRSVRLETKKVYNHALIVADINHMPGGECGTWPAFWTTSSAWPREGELDIIEGVNQQKQNDYALHTAQGCSIPGQGDFTGTVVTPNCDVKAAGQAENQGCLVEDSKGSRGYGPDFNNATGGVFATEWTDQAISIWFFPREEIPKDINSEHPDPSKWGKPSAFFGGSQCPIGNHVRNQRIIFNTAFCGGWADGMWPGDPVCSKKAPTCMEYVRENPSAFEDAYWSINYMKVYQQG

SEQ ID NO: 88 - protein, 303 aa, Aspergillus nidulans
IAHAASNYKLKESWEGEKILNHFHFFDNADPTNGFVTYVNQSYAESAGLVKTTDSGSLYLGVDYENVLTVDGPGRESVRIESNEYYDQGLYVVDIQHMPGSICGTWPAFWTVGPDWPTDGEIDIIEGVNKHDANKIVLHTSDTCDVGGGYKMTGDMTSSECGEASGTIGCVVQGKQGSSGDPFNEQGGGVYAMEWQEKYLKIWYFPRSSIPESLTAGTPDVSSFGTPMAHLQGSCNFKERFTHQKLILDTTFCGDWAGGVFGDSGCPVSDPSDPMLSCKNYVAENPAVYKNAYWELNSIKIYQ

SEQ ID NO: 89 - protein, 297 aa, Penicillium funiculosum
AVAYTLHWDVNSSNFLDYFVFDTEADPTAGFVKYVDQSTASNDGLYSTSNNQIYLGVDKTTVLDSSSTGRNSVRVYSQNTFSSGILITDFAHLPVSVCGIWPAYWTINNQANPYGEIDIMEAYDDVAGAYVSLHTSNTCTLSNRNFTGTDTRTDCTLSSGGGCGVQSTSSQFGAGFNAAGGGVWVLSLENSLQLWVFPRNQIPADITNGSPNPSSWGTPLFEFDSNNGCDVSSNFIDQTVIFNLDFCGQNGAGGQEWSDWTDCATTTGQSTCNAYVAANPSAYSETYFSINSIKLYQ

SEQ ID NO: 90 - protein, 298 aa, Penicillium purporogenum
VAAYKLQWDVTSSNFLDYFVFDTETDPSNGFVTYVDESRASSGGLYSTSNGQIFLGVDNTTVLDSSATGRNSVRVYSQNTFSSGILITDFKHLPVAVCGIWPAYWTINNLADPYGEIDIIEAYDDVSNAYTSLHTSSNCTVSDTDFTGTDVRTDCTLSTSASGCGVESTASQFGAGFNSAGGGVWVLSLSDSLQIWVFTRDNIPADITDGSPNPSGWGTPLFEFDSTSDCGVSSNFLNQTVIFNIDFCGEEDAGGKEWATWTDCLATTGVSTCNAYVAANPATYSETNFVINSIKLYQ

SEQ ID NO: 91 - protein, 301 aa, Schizophyllum commune
DVNANGSTFLWLLEDDFSGDDFFNNFGFFTGEDPTHYVDANTAFGSGLSYVQDDGIVVMKGDNTSWLGDGEYRKSVRISSYKQYNTGLFILDLNKAPWGCAVWPAFWTLGSGTWPQTGEIDIIEGVHDNEHNQVAWHTASGCYLDPTASFTGTVVVQNNTNCDGSVNSNAGCAIAEWSRASYGPYFDAQGGGVFAMKWDENGIAVYSFYRAAIPDDINAGSPNPSGWGKPVAFLSPDSCDPIKYFTNHSIIFDITFCGDWAGNSYATSGCPGECSDRLKDPANFVNASWSINSLKVYSKQP

SEQ ID NO: 92 - protein, 336 aa, Piriformospora indica
FSQTVCGYNLVQTYSGSSFFDSWTFADGWDHWTNGDIIYLPQAQAGNLTRLTENGNVQIRMDDFTGLGDNMKRNSIRIEGKQIFNSTNAVVVFDVVHVPTGCSVSNTGKGGSLWSKSAIFVNDWPKGGEVDIMEAVNKMTTNQMALHTDISCTMSTSNPNQSGNAGNADCQVTFGPDGKAINPQGCTVLDTQPQSYSTLAESGGGVWVAEYGAQALNIWFFPRANVPDALKGTPETLDTAALGKPVANYPSSSTCDISKALYPQHLVIDVTACGDCKFGGSMNRGFSFFLGAKGVLESTGCTPSLPNDCYNSYVHLPDNYHDAYCEWIHHSFCVSN

SEQ ID NO: 93 - protein, 313 aa, Aspergillus fumigatus
ALEARQSQTYQLAESWQGESFINDWNFFDGADPTNGYVTYVNQSFAKQSGLVKVTESGSFYMGVDYESTLNPNGAGRESVRIESKNYYTEGLYVIDIEHMPGSICGTWPAFWSVGKNWPNDGEIDIIEGVNLQKANKIVLHTSGSCDVSGSNDMTGTLSSSECGEASGTVGCVVKGTNGSSGDPFNESGGGVYAMEWTDTFIKIWFFPRSQIPASLASGNPDTSSFGTPMAHLQGSCDFAERFKAQKLIIDTTFCGDWAGNVFAESTCPMSDPSSPMQSCVNYVAQNPAAFKEAYWEINSIKIYQYGVSAASS

SEQ ID NO: 94 - protein, 337 aa, Myceliophthora thermophila
AVVGVKATRNNGGGNSSYPDYFKLNYTLIDTYSGTTFFDKFNYFHTWDPAGGFVHYVDPDYAGTYNLTWATSSTALIRVDTTVGPGSEPDASTGRFSVRLESKTQYGPGLFLFDVKHTPYGCATWPALWLTDPNNWPDNGEIDVMEAVNQAAAGTLVALHTTAGCTMADVRRDMAGAAEQADCHNATNSNAGCAVRGAPATYGPEFNEAGGGIVALEWRAEGIRVWVFPRGGSGSGSGGGDERGLAALPAAEAKETPDPSAWGPPLADFPATSCDVGSHFRNQSIIINIDLCGYLTEAVWEESGCSRLKCIDFVANNPSAFTNAYWEFGAFQVYKAQ

SEQ ID NO: 95 - protein, 333 aa, Thielavia terrestris
AVVGVRATRNNNPDGTSSSYPDYTQLNYTLIDTYSGTSFFDKFEYFSRSDPTQGFVHYVDPGYAATYNLTYATQSTAIIRVDTTVGPGSNPDASTGRFSVRLESKAQYGPGLFLFDVKHTPYGCGTWPALWLTDPSNWPENGEIDLMEAVNQASAGGLTALHTTAGCTMADVRREMSGAAGQDDCHNATNSNTGCTVTGGAAMYGPAFNAAGGGVVALEWRAEGIRVWVLGRDGGGGGKGVITALPAAEQLAGPDTGTWGPPLADFPSTSCDVTSHFRNQSIIVNIDLCGELPNAVWASSGCPSNCTDYVANNPLAFTNAYWEFGAFQVYKAA

SEQ ID NO: 96 - protein, 314 aa, Botryotinia fuckeliana
AVEGSKKNAYPDYSQLNYTLKDTYSGTDFFDNFDYFNTYDPSAGFVHYVDSEVAAQYNLTYASSSSAVVRVDTSVTADSNPNASTGRFSVRIESKTQYTDGLFIFDIVHTPIGCATWPALWLSDPNNWPTNGEIDIMEAVNVVSSTKNQMTLHTTSGCSMDVKRKETGKSIQSSCLNSTNSNAGCGVYDSAGTFGADFNSNGGGVMAMELRTAGIRMWQFGRDAIPTDISSGSPDPSTWSEATADFPSTNCNIGNHFRNQSIIVNIDLCGSWAGTESVYDVDCPGTCTDYVANNATAFTDAYWQFNNFTVYQAS

SEQ ID NO: 97 - protein, 374 aa, Myceliophthora thermophila
STARAGSYTLVDTFDASNFFDEFDFFTEPDPTHGFVQYVDGDTANREGLAGFASGGVYLGVDYSSTTTTTTTGRASVRLTSRKAYTRGLFVADIAHMPAGAAGSSSCGLWPAFWMFGPDWPNSGEIDVVEGVNSQTSNSVSLHTGAGCTVSNPGASPGTKLVSADCQGGEGCTQDTSAPDNYGAGFNAAGGGIYAVEWTDAAIKVWFLPRDSPIASQLSSSFSSSSSSSFPSGGNNNNTNIKDNKSLDPSAFGTPLAVFAGGAECPIGDHFANHHLVFDTTFCGDWAGRVWAADGACAALADTCEDYVAAHPEAFSEAYWLLGSIRVYQLEADAGAGTGTGGASAGGGDDGQGEGGGQGQRRGLRPKMARRWQA

SEQ ID NO: 98 - protein, 373 aa, Botyootinia fuckeliana
EVSATNKYTLVDDYAGTNFFDMFDFYTGADPTTGFVSYTSKEVAENANSELGVALTKVENGQAYMGVDYVNVVTTGRPSVRIQSKESYTHGLIIADLAHMPASICGTWPAFWTVNTTNYPRYGEIDILENINENTVSLQTLHTEEGCYISGNQYSTQLKDNVTTYNCDDSASSSIFGAQEGNSACSGTNPDPNSYGTTFNSNGGGVYAMQWTSDVIRMWNFGPDAIPADITAGTPDPSTWDLPAFTTEGGVCNIDGLFANHHIIFDTTFCGVYAGKTKFWQETTCYDAEKYPTCDSYVGANPAAYKEAYWLINSVKVYQNDTLAATSAAAASSTSAAIVATSTKISTTASPVMSSTARAESLLGVATSEAASI

SEQ ID NO: 99 - protein, 355 aa, Thielavia terrestris
CSVRAASQYGLVDVYDASNFFTEFDFFTQPDPTNGFVKYVDAATANRDGLAGFTEGGVYLGVDYTNTTTTGRRSVRLTSKKAYTKGLFIADIAHMPAGATGSGSCGLWPAFWMFGPNWPNSGEVDVIEGVNSQTSNSVSLHTGAGCTISNTGTISTTKLLAANCQGNEGCTQQTTSSDNYGTGFNAAGGGVYAVEWTSAAIKVWFFPRGSPVATQLAAGADATSSSSSSSSSSSSSAASSPDPSTFGPPLAAFVGGPTCSIDQHFANHNLVFDTTFCGDWAGRVWADDETCSALASTCEDFVGQNPGAFTQAYWLVNGLRVYQAGGEAQGRQGRQQLRKVGFRRGGPAEGRRWRA

SEQ ID NO: 100 - protein, 297 aa, Rhizopus oryzae
SVHAWTLKQTFQGSSFFDGFTFFTDSDPTHGFVKYVDQATAKSSGLIYNQGSKVIMRADNTTVSGRKSVRITSKSSYSNSALPNVLLDLEHMPVGCGTWPAFWMVGPNWPNSGEIDIIENVNEATVNQVTLHTKNGCTMAGVPRTQTGKSLTDNCYVNAAGQANNAGCGVQATNTNTYGKGLNNIKGGVYATRMTASQGVQVWFFPRNNIPSDISSGSPNPPSWPAPIASFPFQSGSCSISYFSQLQIVFDLTFCGDWAGSVYSSSGCPSSCNDYVANNPRSFTNSYWSINYVKVYQ

SEQ ID NO: 101 - protein, 328 aa, Aspergillus nidulans
ELASAAYVLQDDYSPDVFFDKFTFFTDADPTHGHVDYVDRGTAQSAGLISSGSSVYMGVDHTNIASSGRQSVRLSSTQTYHHGLFIIDLSHMPTGCGTWPAFWILGPDWPNGGEIDVIENVNVATNNHMTLHTSDGCTIDSSGFTGTLLTSNCFVNAPGQANNAGCGIQSPDSNSYGAGFNSNSGGVYATEWTSDHISIWFFPRSSIPSDITAGNPDPSTWGTPAARFAGNCDIESHFTDMQIIFDITFCGDWAGNVWESSTCASLGSCTDYVSNNPEAFADAYWDINSLRVYQDSAAAKRDEIEGREKTSAKGFPRKSMRARRDAGL

SEQ ID NO: 102 - protein, 352 aa, Penicillium chrysogenum
QVSSAAYTLRDDYGASDSFFDKFNFFTDTDPTNGFVSYVDRSTASRNGLINTGNGVYIGVDHANIATRPGRQSVRLESTATYKHGLVILDLAHMPSSTCGTWPAFWMLGPNWPNNGEIDIIEGVNEQTQNQVALHTSNGCTINNSGFTGNLETPNCYVQAPGQSANSGCVIQDGSTQSYGTGFNNVGGGVYATEWTGSAISVWFFPSYAVPADISSGNPNPAGWGTPSARFAGGCNIDSKFNDLQIVFDITFCGDWAGSVWGSSSCASRAWSCVDYVQNNPTAFQESYWRVRSLKVYQDAASSKGGAEGEDVGAGDEVGVNWDPSTWYGLSARASMKHRREHYRHKRGHGHA